(19) United States
(12) Patent Application Publication
(10) Pub. No.: US 2025/0270374 A1
(43) Pub. Date: Aug. 28, 2025

(54) BIOACTIVE GRANULAR HYDROGEL SCAFFOLDS AND USE THEREOF

(71) Applicant: The Penn State Research Foundation, University Park, PA (US)

(72) Inventors: Amir Sheikhi, University Park, PA (US); Arian Jaberi, University Park, PA (US); Mohammad Zaman Ataie, University Park, PA (US); Alexander Kedzierski, University Park, PA (US); Sina Kheirabadi, University Park, PA (US)

(21) Appl. No.: 19/208,226

(22) Filed: May 14, 2025

Related U.S. Application Data
(63) Continuation-in-part of application No. 18/848,678, filed on Sep. 19, 2024.
(60) Provisional application No. 63/324,774, filed on Mar. 29, 2022.
(60) Provisional application No. 63/367,521, filed on Jul. 1, 2022.
(60) Provisional application No. 63/424,286, filed on Nov. 10, 2022.
(60) Provisional application No. 63/647,774, filed on May 15, 2024.
(60) Provisional application No. 63/647,744, filed on May 15, 2024.

(51) Int. Cl. C08J 3/075; A61L 27/18; A61L 27/22; A61L 27/52; A61L 27/56; C08J 3/24; C08L 71/02; C08L 89/06

(52) U.S. Cl. CPC C08J 3/075; A61L 27/18; A61L 27/222; A61L 27/52; A61L 27/56; C08J 3/246; C08L 71/02; C08L 89/06; A61L 2400/06

(57) ABSTRACT

Embodiments relate to porous hydrogel microparticles, porous granular hydrogel scaffolds, and methods of making and using thereof. A method of making porous hydrogel microparticles includes crosslinking first polymers and second polymers to form composite microgels, adding the composite microgels to a liquid solution at a first temperature to form a composite microgel suspension, reducing the temperature of the composite microgel suspension to a second temperature below a phase separation temperature such that the second polymers separate from the first polymers, and filtering the composite microgel suspension from the liquid solution such that the second polymers diffuse out of the composite microgel suspension, resulting in the porous microgels.

### CROSS-REFERENCE TO RELATED APPLICATIONS

This patent application is a continuation-in-part application of U.S. application Ser. No. 18/848,678, filed on Sep. 19, 2024, which is the U.S. National Stage Application of International Patent Application No. PCT/US2023/016658, filed on Mar. 29, 2023, which is related to and claims the benefit of priority of U.S. Provisional Application No. 63/324,774, filed on Mar. 29, 2022, and is further related to and clams the benefit of priority of U.S. Provisional Application No. 63/367,521, filed on Jul. 1, 2022, and is further related to and claims the benefit of priority of U.S. Provisional Application No. 63/424,286, filed on Nov. 10, 2022, the entire contents of which are incorporated by reference. This patent application is further related to and claims the benefit of priority of U.S. Provisional Application No. 63/647,744, filed on May 15, 2024, and is further related to and clams the benefit of priority of U.S. Provisional Application No. 63/647,774, filed on May 15, 2024, the entire contents of which are incorporated by reference.

### STATEMENT REGARDING FEDERALLY SPONSORED RESEARCH DEVELOPMENT

This invention was made with government support under Grant Nos. NS121150 and HL167939 awarded by the National Institutes of Health. The Government has certain rights in the invention.

### FIELD OF THE INVENTION

Embodiments relate to hydrogel microparticles, bioactive granular hydrogel scaffolds, and methods of making and using thereof.

### BACKGROUND OF THE INVENTION

Granular hydrogel scaffolds (GHS) have enabled rapid cell infiltration and downregulated inflammatory responses during tissue regeneration. Various polymers have been used as the building blocks of GHS, including hyaluronic acid (HA), polyethylene glycol (PEG), and gelatin methacryloyl (GelMA), via different crosslinking and assembly approaches. GelMA is methacryloyl-modified gelatin that can undergo physical (e.g., thermal) and chemical (e.g., free radical polymerization) crosslinking, providing a biocompatible network decorated with the cell-adhesive RGD motifs. GelMA GHS have been used for tissue engineering and 3D bioprinting.

### BRIEF SUMMARY OF THE INVENTION

Embodiments relate to exemplary formulations and methods of converting protein/peptide-based materials and/or any other synthetic or semi-natural material, including carbohydrates and their derivatives, to granular hydrogel scaffolds. In some embodiments, scaffold formation does not require light exposure. A polymer can first be converted to stable microgels (micro-scale hydrogel particles) via chemical crosslinking (e.g., step 1), followed by microgel-microgel assembly using orthogonal, non-light-mediated crosslinking (e.g., step 2). Exemplary step 1 may involve a range of chemical crosslinking methods, such as free-radical polymerization of vinyl groups and any other technique. Exemplary step 2 may be based on the crosslinking of other functional groups that were not used in step 1, such as amines, using enzymes, dynamic covalent bond formation, or any other method.

Embodiments further relate to polymeric granular hydrogel scaffolds that may be formed inside of tissues that do not have access to light. Embodiments may provide additional, new opportunities for noninvasive or minimally invasive tissue regeneration using granular hydrogel scaffolds without requiring open surgery.

Embodiments further relate to microgels that may be decorated and/or encapsulated with other biological factors or nano-structure materials to further promote the biological function of granular hydrogels.

It is one object of the present disclosure to provide methods and formulations of converting polymers, such as proteins and/or peptides, to hydrogel microparticles that can form granular hydrogel scaffolds after injection in tissues.

It is a further object of the present disclosure to provide methods and formulations of crosslinking hydrogel microparticles in a way that they remain stable at the physiological temperature and undergo assembly after enzymatic activation to form granular hydrogel scaffolds.

It is a further object of the present disclosure to provide methods of crosslinking hydrogel microparticles such that they remain stable at the physiological temperature and undergo assembly after mixing with another polymer, such as aldehyde-modified hyaluronic acid, to form granular hydrogel scaffolds.

It is a further object of the present disclosure to provide methods and formulations of decorating hydrogel microparticles with biologics (e.g., growth factors, drugs, cells), followed by the hydrogel microparticle assembly to form granular hydrogel scaffolds with enhanced bioactivity (e.g., bioactive granular hydrogel scaffolds).

It is a further object of the present disclosure to provide methods and formulations of encapsulating biologics (e.g., growth factors, drugs, cells) in hydrogel microparticles, followed by hydrogel microparticle assembly to form bioactive granular hydrogel scaffolds.

It is a further object of the present disclosure to provide methods and formulations to form in situ granular hydrogel scaffold composites/nanocomposites that mimic the physicochemical and/or biological characteristics of native tissues, such as brain, skin, muscle, etc.

Embodiments further relate to porous microgels, porous hydrogel scaffolds, and methods of making and using thereof. It is one object of the present disclosure to provide porous microgels such that voids are incorporated into the scaffold structures. As the microgels serve as the building blocks of scaffolds, porous microgels may impart (or increase) porosity to the scaffold itself and may enhance the void fraction of the scaffold compared with scaffolds formed from nonporous microgels.

Embodiments further relate to hybrid (e.g., cell-microgel) aggregates. In particular, cells may serve as assembly engines and migrate/adhere to the microgels such that a self-assembly process is initiated and aggregates are formed. In particular, microgels may be significantly larger than the cells such that porous aggregates are formed. Porous aggregates may enhance molecular diffusion and improve cell viability.

In an exemplary embodiment, a method of forming porous microgels comprises crosslinking first polymers and second polymers to form composite microgels; adding the composite microgels to a liquid solution at a first temperature to form a composite microgel suspension; reducing the temperature of the composite microgel suspension to a second temperature below a phase separation temperature such that the second polymers fully or partially separate from the first polymers; and filtering the composite microgel suspension from the liquid solution such that the second polymers diffuse out of the composite microgel suspension, resulting in the porous microgels.

In some embodiments, the first polymers and the second polymers are two different polymers selected from the group consisting of hyaluronic acid, polyethylene glycol, and gelatin methacryloyl.

In some embodiments, the first polymers are gelatin methacryloyl and the second polymers are polyethylene glycol.

In an exemplary embodiment, a porous microgel is formed according to the method described above.

In some embodiments, voids of the porous microgel are between 5 and 40 μm.

In an exemplary embodiment, a method of forming a porous granular hydrogel scaffold comprises providing porous microgels according to the method described above; and crosslinking the porous microgels to form the porous granular hydrogel scaffold.

In some embodiments, crosslinking the porous microgels comprises physical crosslinking.

In some embodiments, crosslinking the porous microgels comprises chemical crosslinking.

In some embodiments, crosslinking the porous microgels comprises non-light-mediated crosslinking.

In an exemplary embodiment, a porous granular hydrogel scaffold is formed from the method described above.

In some embodiments, the porous granular hydrogel scaffold has a void fraction between 15% and 60%.

In an exemplary embodiment, a method of forming a porous granular hydrogel scaffold comprises providing porous microgels according to the method described above; combining the porous microgels with adherent cells to form hybrid microgel aggregates; and crosslinking the hybrid microgel aggregates to form the porous granular hydrogel scaffold.

In some embodiments, crosslinking the porous microgels comprise physical crosslinking.

In some embodiments, crosslinking the porous microgels comprise chemical crosslinking.

In some embodiments, crosslinking the porous microgels comprises non-light-mediated crosslinking.

In some embodiments, the hybrid microgel aggregates have void fractions between 3 and 30%.

In an exemplary embodiment, a method for regenerating tissue comprises providing porous microgels according to the method described above; injecting the porous microgels at an injection site within the tissue; and crosslinking the porous microgels to form the porous granular hydrogel scaffold.

In some embodiments, crosslinking the porous microgels comprise physical crosslinking and/or chemical crosslinking.

In some embodiments, crosslinking the porous microgels comprises a non-light-mediated crosslinking, and wherein the injection site does not have access to light.

In some embodiments, the tissue is selected from the group consisting of nervous tissue, endothelial tissue, epithelial tissue, muscle tissue, and connective tissue.

Further features, aspects, objects, advantages, and possible applications of the present invention will become apparent from a study of the exemplary embodiments and examples described below, in combination with the Figures, and the appended claims.

### BRIEF DESCRIPTION OF THE FIGURES

The above and other objects, aspects, features, advantages, and possible applications of the present invention will be more apparent from the following more particular description thereof, presented in conjunction with the following drawings. It should be understood that like reference numbers used in the drawings may identify like components.

FIG. 1 shows an exemplary method for forming an embodiment of the granular hydrogel scaffolds.

FIG. 2A shows a schematic of individual gelatin methacryloyl hydrogel microparticle photocrosslinking and the mechanism of hydrogel microparticle assembly via activated factor XIII-mediated glutamyl-lysine bond formation.

FIG. 2B shows gelatin methacryloyl droplets and hydrogel microparticles (photocrosslinked droplets), prepared in three sizes: small, medium, or large (scale bar is 200 μm).

FIG. 2C shows the average diameter of gelatin methacryloyl droplets and hydrogel microparticles.

FIG. 2D shows the orthographic view and pore identification of gelatin methacryloyl granular hydrogel scaffolds, fabricated using small, medium, or large hydrogel microparticles (scale bar is 200 μm).

FIG. 2E shows gelatin methacryloyl granular hydrogel scaffolds void fraction.

FIG. 2F shows equivalent median pore diameter of gelatin methacryloyl granular hydrogel scaffolds.

FIG. 2G shows injectability of packed gelatin methacryloyl hydrogel microparticles (scale bar is 200 μm).

FIG. 3A shows optical images of small, medium, or large photocrosslinked gelatin methacryloyl hydrogel microparticles with gelatin methacryloyl concentration of 1.5% (w/v) and 60 s of UV exposure at the intensity of 15 mW cm⁻² incubated at 37° C. for up to 24 h (scale bar is 200 μm).

FIG. 3B shows diameters of hydrogel microparticles.

FIG. 4A shows frequency sweep tests to measure the storage modulus of bulk gelatin methacryloyl hydrogel (concentration=1, 1.5, 2, or 3% w/v in 4-(2-hydroxyethyl)-1-piperazineethanesulfonic acid, resembling hydrogel microparticles), photocrosslinked via UV light exposure for 30 s.

FIG. 4B shows frequency sweep tests to measure the storage modulus of bulk gelatin methacryloyl hydrogel (concentration=1, 1.5, 2, or 3% w/v in 4-(2-hydroxyethyl)-1-piperazineethanesulfonic acid, resembling hydrogel microparticles), photocrosslinked via UV light exposure for 60 s.

FIG. 4C shows the storage modulus of bulk gelatin methacryloyl scaffolds at a frequency of 1 rad s⁻¹ and oscillatory strain of 0.1%. The data represents mean±standard deviation for at least 3 samples.

FIG. 4D shows compressive modulus of gelatin methacryloyl granular hydrogel scaffolds assembled via reacting medium gelatin methacryloyl hydrogel microparticles with activated factor XIII (concentrations of 0, 2.5, 5, or 10 U mL⁻¹) for 1.5 h. The data represents mean±standard deviation for at least 3 samples.

FIG. 4E shows the effect of activated factor XIII (5 U mL⁻¹) reaction time at 37° C. on the compressive modulus of gelatin methacryloyl granular hydrogel scaffolds. The data represents mean±standard deviation for at least 3 samples.

FIGS. 5A-C show in situ fabrication of granular hydrogel scaffolds from thermoresistive GelMA microgels (scale bar is 200 μm).

FIGS. 6A-D show material and mechanical characterization of granular hydrogel scaffolds made from thermoresistive GelMA microgels.

FIGS. 7A-C show biocompatibility assessment of granular hydrogel scaffolds made from thermoresistive GelMA microgels.

FIG. 8 shows an exemplary method for regenerating tissue via the granular hydrogel scaffold.

FIG. 9 is a schematic illustration showing droplets of a homogenous binary solution of GelMA (7% w/v) and PEG (1.5, 2, or 2.5% w/v) polymers at Tᵢ, fabricated using a high-throughput step emulsification microfluidic device. The scale bar is 200 μm.

FIG. 10 is a schematic illustration showing droplets converted to microgels by reducing temperature to T_{f}, initiating the thermally induced phase separation of GelMA and PEG. The phase separation is driven by the miscibility reduction of GelMA and PEG in an aqueous solution, yielding two distinct phases until halted by GelMA polymer physical gel formation.

FIG. 11 is a schematic illustration showing phase separation temperature, T_{PS}, of GelMA-PEG mixtures at varying PEG concentrations (1.5, 2, 2.5% w/v) and a fixed GelMA concentration (7% w/v).

FIG. 12 is a schematic illustration demonstrating that the removal of oil and surfactant enables PEG chains to diffuse out of the physically crosslinked GelMA microgels (shown with arrows), leading to the formation of interconnected pores and rendering the microgels porous.

FIG. 13 is a schematic illustration showing GHS with a hierarchical porous structure may be fabricated via the free radical photopolymerization of jammed porous microgels, forming intra-and inter-microgel covalent bonds.

FIG. 14 shows bright-field microscopy images of physically-crosslinked microgels with varying porosity, fabricated via the phase separation of GelMA (7% w/v) and PEG polymer mixtures at varying PEG concentrations (1.5, 2, or 2.5% w/v) and T_{f}. Microgel size (average diameter˜187±10 μm, number of analyzed microgels n>5000) is independent from microgel porosity, and the particles have a narrow size distribution. The dashed and solid lines show the median and quartiles in the datasets, respectively. The scale bar is 200 μm.

FIG. 15 shows fluorescence microscopy images of individually photocrosslinked microgels that initially contained PEG (2% w/v), incubated with FITC-dextran (average molecular weight=2 MDa). Microgels are shown in cross-sectional 2D slices and 3D images that are constructed from Z-stacks. The scale bar is 50 μm.

FIG. 16 is a graph showing average median pore size of porous microgels (initially contained 2% w/v PEG), calculated using a MATLAB script (n>15).

FIG. 17 is a graph showing void fraction of porous microgels (initially contained 2% w/v PEG), calculated using a MATLAB script (n>15).

FIG. 18 shows fluorescence images of GHS, made up of microgels with varying pore sizes, acquired using a fluorescent molecule (FITC-dextran, average molecular weight=2 MDa). The scale bar is 100 μm.

FIG. 19 is a graph showing void fraction (n=5) of GHS, fabricated using the porous or nonporous microgels. The analysis was conducted using a MATLAB code.

FIG. 20 is a graph showing pore size (n=5) of GHS, fabricated using the porous or nonporous microgels. The analysis was conducted using a MATLAB code.

FIG. 21 is a graph showing compressive stress-strain curves of scaffolds.

FIG. 22 is a graph showing compressive modulus of GHS with hierarchical (GHS-S and GHS-L) and non-hierarchical (GHS-N) porous structures (n=5).

FIG. 23 is a graph showing dynamic moduli of GHS, fabricated using porous or nonporous microgels versus angular frequency.

FIG. 24 is a graph showing G′ acquired at angular frequency ˜1 rad s⁻¹ and strain ˜0.1% (n=5).

FIG. 25 is a graph showing G″, acquired at angular frequency ˜1 rad s⁻¹ and strain ˜0.1% (n=5).

FIG. 26 is a schematic illustration showing individual microgel photocrosslinking, followed by mixing with NIH/3T3 murine fibroblast cells and culturing.

FIG. 27 shows NIH/3T3 murine fibroblast cells, stained with calcein AM, imaged via fluorescence microscopy. The 2D slices and 3D-rendered images of microgels, showing cell adhesion to the non-porous or porous microgel exterior as well as cell infiltration into the porous microgels. The scale bar is 100 μm.

FIG. 28 is a graph showing cell volume, calculated by the summation of total cell area in each 2D image multiplied by the Z-step size in each microgels, assessed using a MATLAB code (number of analyzed microgels per group n>15). Dashed and solid lines indicate the median and quartiles, respectively.

FIG. 29 shows a live/dead assay, conducted at varying culture periods. The scale bar is 500 μm.

FIG. 30 shows graphs demonstrating cell viability in cell-laden GHS. A high cell viability (>95%) is observed within 7 days of culture for all the study groups (n=3) (top), and metabolic activity of NIH/3T3 murine fibroblast cells in the scaffolds, measured using the PrestoBlue assay (bottom). In all study groups, metabolic activity significantly increased on days 4 and 7 compared with day 1 (n=5).

FIG. 31 is a schematic illustration of the subcutaneous implantation mouse model to evaluate endogenous cell infiltration into acellular scaffolds. Dashed lines indicate varying scaffold depths.

FIG. 32 shows representative images of stained cell nuclei, showing the distribution of infiltrated cells (labeled with DAPI) into the scaffolds, acquired 2 weeks after scaffold implantation. Dashed lines show the tissue-scaffold interface. The scale bar is 200 μm.

FIG. 33 is a graph showing cell density in GHS, measured as the ratio of DAPI-stained nucleus area over the total ROI area. A higher cell density is observed in GHS-L and GHS-S compared with GHS-N.

FIG. 34 shows graphs demonstrating cell infiltration profile at varying depths of (left) GHS-N, (center) GHS-S, and (right) GHS-L, normalized with the total ROI area (length˜400 μm and height˜200 μm). The highest cell density across all study group is yielded in the top 200 μm layer adjacent to the tissue, and no significant changes are observed thereafter.

FIG. 35 shows images of infiltrated cells, stained with α-SMA, CD31, or CD68. Myofibroblasts, endothelial cells, and macrophages are confirmed across all study groups. White dashed lines indicate the tissue-scaffold interface. The scale bar is 200 μm.

FIG. 36 shows graphs demonstrating coverage area of infiltrated cells, stained with (top) α-SMA, (middle) CD31, or (bottom) CD68 in varying study groups. A significantly higher CD31fluorescence area is observed in GHS-S and GHS-L compared with GHS-N. Additionally, α-SMA fluorescence area in GHS-L is significantly higher than that in GHS-N.

FIG. 37 is a schematic illustration showing GelMA droplets photochemically crosslinked to form stable microgels.

FIG. 38 shows GelMA droplets and their corresponding microgels formed via photocrosslinking in three distinct sizes: small (left), medium (center), and large (right). Fluorescent images show the size distribution of droplets and resulting microgels. Histograms presenting the relative frequency (%) of each droplet and microgel size.

FIG. 39 is a schematic illustration demonstrating that interactions between cells and ECM or GelMA influence the formation of cell aggregates, including cell spheroid and BHS. The adhesion is regulated by homophilic binding between cadherin moieties, and integrin-mediated cell-biomaterial and cell-ECM interactions.

FIG. 40 is a schematic illustration showing NIH/3T3 murine fibroblast cells and GelMA microgels are mixed to initiate cell-mediated microgel assembly (step 1). Initially, cells adhere to the microgels because of the adhesive moieties on GelMA (step 2). CS are formed by cell-cell aggregation, and BHS are yielded by cell-microgel assembly (step 3). Pseudo-colored optical microscopy images of cells and GelMA microgels, showing the formation of two types of aggregates.

FIG. 41 shows optical microscopy images NIH/3T3 murine fibroblast cells cultured with varying microgel sizes on a planar, non-adhesive substrate for 72 h. The scale bar is 300 μm.

FIG. 42 shows graphs demonstrating representative x-y trajectories and relative angles for the selected microgel pairs. The black solid lines denote the trajectories of the center of mass, and the line segments denote the relative angle θ between two microgels.

FIG. 43 is a graph showing remaining single cell percentage for small, medium, and large microgels. Inset: the same data plotted in logarithmic scale. The exponential fits are shown by the solid lines.

FIG. 44 is a graph showing characteristics decay time (τ) for the formation kinetics of BHS with varying sizes of microgels.

FIG. 45 is a graph showing the equivalent radii R_{eq} of BHS, formed using the small, medium, or large microgels after 72 h of culture.

FIG. 46 shows schematic illustration of BHS or cell spheroids formed by mixing cells with varying sizes of microgels in a U-bottom microwell plate (left). Confocal microscopy images of BHS or cell spheroid after 5 days, with actin filaments, nuclei, and microgels. (left-center). Nucleus density heatmap correlated with confocal microscopy images. (right-center) SEM images of aggregates, showing that cell spheroid and BHS-S have compact spherical shapes, while BHS-M and BHS-L were porous because of the larger building blocks. (right)

FIG. 47 is a graph showing metabolic activity of cells in aggregates, showing significantly higher values in BHS-M and BHS-L compared with cell spheroid and BHS-S. Two-way ANOVA is performed with Tukey's post-hoc multiple comparisons. NS=not significant with p≥0.05, **p<0.01, ****p<0.0001. For comparing days 7 with 1 of each sample, ns=not significant with p≥0.05, ##p<0.01, ####p<0.0001.

FIG. 48 is R_{eq} heatmap of different aggregates during the initial 24 h.

FIG. 49 is R_{eq} heatmap of aggregates from day 1 to 5.

FIG. 50 is a graph showing optical microscopy images of cell spheroids (top block) or BHS-M (bottom block), each comprising HUVEC, MSC, or HUVEC+MSC in the top, middle, and bottom rows, respectively, over five days.

FIG. 51 is a graph showing R_{eq} measured at day 1, comparing cell spheroids or BHS-M, for HUVEC, MSC, and HUVEC+MSC.

FIG. 52 is a graph showing R_{eq} measured at day 3, comparing cell spheroids or BHS-M, for HUVEC, MSC, and HUVEC+MSC.

FIG. 53 is a graph showing R_{eq} measured at day 5, comparing cell spheroids or BHS-M, for HUVEC, MSC, and HUVEC+MSC.

FIG. 54 shows schematic illustrations and fluorescence images of building blocks, including cells and microgels, cell spheroids, cell spheroids and microgels, or BHS, cultured for 72 h to form tissue-like constructs. Upon transferring and gentle pipetting, only the cell spheroids or BHS maintain their structural integrity and cohesion, while the other two groups disintegrate (left)), and further shows radar plots, comparing the advantages of BHS as building blocks for the in vitro fabrication of tissue-like structures based on cell-matrix interactions, scalability, structural integrity, cell viability and activity, modularity, and building block fusion (right).

### DETAILED DESCRIPTION OF THE INVENTION

The following description is of embodiments presently contemplated for carrying out the present invention. This description is not to be taken in a limiting sense but is made merely for the purpose of describing the general principles and features of the present invention. The scope of the present invention should be determined with reference to the claims.

Embodiments relate to bioactive granular hydrogel scaffolds configured for use in tissue engineering and three-dimensional (3D) bioprinting. For example, scaffolds may mimic the mechanical and biological properties of tissues such that, once a scaffold is integrated with host tissue, the scaffold may provide a supportive environment to the tissue. More specifically, cells can migrate from surrounding host tissue into the scaffold. Once cells are adhered to or otherwise integrated with the scaffold, the cells may proliferate and enable tissue growth or regeneration.

Granular hydrogel scaffolds may be formed from jamming (e.g., packing) hydrogel microparticles, followed by crosslinking the hydrogel microparticles through covalent and/or noncovalent intermediate bond formation. The hydrogel microparticles may therefore serve as the building blocks of the scaffolds. The terms “hydrogel microparticles” and “microgels” may be used interchangeably herein. The terms “granular hydrogel scaffolds” and “scaffolds” may similarly be used interchangeably herein.

The microgels may be formed by crosslinking one or more polymers or lipids. Polymers may be selected from the group consisting of proteins, peptides, carbohydrates, or any other natural, semi-natural, or synthetic polymeric materials, and mixtures thereof. In some embodiments, polymers may be selected from the group consisting of hyaluronic acid (HA), polyethylene glycol (PEG), gelatin methacryloyl (GelMA), and mixtures thereof.

In some embodiments, the microgels may be formed from a polymer blend. As used herein, the term “polymer blend” may refer to a homogeneous mixture of two or more polymers, such as a physical mixture of two or more polymers held together by intermolecular forces, without chemical bonds linking therebetween. In some embodiments, a polymer blend may include two or more polymers selected from the group consisting of HA, PEG, and GelMA.

In some embodiments, the polymers may be crosslinked to form the microgels via physical crosslinking and/or chemical crosslinking. Examples of chemical crosslinking include free radical polymerization (e.g., free radical polymerization of vinyl groups) or any other suitable chemical crosslinking techniques.

In some embodiments, the microgels may be nonporous, such that the microgels may not include significant voids or pores in their structures.

In some embodiments, the microgels may be porous, such that voids are incorporated into the structures. As the microgels serve as the building blocks of scaffolds, porous microgels may impart (or increase) porosity to the scaffold itself and may enhance the void fraction of the scaffold compared with scaffolds formed from nonporous microgels.

In some embodiments, the microgel voids may be between 5 and 40 μm.

As used herein, the term “void fraction” refers to a measurement used to quantify the fraction of the total microgel volume that is occupied by voids (or empty spaces).

Voids formed within scaffolds may advantageously promote cell infiltration and host tissue integration. While the use of nonporous microgels may enable inter-microgel pores (e.g., voids between microgels), the void fraction of the resulting scaffold is limited to that of random packing of the microgels. Accordingly, porous microgels may not only attain inter-microgel pores, but also necessarily provide intra-microgel pores (e.g., voids within the microgels themselves), thereby increasing the void fraction of the resulting scaffold. Cell infiltration and host tissue integration may therefore be increased. Further, cell distribution within the scaffolds may similarly be enhanced, thus leading to a more unfirm scaffold in comparison to scaffolds formed from nonporous microgels.

In some embodiments, the microgels and/or scaffolds may have a void fraction between 15 and 60%. Void fractions above 60% may inhibit mechanical strength of the resulting scaffold, while void fractions below 15% may not enable improved cell infiltration and host tissue integration, among other benefits.

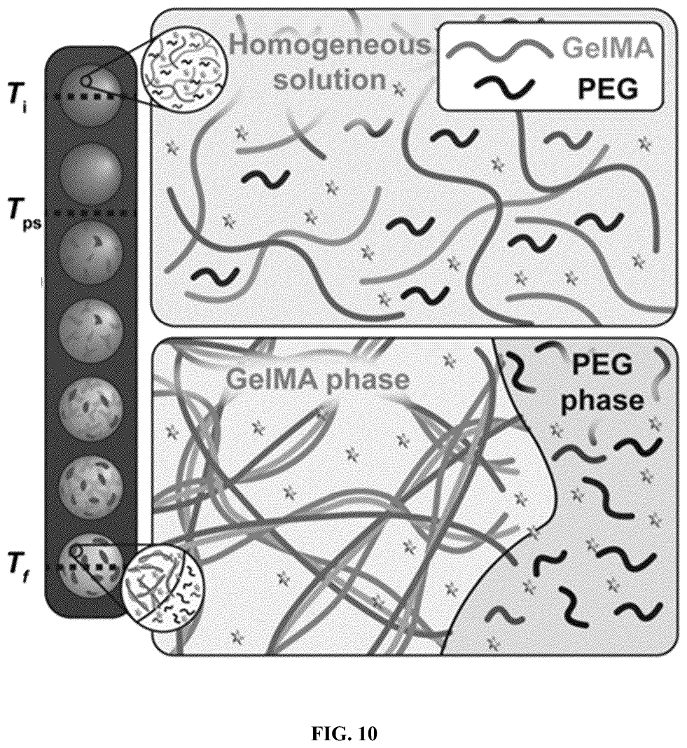

Porous microgels may be formed using any suitable technique. Referring to FIGS. 10-12, in one exemplary method of forming porous microgels, at least a first polymer and a second polymer may be crosslinked and provided as a composite microgel suspension in a liquid solution at an initial temperature. The composite microgel suspension may be thermally phase separated into two distinct phases by lowering the temperature of the suspension to a final temperature, in which the final temperature is lower than the phase separation temperature of the polymer mixture. As used herein, the term “phase separation temperature” refers to a temperature in which a first polymer and a second polymer exhibit a reduction in miscibility and yield two distinct phases, such that the second polymer may be fully or partially separated from the first polymer.

In particular, the thermodynamic incompatibility between the first polymer and the second polymer may lead to liquid-liquid phase separation The microgel suspension may then be filtered from the liquid solution, after which the second polymer may diffuse out of the microgels, resulting in pores within the individual microgels composed of the first polymer. Porous microgels may therefore be provided via thermally induced polymer phase separation.

In some embodiments, the first polymer is GelMA and the second polymer is PEG, such that the resulting porous microgels comprise GelMA after PEG release.

The microgels may be any shape, size, and/or aspect ratio. In some embodiments, at least a portion of the microgels have a spherical shape. In some embodiments, at least a portion of the microgels have a rod-like shape. In some embodiments, the size of the microgels is between 10-200 μm. In a preferred embodiment, the microgels may have an aspect ratio between 1-10.

In some embodiments, the microgels may be crosslinked to form the scaffolds. For example, the scaffolds may be formed via free radical photopolymerization of jammed porous microgels after exposure to light, forming intra-and/or inter-microgel covalent bonds.

In alternative embodiments, the scaffolds may not require light exposure for scaffold formation and may be formed inside of tissues that do not have access to light. These embodiments may be advantageous as they may allow for noninvasive or minimally invasive tissue regeneration, vascularization inducement, axonogensis inducement, and/or tissue function improvement techniques using the scaffolds without requiring open surgery.

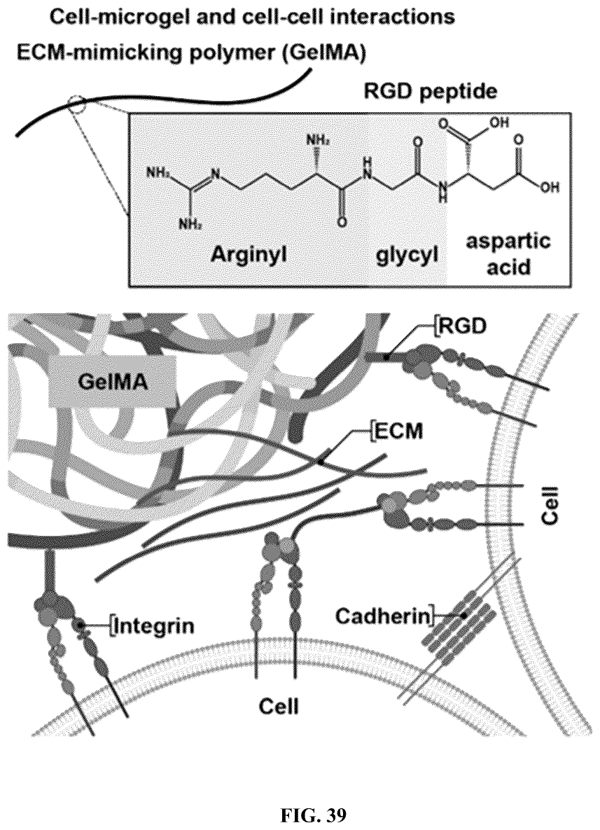

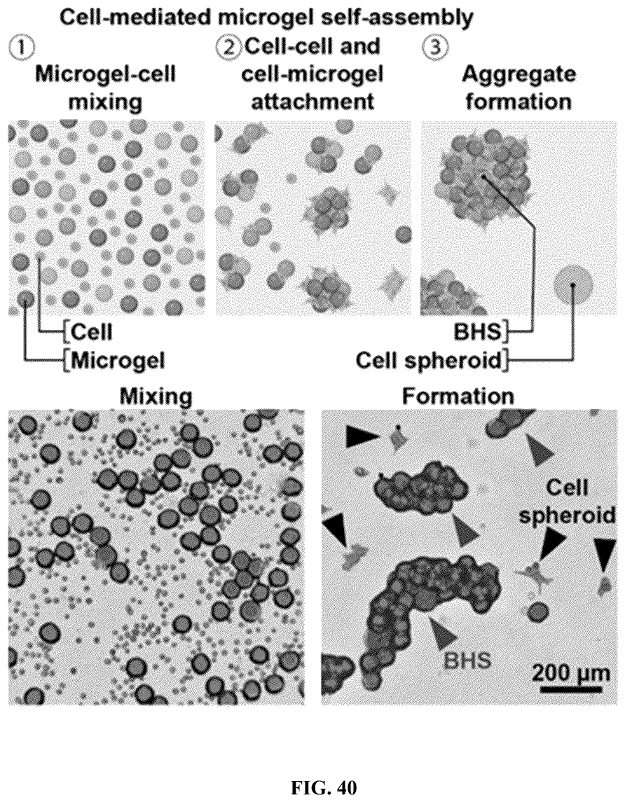

In some embodiments, the microgels may be combined with adherent cells to form hybrid (e.g., cell-microgel) aggregates. In particular, referring to FIGS. 39 and 40, the cells may serve as assembly engines and migrate/adhere to the microgels such that a self-assembly process is initiated and aggregates are formed. In particular, microgels may be significantly larger (≥5 times) than the cells such that porous aggregates are formed. Porous aggregates may enhance molecular diffusion and improve cell viability. Such aggregates may be scalable and be used to self-assemble large-scale physiologically relevant tissue models in vitro.

In some embodiments, the porous aggregates may have a void fraction between 3 and 30%.

In some embodiments, the microgels may be combined with cells on a planar non-adhesive substrate to form the hybrid aggregates. The substrate may prompt cell-cell and cell-microgel interactions.

In some embodiments, combining the cells and microgels may initiate a three step assembly process: (i) initial mixing of cells and microgels, (ii) early-stage attachment of cells to microgels because of the adhesive moieties on the polymer(s), and (iii) the formation of two distinct aggregate types (e.g., hybrid aggregates, and cell aggregates).

In some embodiments, the hybrid aggregates may be crosslinked to form a scaffold. For example, the scaffold may be formed via free radical photopolymerization of the hybrid aggregates.

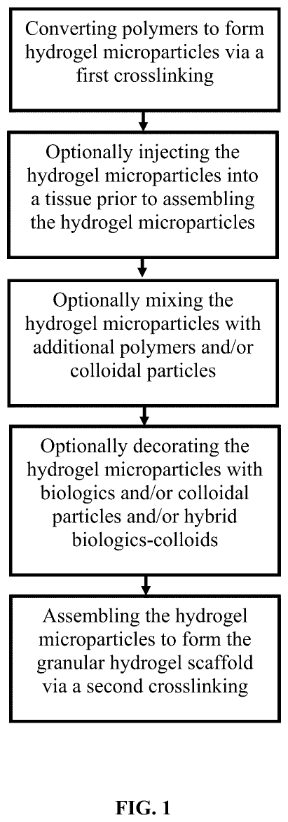

Referring to FIG. 1, embodiments may relate to a method of forming a granular hydrogel scaffold. The method may comprise converting polymers to form hydrogel microparticles (HMP) via crosslinking and assembling the HMP to form scaffolds, such as via free radical photopolymerization or non-light-mediated crosslinking.

In some embodiments, the HMP are injected to an injection site within tissue. In such embodiments, the HMP may assemble to form the scaffolds after injection into the tissue via non-light-mediated crosslinking. It is contemplated that this step of crosslinking may be based on crosslinking of functional groups not used to crosslink the polymer to form the HMP, such as amines, using enzymes, dynamic covalent bond formation, or any other suitable crosslinking technique.

It is further contemplated that the HMP may assemble to form scaffolds after mixing with another polymer and/or colloidal particles. It is contemplated that this polymer may be aldehyde-modified carbohydrates and/or proteoglycans, including hyaluronic acid, protein and/or polymers in the extracellular matrix of native tissues, or another other suitable polymer that may form a hybridized scaffolds.

It is further contemplated that the HMP may assemble to form scaffolds after being decorated with biologics and/or colloidal particles and/or hybrid biologics-colloids. The surface of the HMP may be coated with any biologics and/or the biologics may be encapsulated in the HMP. The biologics may be biomolecules, growth factors (e.g., of hematopoietic growth factors, EGF, FGF, NGF, PDGF, VEGF, IGF, GMCSF, GCSF, TGF, Erythropieitn, TPO, BMP, HGF, GDF, Neurotrophins, MSF, SGF, and GDF and any other growth factors or biomacromolecules), cytokines, enzymatically modified DNA, drugs, peptides, or any combination thereof, or any other suitable biologics that may form scaffolds with enhanced bioactivity (e.g., a bioactive scaffolds). In exemplary embodiments, the biologics may be loaded (i.e., conjugated) to, attached on the surface of, or hybridized with nanocarriers bearing crosslinkable functional groups (e.g., vinyl groups). In exemplary embodiments, the biologics (e.g., the growth factors) are physically and/or chemically attached to colloids (e.g., heparin nanoparticles).

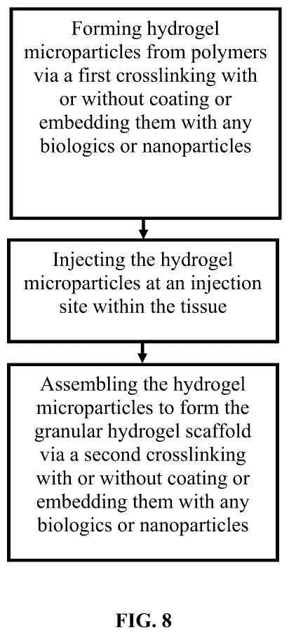

Referring to FIG. 8, embodiments relate to a method of regenerating tissue, inducing vascularization, inducing axonogenesis, and/or improving tissue function via a granular hydrogel scaffold. The method comprises forming HMP derived from polymers via crosslinking, injecting the HMP at an injection site within the tissue, and assembling the HMP to form the scaffolds via non-light-mediated crosslinking. It is contemplated that the scaffolds may be configured to mimic the physiochemical and/or biological characteristics of the tissue. In particular, the scaffolds may be configured to mimic the stiffness of the tissue.

In exemplary embodiments, the tissue is selected from the group consisting of soft and/or hard tissues, including nervous tissue (brain, spinal cord, nerves), endothelial tissue, epithelial tissue (skin, GI tract), muscle tissue (cardiac muscle, smooth muscle, skeletal muscle), and/or connective tissue (fat, bone, tendon, cartilage).

### EXAMPLES

### Example 1

### Materials

Gelatin type A (Sigma-Aldrich, USA), Dulbecco's phosphate buffered saline (DPBS) (Sigma-Aldrich, USA), methacrylic anhydride (Sigma-Aldrich, USA), and dialysis membranes (cutoff M_{w}=12-14kDa, Spectrumlabs, USA) were used for GelMA synthesis. Lithium phenyl-2,4,6-trimethylbenzoylphosphinate (LAP) (Allevi, USA), (4-(2-hydrozyethyl)-1-piperazineethanesulfonic acid (HEPES) (Gibco, USA), Picosurf (Sphere Fluidics Inc, UK), Novec 7500 (3M, USA), and perfluorooctanol (PFO) (Sigma-Aldrich, USA) were used for GelMA HMP fabrication. Ca²⁺ (Thermo scientific, USA), thrombin (EMD Millipore, USA), and Factor XIII (FXIII) (EMD Millipore, USA) were used for the assembly of GelMA HMP to form GHS. Fluorescein isothiocyanate (FITC) (Sigma-Aldrich, USA) was used for labeling the void spaces of GelMA GHS.

### Methods—GelMA Synthesis

GelMA has been synthesized in the following manner. Briefly, 10% w/v gelatin type A was dissolved in 50° C. DPBS and reacted with methacrylic anhydride 1.25% (v/v) for 2 h. The reaction was stopped by adding DPBS (twice the reaction volume). The GelMA solution was dialyzed against 40° C. miliQ water using dialysis membranes (cutoff M_{w}=12-14kDa) for 7 days. The GelMA solution was then filtered and frozen for 3 days at −80° C., followed by freeze-drying at 0.12 mbar. The lyophilized GelMA was stored at 2-8° C. until further use.

### Methods—GelMA Stiffness Optimization

Lyophilized GelMA was dissolved in the HEPES buffer (25 mM, pH=7.2-7.4) containing LAP (0.1% w/v). The final concentration of GelMA solution was 1, 1.5, 2, or 3% w/v. The GelMA solutions were transferred to a mold and stored at 2° C. overnight to physically crosslink, followed by UV light (wavelength=395 nm) exposure at an intensity of 15 mW cm⁻² for 30 or 60 s. The photocrosslinked GelMA was punched with an 8 mm biopsy puncher and transferred to a rheometer (TA instrument, USA) to perform frequency sweep tests at a strain rate of 0.1% and angular frequencies of 0.1 to 100 rad s⁻¹. As a proof-of-concept, the average value of photocrosslinked GelMA storage modulus (G′) at 1 rad s⁻¹ was assessed to optimize the GelMA stiffness/concentration for brain studies. This disclosure is valid for any concentration of GelMA or any other protein/peptide/biopolymer.

### Methods—GelMA HMP Fabrication and Stabilization

The GelMA droplet formation may be conducted using a step emulsification or any other device or bulk emulsification. Briefly, a GelMA solution at the optimum polymer concentration that would yield the target tissue stiffness after crosslinking (e.g., 1.5% w/v to mimic brain) was dissolved in the HEPES buffer containing LAP (0.1% w/v). To form GelMA droplets in an oil phase, the GelMA solution was flowed into a microfluidic device as a dispersed aqueous phase, and an oil containing Picosurf 2% (v/v) in Novec 7500 was used as the continuous phase. The GelMA droplets were stored at 2-4° C. to undergo physical crosslinking.

After collecting the GelMA droplets, the emulsion was broken using a PFO solution in Novec 7500 oil (20% w/v). In addition, the microgels were mixed and centrifuged with the solution of 0.1% (w/v) LAP in HEPES to completely remove the oil. The washed microgels were spun down and suspended in a solution of 0.1% (w/v) LAP in HEPES at a concentration of 10% (v/v). Before photocrosslinking, the microgels were always maintained at 4° C. The diluted microgels were placed on a stirrer, and vigorously stirred while being exposed to the UV light (wavelength=395 nm, GearLight, USA) for 60 s at an intensity of 15 mW/cm². The UV exposure initiates the vinyl group-enabled covalent bond formation of GelMA. In this process, other types of crosslinkers, such as other photoinitiators and/or chemical agents (dithiothreitol, DTT) may also be used to stabilize the individual microgels. If the microgels are not stabilized, they immediately dissolve at 37° C. As the GelMA bears vinyl groups, thiol-based crosslinkers, such as DTT, can enable microgel stabilization via the Michael-type reaction between the vinyl and thiol groups. The GelMA within the individual HMP can also be crosslinked using click chemistry by modifying the GelMA carboxyl groups with pendant norbornene and tetrazine. Finally, the crosslinked microgels were stored in HEPES containing 10 mM of Ca²⁺. To assess the stability of microgels, they were transferred to an incubator and imaged over time at 37° C. At timepoints of 0, 0.5, 1, 3, and 24 h, the microgels were imaged using a brightfield microscope. The microgel diameter was analyzed using a custom-written MATLAB code identifying the HMP border. Thermally stable microgels did not undergo significant size change over time.

### Methods—GelMA HMP Modification

To enhance the bioactivity of GHS, embodiments enable the direct modification of GelMA HMP with biomolecules, growth factors (such as VEGF, SGF, and BMP), cytokines, enzymatically modified DNA, drugs, and/or peptides. These modifications can be conducted via GelMA HMP surface modification or encapsulation. In addition, the biologics, such as VEGF, can be loaded/conjugated to nanocarriers, such as heparin nanoparticles (nanoheparin, nH) bearing crosslinkable functional groups, such as vinyl groups. In this case, the growth factor-loaded nH can be directly conjugated to the surface of GelMA HMP through chemical (e.g., covalent) bond formation. The GelMA HMP surface may be coated with any biologics and/or the biologics may be encapsulated in the GelMA HMP. The modified HMP will remain annealable and form composite/nanocomposite GHS, similar to the unmodified HMP.

### Methods—GelMA GHS Formation

The individually crosslinked GelMA HMP were incubated in the HEPES buffer containing 10 mM of Ca²⁺ at room temperature, followed by packing at 14,000 rpm for 5 min. After centrifugation, the supernatant was removed and the excess water among the microgels was removed using a Kimwipe. The packed microgels were aliquoted to microcentrifuge tubes, each of which containing 88 μL of packed GelMA HMP. A solution of thrombin with a concentration of 2 U/mL in HEPES containing Ca²⁺ (10 mM) and a solution of FXIII with a concentration of 10 U/mL in HEPES containing Ca²⁺ (10 mM) were added to two separate aliquoted HMP tubes. Each GelMA HMP aliquot was well mixed with their respective biomacromolecule solutions (FXIII or thrombin), followed by pulse centrifugation. Then, the microgel assembly (GHS formation) was initiated by mixing an equal volume of thrombin-containing microgels and FXIII-containing microgels. Upon mixing, thrombin and calcium activate FXIII, yielding FXIIIa. The two microgel suspensions were mixed using a positive displacement pipette (Gilson, USA). The mixture was spun down at 14,000 rpm, and the supernatant was removed. The mixed microgel was transferred to a mold using a positive displacement pipet and incubated at 37° C. to initiate the FXIIIa-mediated formation of glutamyl-lysine bonds among HMP. In this system, the lysine and glutamine peptides were used to form the glutamyl-lysine bonds with the enzymatic reaction. In addition, based on the target tissue and application, other types of crosslinkers, such as glutaraldehyde, can perform a Schiff-base reaction between lysine and aldehyde groups. GelMA GHS may also be hybridized with other polymers, such as those in the extracellular matrix (ECM) of native tissues. As an example, polysaccharides such as hyaluronic acid (HA) or alginate, were modified with the aldehyde groups to form a Schiff base with protein HMP, such as GelMA. This is an important aspect of the disclosed subject matter, eliminating the necessity of using enzymes, such as thrombin or FXIII, which are expensive, easy to degrade, and often bioactive. Such mechanism can be generalized to other peptides and crosslinkers.

### Methods—GelMA GHS Pore Characterization

To analyze the interconnected void spaces of GelMA GHS, the assembled scaffolds were assessed after 90 min of enzymatic assembly at 37° C. They were incubated with a FITC (M_{w}=0.5 MDa) solution (15 mM) for 5 min. The labelled scaffold was imaged using a fluorescence microscope (Leica DMI8, Germany). For each sample (at least 3 per condition), the z-stack images were acquired to analyze at least 3 layers of packed microgels within the scaffold. To analyze the scaffold porosity, the void fraction was calculated by adjusting the threshold of images using the Leica LAS X software. In addition, the median pore diameter of scaffolds was calculated by analyzing the equivalent area of individual pores using a custom-written MATLAB code (MATLAB 2021, USA).

### Methods—Mechanical Characterization of GelMA GHS

The assembled GHS (height˜1 mm) after 90 min of incubation at 37° C. were incubated in room temperature HEPES for 1 h. The scaffolds were punched with 8 mm biopsy puncher, placed on the Instron 5943 (Norwood, MA, USA) lower plate, and underwent compression while the force was measured using a 10 N load cell. The force-displacement data were converted to stress-strain curves. The compression tests were performed at a displacement rate of 1 mm min⁻¹. The compressive modulus of GHS was calculated based on the linear stress-strain region at strain˜0.05-0.15 mm mm⁻¹. At least 5 scaffolds per condition were analyzed for mechanical characterizations.

### Methods—Statistical Analysis

The one-way analysis of variance (ANOVA) was performed for statistical analysis, and statistically significant differences were identified when p-values were lower than 0.05 (*p <0.05), 0.01 (**p<0.01), 0.001 (***p <0.001), and 0.0001 (****p<0.0001).

### Results and Discussion

### GelMA GHS Formation

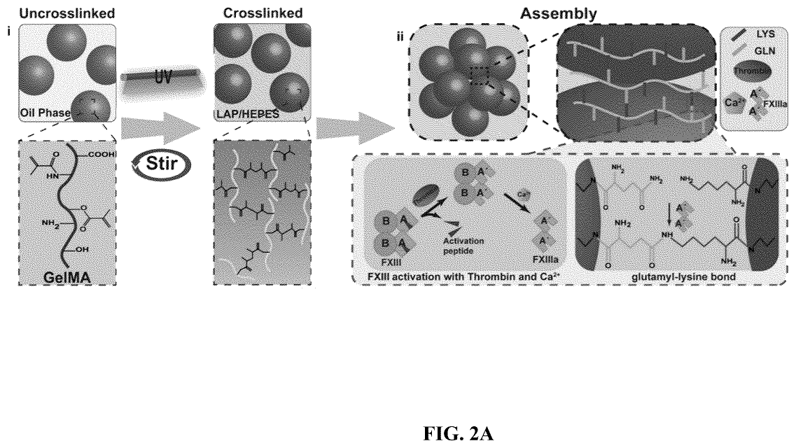

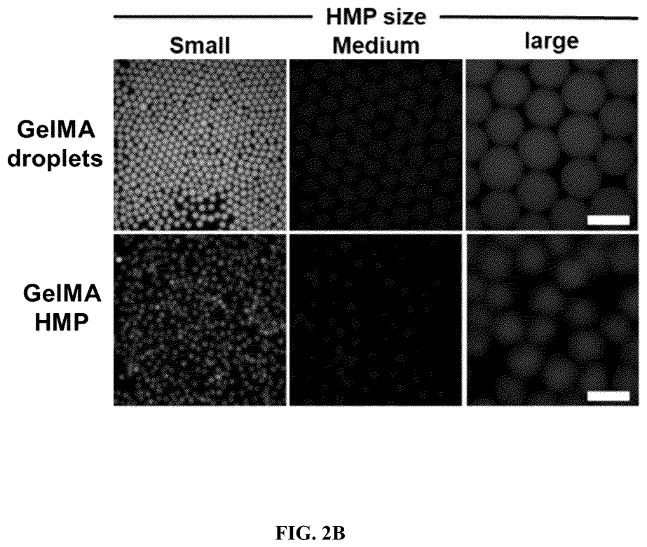

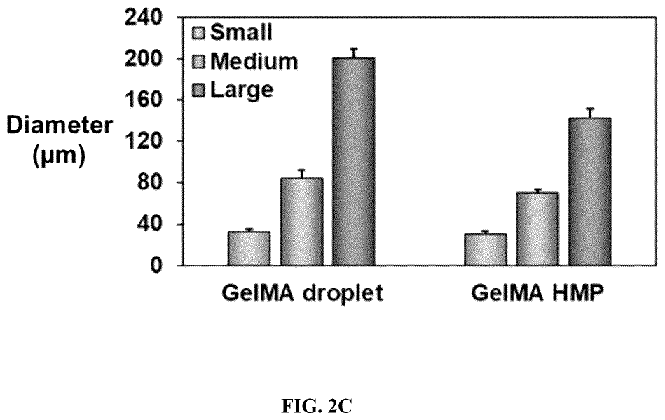

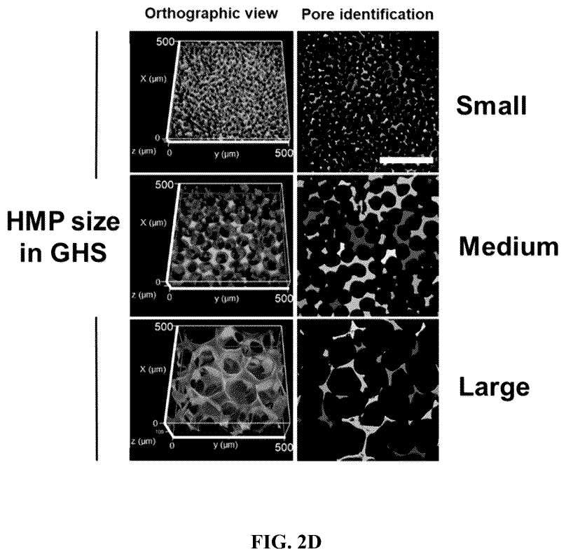

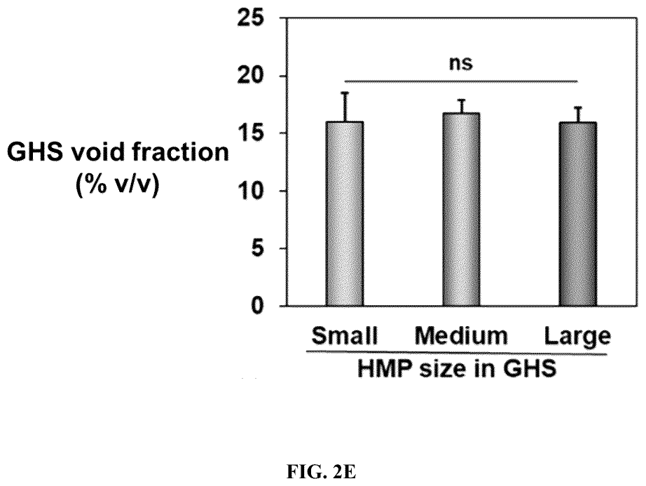

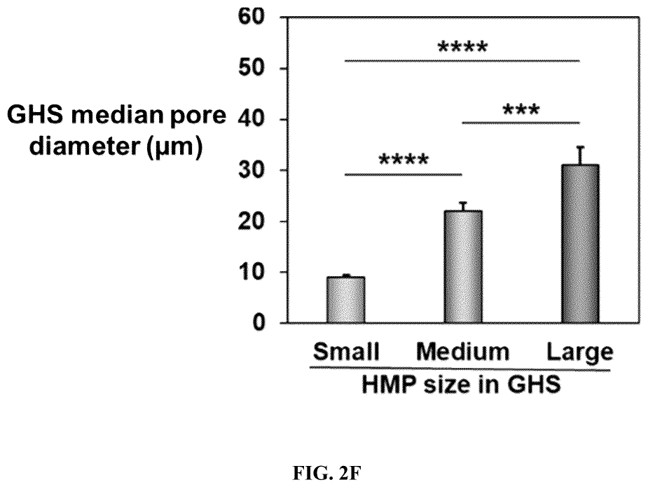

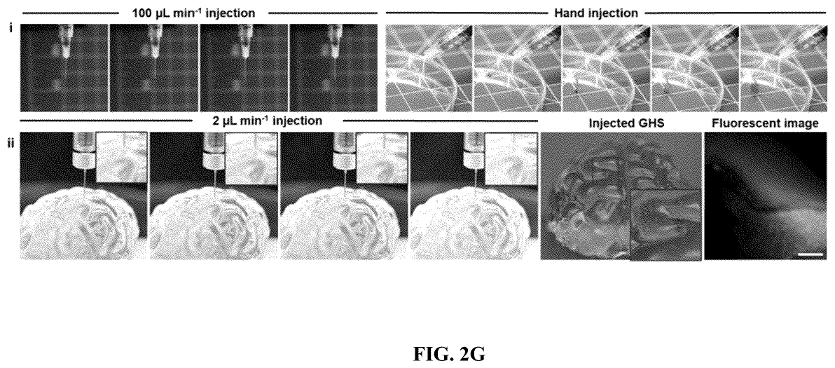

A multi-step procedure to first chemically stabilize HMP and then assemble them to form GHS is provided. GelMA HMP are first physically crosslinked at 2-4° C., followed by UV light (e.g., wavelength˜395 nm and intensity˜15 mW cm⁻² for 60 s) exposure in a dilute suspension of HEPES, containing LAP (0.1%, w/v) at 4° C. Throughout the UV exposure process, GelMA precursor within each HMP is chemically crosslinked via free radical polymerization, as schematically shown in FIG. 2A. GelMA contains various peptides, such as lysine (Lys) and glutamine (Gln). The biocompatible assembly of UV crosslinked GelMA HMP to form GelMA GHS is conducted via the enzymatic formation of glutamyl-lysine bonds. FXIII, also known as fibrin stabilizing factor, is a zymogen (an inactive material that is converted to an enzyme upon activation) found in human blood. Thrombin can activate FXIII to form activated FXIII (FXIIIa), which is an enzyme responsible for the crosslinking of fibrin during the blood coagulation cascade. FXIIIa catalyzes the formation of ε-(γ-glutamyl) lysine isopeptide bonds between the ε-amino groups of Lys residues (K peptide, donor) and γ-carboxamide groups of Gln residues (Q peptide, acceptor) of gelatin, similar to those of fibrin monomers.

FIG. 2A schematically presents the mechanism of GelMA GHS formation via the FXIIIa mediated glutamyl-lysine bond formation among the individual HMP. Accordingly, the GelMA HMP undergoes microgel-microgel assembly, catalyzed by the FXIIIa at 37° C., forming GelMA GHS.

The pore characteristics of GHS directly regulate the scaffold-cell interactions, cell recruitment, foreign body response, and tissue regeneration. Accordingly, it is important to tailor the void space within GHS. The pore features of GHS are tuned by varying the size of HMP building blocks, as the pore size is regulated by the void space among the microgels. To engineer the pore size of GHS, various GelMA HMPs were fabricated with an average diameter of 31.9±3.2, 85.5±6.0, or 164.8±14.6 μm, labeled as small, medium, or large, respectively (see FIGS. 2B and 2C).

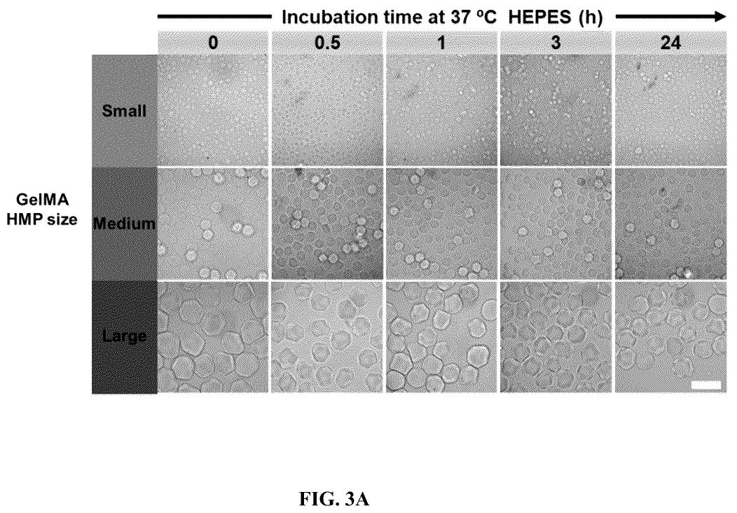

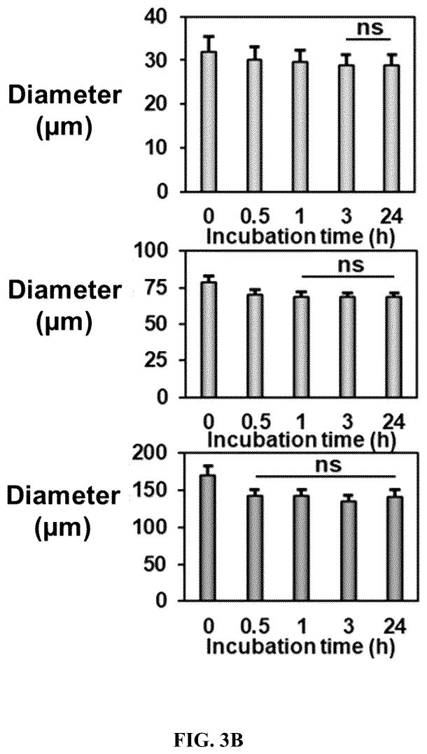

FIGS. 3A and 3B shows the size change of photocrosslinked GelMA HMP at 37° C. As can be seen in these figures, the photocrosslinked GelMA HMP are stable at the physiological temperature (before assembly). Finally, the GelMA GHS were fabricated by mixing the GelMA HMP with the FXIIIa, followed by packing and incubation at 37° C. for 90 min.

To demonstrate the interconnectivity of pores and analyze the pore features of GelMA GHS, the scaffolds were incubated with the FITC-dextran fluorescent dye. FIG. 2D shows the fluorescently labeled GelMA GHS, fabricated from three groups of GelMA HMP (small, medium, and large) as well as the identified pores using a custom-written MATLAB code.

FIG. 2E shows the analysis of GelMA GHS void fraction, which suggests that the void fraction is independent of microgel size, and the average void fraction is ˜16.0±2.4% (v/v). In addition, the median pore size of GelMA GHS was measured at a z-stack wherein the microgels have the largest contact with each other. The images were analyzed using the MATLAB code to detect the area of individual pores and calculate the equivalent pore diameter. As presented in FIG. 2E, the median pore diameter of GelMA GHS fabricated from the small, medium, and large HMP is ˜8.9±0.4, 21.9±1.6, and 30.9±3.5 μm, respectively. These results show that although the void fraction is similar in all three groups of GHS as a result of spherical granules, the pore size significantly increases by increasing the microgel size.

FIG. 2G shows the injectability of packed GelMA HMP through 30G needles using a syringe pump or by hand, mimicking the brain injection process to facilitate tissue regeneration after stroke. FIG. 2G also presents the HMP injection via a 30G needle (Small Hub RN Needle, Hamilton, USA) connected to a 5 μL syringe (Hamilton, USA) using a syringe pump at a rate of 2 μL min⁻¹. As can be seen in FIG. 2G, the GelMA HMP is injectable via the clinically relevant needles, enabling scaffold formation in a lesion after injection.

### Mechanical Characterization of GelMA HMP and GHS

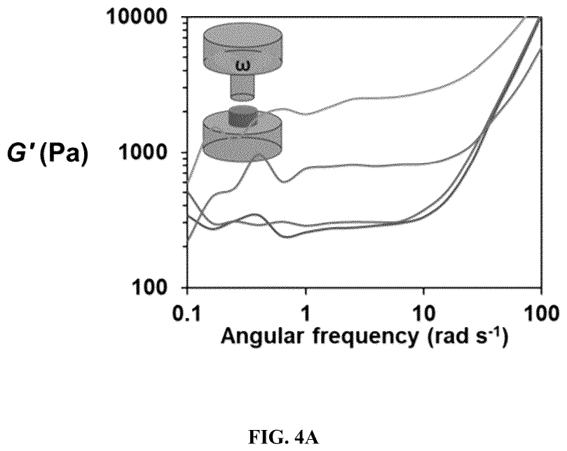

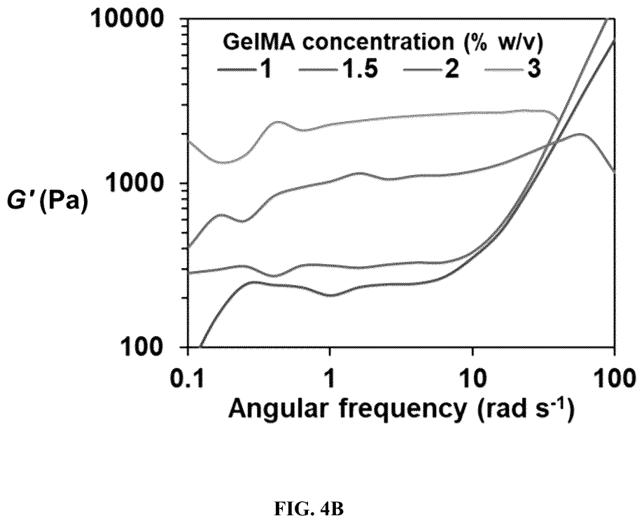

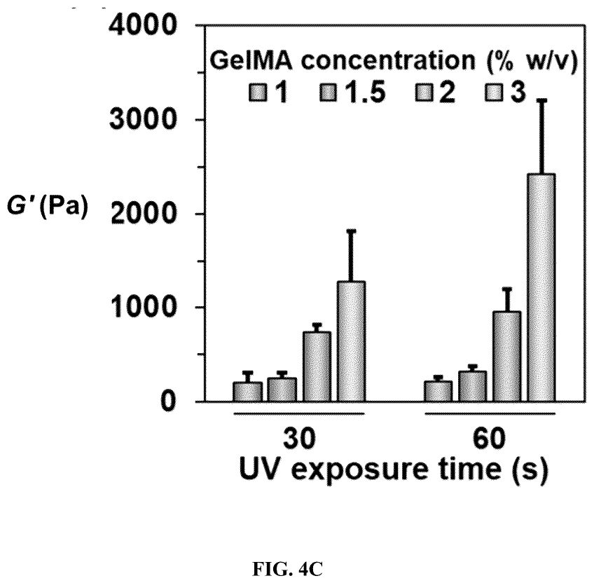

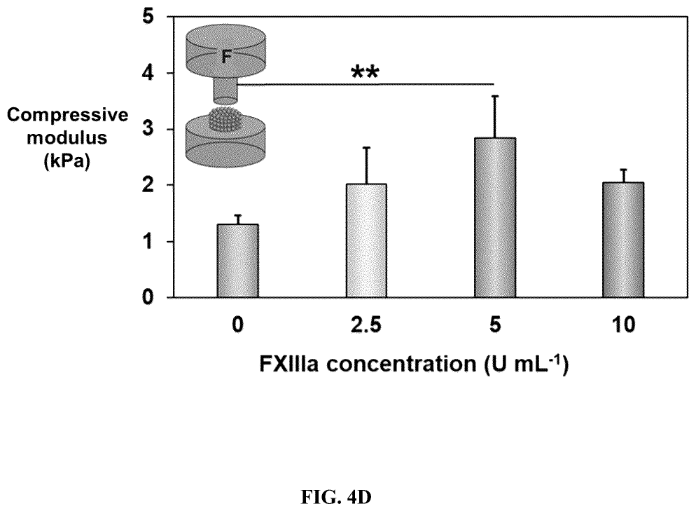

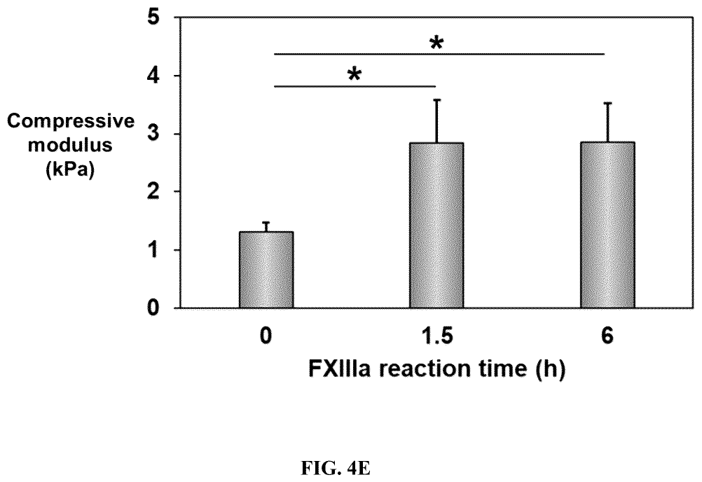

The mechanical properties of GelMA should be optimized to mimic target tissues. As an example, to match the stiffness of brain tissue, the stiffness of GelMA was tailored via changing the GelMA concentration and photocrosslinking time. Varying GelMA concentrations (1, 1.5, 2, or 3% w/v in HEPES, containing 0.1% LAP) were used to prepare physically crosslinked bulk samples maintained at 2-4° C. overnight, followed by UV light exposure for 30 s or 60 s at an intensity of 15 mW cm^{-2.} The representative storage modulus graphs are shown in FIGS. 4A and 4B for these exposure times, respectively.

FIG. 4C presents the average and standard deviation of bulk GelMA storage modulus at varying GelMA concentration and photocrosslinking times. The higher the biopolymer concentration or UV exposure time, the higher the storage modulus within the experimental range. The target average value of storage modulus at a frequency of 1 rad s⁻¹ is the native rat brain modulus of ˜330 Pa. The average storage modulus of the GelMA with a concentration of 1.5% (w/v) and the UV curing time of 60 s was around 325 Pa, which has a good agreement with the native brain tissue (FIG. 4C). Note that the stiffness of the bulk samples represents the stiffness of individual GelMA HMP.

The HMP assembly process was initiated by mixing the GelMA HMP with FXIIIa, followed by incubation at 37° C. To determine the stiffness of GelMA GHS, compression tests were performed on the GHS. The concentration of the FXIIIa was optimized based on the compressive modulus of assembled scaffolds, compared with packed unassembled ones (the control group).

FIG. 4D shows the compressive modulus of unassembled (FXIIIa concentration=0) and assembled GHS with the FXIIIa concentration of 2.5, 5, and 10 U mL⁻¹. The average compressive modulus of unassembled GHS (i.e., packed HMP) was around 1.31±0.15 kPa, and that of assembled GHS was 2.02±0.64, 2.83±0.74, and 2.05±0.22 kPa for the FXIIIa concentrations of 2.5, 5, and 10 U mL⁻¹, respectively. Accordingly, FXIIIa with a concentration of 5 U mL⁻¹, obtained from activating FXIII using thrombin (1 U mL⁻¹), resulted in GHS stiffness that was significantly higher than the control group (no FXIII), which will be used as the optimum enzyme concentrations for the GHS formation (see FIG. 4D).

FIG. 4E shows the compressive modulus of GelMA GHS formed at two different FXIIIa reaction times (1.5 and 6 h) compared with the packed HMP that did not undergo the FXIIIa reaction. The compressive modulus of assembled GHS at the FXIIIa concentration of 5 U mL⁻¹ was around 2.83±0.74 or 2.36±0.96 kPa for the GHS incubation time of 1.5 or 6 h at 37° C., respectively. These results show that the scaffold stiffness reaches a plateau after 1.5 h of incubation at 37° C. Therefore, the scaffolds are less likely to undergo further stiffening after 1.5 h post injection in tissues, such as brain.

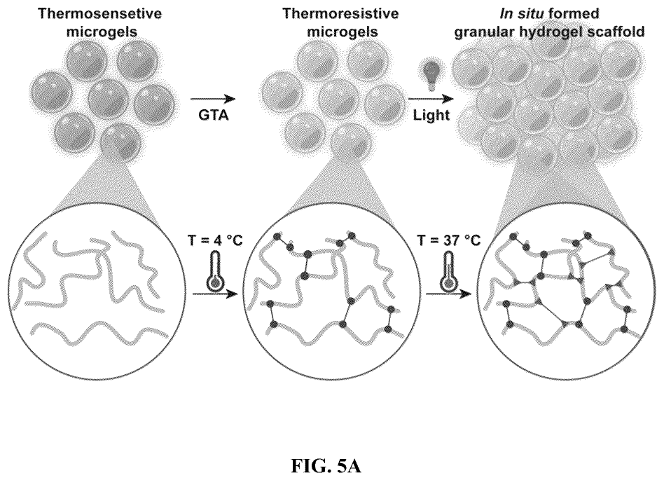

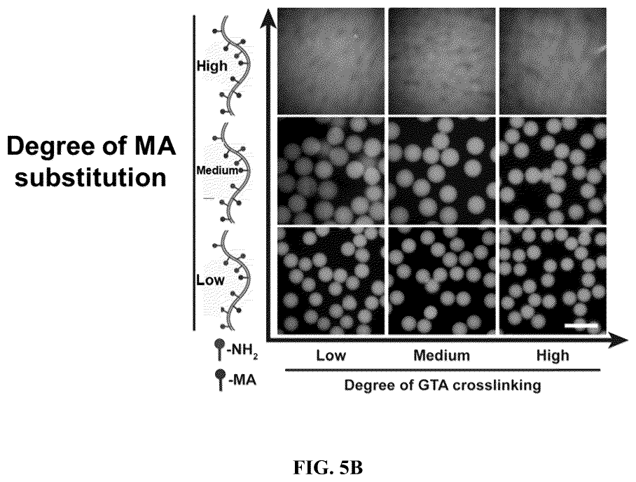

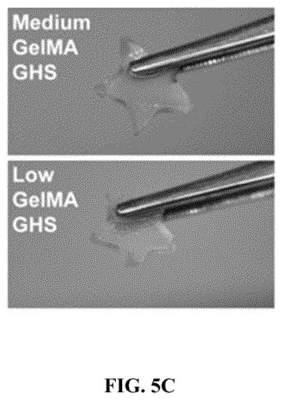

FIGS. 5A-C show in situ formation of GHS from thermoresistive GelMA microgels. FIG. 5A shows that GelMA HMP are thermoresponsive and unable to form a scaffold at body temperature (e.g., 37° C.). To overcome this limitation, the GelMA HMP was crosslinked via a Schiff-base reaction to produce thermoresistant microgels. These thermoresistive microgels served as building blocks for the in situ fabrication of GHS via radical photopolymerization.

FIG. 5B shows that GelMA was synthesized using three different degrees of substitutions (DOS) with high (˜70%), medium (˜40%) and low (˜10%), which affected the availability of primary amine groups for Schiff-base crosslinking using glutaraldehyde (GTA). High DoS resulted in insufficient primary amine groups, making the thermoresistive microgel unstable. In contrast, medium and low DoS resulted in stable thermoresistive microgels.

FIG. 5C shows thermoresistive microgels from medium and low GelMA were scaffolded to form a mechanically stable GHS.

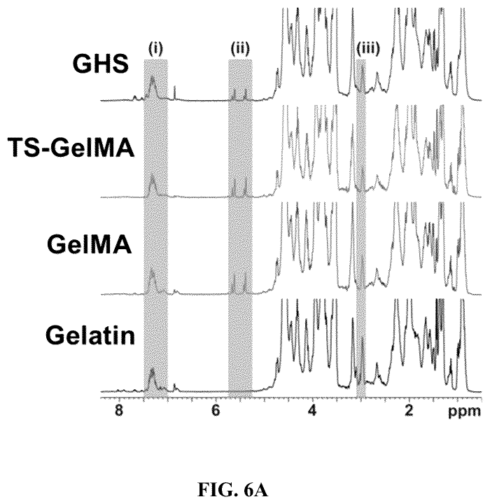

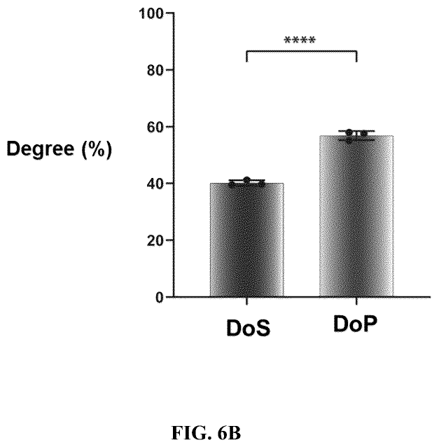

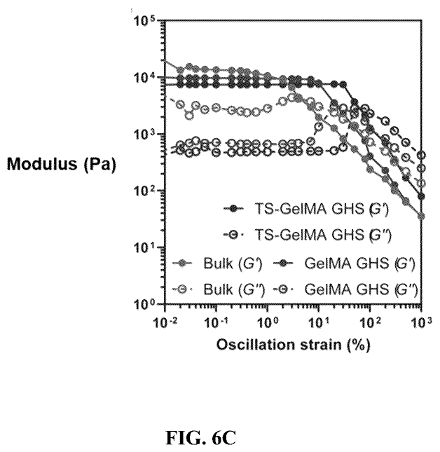

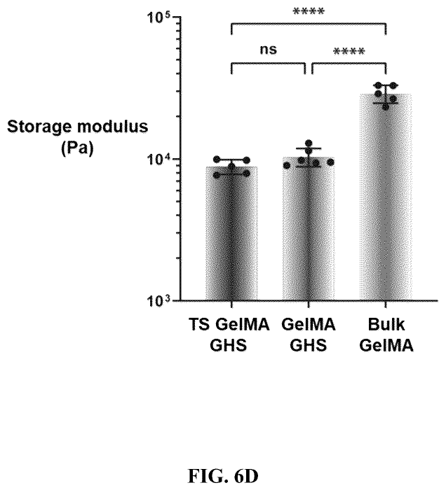

FIGS. 6A-D show material and mechanical characterization of GHS made from thermoresistant microgels. FIG. 6A shows 1H NMR spectrometry of pure gelatin microgel, uncrosslinked GelMA microgel (with medium DoS), thermoresistant GelMA HMP, and photocrosslinked GHS composed of thermoresistant microgels. The peaks for aromatic acids (i) serve as the reference in all the groups. The presence of vinyl groups (ii) and a decrease in lysine protons (iii) were also observed.

FIG. 6B shows oscillatory strain sweep at constant frequency of 1 rad/s performed on in situ fabricated scaffolds: GHS from thermoresistant GelMA microgels (TS GelMA GHS), GHS from thermoresponsive GelMA microgels (GelMA GHS), and conventional (bulk) GelMA scaffolds.

FIG. 6C shows dynamic moduli versus oscillation strain for different scaffolds at a constant frequency of 1 rad/s, showing the storage and loss moduli of TS GelMA GHS, GelMA GHS, and conventional (bulk) GelMA scaffolds.

FIG. 6D shows the storage modulus measured at 1 rad/s and 0.1% strain. No significant difference was observed between the TS GelMA GHS and GelMA GHS, but bulk GelMA had significantly higher stiffness.

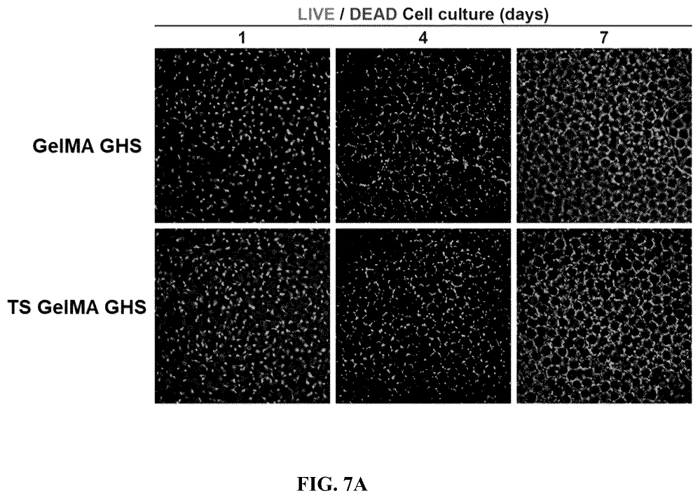

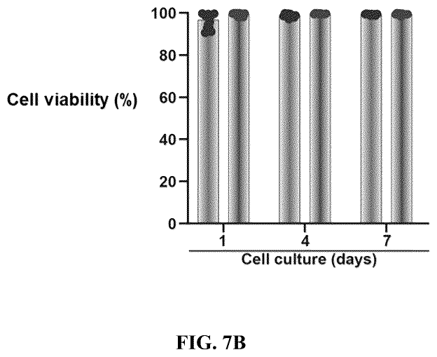

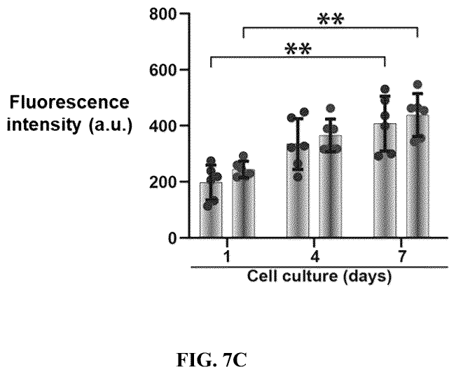

FIGS. 7A-C show biocompatibility assessment of GHS made from thermoresistant microgels. FIG. 7A shows that NIH-3T3 murine fibroblast cells were mixed with thermoresponsive or thermoresistant microgels, then photocrosslinked to form GelMA GHS or TS GelMA GHS, respectively. Fluorescent microscopy using a combination of green (representing live cells) and red (representing dead cells) dyes was performed over a period of 7 days, demonstrating cell proliferation and viability.

FIG. 7B shows the results if cell viability percentage indicated a consistently high viability rate (˜100%) for all GHS samples.

### Example 2: Effect of Porous Microgels

### Materials

Bovine serum albumin (BSA), citrate buffer (pH=6.0), dimethyl sulfoxide (DMSO), Dulbecco's phosphate buffered saline (DPBS, powder), Fluoroshield™ with 4′,6-diamidino-2-phenylindole (DAPI), fluorescein isothiocyanate-dextran (FITC-dextran, average molecular weight=2 MDa, equivalent Stokes diameter˜54 nm), gelatin powder (Type A, ˜300 g Bloom, from porcine skin), lithium phenyl-2,4,6-trimethylbenzoylphosphinate (LAP), methacrylic anhydride, polyethylene glycol (PEG, number average molecular weight=20000), TWEEN® 20, 1H,1H,2H,2H-perfluoro-1-octanol (PFO), and Triton X-100 were purchased from MilliporeSigma, MA, USA. Milli-Q water purification system, used to produce ultrapure water (electrical resistivity˜18.2 MΩ cm at 25° C.), was provided by Millipore Corporation, MA, USA. Spectra/Por 4 dialysis tubing with 12-14 kDa molecular weight cut-off was purchased from Spectrum Laboratories, NJ, USA. Vacuum filtration unit (pore size=0.20 μm), and VWR® Vista Vision™M microscopic slides were purchased from VWR, PA, USA. Novec™ 7500 Engineered Fluid was purchased from 3M, MN, USA. Pico-surf® (2% w/w in Novec™ 7500) was provided by Sphere Fluidics, Cambridge, UK. Fetal bovine serum (FBS) and antibiotic/antimycotic solution (10,000 U mL⁻¹ penicillin G, 10,000 μg mL⁻¹ streptomycin, 25 μg mL⁻¹ amphotericin B) were supplied by Cytiva, MA, USA. DPBS (liquid), phosphate buffered saline (PBS), Dulbecco's Modified Eagle's Medium (DMEM), and trypsin-ethylenediaminetetraacetic acid (EDTA) solution (0.25%) were provided by Gibco, MA, USA. 24 well non-treated plate, T-75 cell culture flasks, and centrifuge tubes (15 mL and 50 mL) were supplied by Celltreat Scientific Products, MA, USA. 96-well flat clear bottom black polystyrene microplates were purchased from Corning, NY, USA. Live/Dead™ cell imaging kit (488/570) containing calcein acetoxymethyl ester (calcein AM) and BOBO-3 iodide, PrestoBlue™ HS (high sensitivity) cell viability reagent, and Alexa Fluor™ 488 phalloidin were provided by Invitrogen, MA, USA. Xylene substitute, Hematoxylin 560™, Alcoholic Eosin Y 515™, Blue Buffer 8™, and Define® were purchased from Leica Biosystems, IL, USA. Recombinant Alexa Fluor 488 anti-cluster of differentiation 31 (CD31) antibody (ab305267) and Alexa Fluor 647 anti-CD68 antibody (ab305214) were purchased from Abcam, CA, USA. Diamond® white glass charged slides, and snap cap 1.7-and 2-mL microcentrifuge tubes were purchased from Globe Scientific, NJ, USA. Alpha-smooth muscle actin (α-SMA) monoclonal antibody (1A4), Alexa Fluor™ 488, eBioscience™ (53-9760-82), and paraformaldehyde (PFA) were purchased from Thermo Fisher Scientific, MA, USA. Isoflurane was provided by Akorn, Inc., IL, USA. Loxicom® meloxicam solution (5 mg mL⁻¹) was purchased from Norbrook Laboratories, KS, USA. Ethanol (200 proof) was provided by Decon Labs, Inc., PA, USA. Chlorhexidine® scrub was supplied by Aspen Veterinary Resources LTD., CO, USA. Surgipath Paraplast X-tra was provided by Leica Biosystems, Germany. Macrosette® processing/embedding cassettes with cover were supplied by Ted Pella Inc., CA, USA. Acrylic sheets were purchased from Astra Products Inc., NY, USA.

### Methods—GelMA Synthesis

GelMA synthesis was carried out based on published protocols. Briefly, 16 mL of methacrylic anhydride was gradually added to 200 mL of a gelatin solution in DPBS (concentration=10% w/v), while being continuously stirred at 50° C. The reaction was terminated after 2 h through the addition of 400 mL of DPBS at 40° C. Subsequently, the solution underwent dialysis against ultrapure water at 40° C. for 10 days using the dialysis membrane with a molecular weight cutoff of 12-14 kDa. The dialyzed solution was then filtered using a 0.20 μm vacuum filtration system and stored at −80° C. for at least 48 h. The frozen solution underwent lyophilization in a Labconco FreeZone 4.5L Benchtop freeze dryer (Labconco, MO, USA) at vacuum pressure ˜0.016 mbar until a solid GelMA product was obtained. The degree of methacryloyl substitution for three independently synthesized batches used in this study was 75±1%, 73±2%, and 75±1% (n=3, for each batch), determined according to published protocol.

### Methods—Microgel Fabrication

GelMA solutions (7 or 14% w/v) were prepared by dissolving the lyophilized GelMA polymer in DPBS, containing LAP photoinitiator (0.1% w/v). Additionally, PEG solutions with varying concentrations (3, 4, or 5% w/v) were prepared by dissolving the PEG polymer in DPBS, containing LAP (0.1% w/v). For the GelMA-PEG mixtures, the final precursor polymer solution was created by mixing equal volumes of the GelMA (14% w/v) and PEG solutions. The GelMA (7% w/v)-PEG mixtures or a GelMA solution (7% w/v) formed the dispersed phase, while the continuous phase comprised Novec™ 7500 Engineered Fluid, supplemented with Pico-Surf™ (0.5% v/v). The two phases were introduced into a step emulsification microfluidic device, fabricated based on a published work (adopted from an established protocol) via two syringe pumps (PHD 2000, Harvard Apparatus, MA, USA). The microfluidic droplet fabrication setup was maintained at 37-40° C. using a space heater to prevent GelMA physical gel formation during the droplet fabrication. The resulting droplets were collected in microcentrifuge tubes, placed in water baths at fixed temperatures to induce phase separation. The bath temperature was monitored using a digital J/K type thermometer (TM100, Extech Instruments, NH, USA). Subsequently, the droplet suspensions were stored at 4° C. overnight, leading to the formation of physically crosslinked microgels.

### Methods—Measuring the Cooling Rate of Droplet Suspensions

To evaluate the average cooling rate, the droplet suspension in oil, stored in a 1.7 mL microcentrifuge tube, was initially placed in a water bath at 37° C., marked as the initial temperature (Tᵢ). The droplet suspension was then transferred to another water bath at a fixed temperature (0, 10, or 20° C.), and the suspension temperature was recorded every 5 s until the final suspension temperature (Tf) reached the water bath temperature (0, 10, or 20° C.) and remained constant for 15-20 s.

### Methods—Measuring Phase Separation Temperature

To determine the phase separation temperature of varying GelMA-PEG mixtures, polymer solution mixtures containing a fixed GelMA concentration (7% w/v) and varying PEG concentrations (1.5, 2, or 2.5% w/v) were pipetted into pre-warmed (37° C.), laser-cut, disk-shaped reservoirs (diameter=10 mm and height=1 mm) and sandwiched between two microscope slides. The samples were then placed in a temperature-controlled bold line stage top incubator (Okolab, Pozzuoli, Italy) set to 37° C. The temperature was gradually reduced from 37° C. to 25° C. at 1° C. intervals every 30 min. For temperatures below 25° C., the reservoirs were transferred onto a temperature-controlled hotplate, and the temperature was decreased from 25° C. to 21° C. in 1° C. intervals every 30 min. Throughout this process, at each interval, the samples were closely monitored and imaged using a DMi8 THUNDER Imager three-dimensional (3D) Cell Culture microscope (Leica Microsystems, Germany). The phase separation temperature was reported as the average of temperatures at which phase-separated patterns in microgels were observable using microscopy images in three independently prepared samples.

### Methods—GHS Formation

The physically crosslinked microgels, phase-separated at varying T_{f} (0 or 20° C.), were washed once with PFO (20% v/v in Novec™ 7500 Engineered Fluid) at ˜4° C., using a 1:1 volume ratio, to remove the surfactant and oil from the suspension. The suspension was then mixed with 200 μL of a LAP solution (0.1% w/v in DPBS), followed by vortexing, centrifugation at 325×g for 15 s, and supernatant removal. This process was repeated twice. The microgels were then vigorously vortexed for 15 s and allowed to sediment after which the supernatant was collected and replaced with a fresh LAP solution (0.1% w/v in DPBS). This process was repeated three times. To maintain the microgels physically crosslinked during the washing steps, a cold-water bath (temperature ˜4° C.) was used. The washed microgel suspension was then centrifuged at 2940×g for 15 s, and the supernatant was removed. The packed microgel suspension was pipetted into laser-cut cylindrical molds with varying dimensions using a positive displacement pipette (Microman E M100E, Gilson, OH, USA). Finally, the molded microgels underwent photocrosslinking via light (UV LED Flood Light, source power=20 W, wavelength=395-405 nm, QUANS, China) exposure for 2 min at an intensity of ˜15 mW cm⁻².

### Methods—Microgel Pore Characterization

The aqueous microgel suspension was centrifuged at 325×g for 15 s, and the supernatant was removed. Subsequently, 100 μL of microgels were mixed with 900 μL of DPBS, containing LAP (0.1% w/v), and exposed to the light (395-405 nm and 15 mW cm⁻²) for 2 min while stirring at 1000 rpm in 2 mL microcentrifuge tube on a magnetic stirrer (Four E's Scientific, China) at 4° C. The individually photocrosslinked microgels were then incubated in 200 μL of FITC-dextran solution (12 μM in DPBS) for 30 min. Z-stacks of FITC-incubated microgels (average height ˜80 μm, ˜160 slices, height increment size ˜0.5 μm) were imaged from the surface to the center of each microgel using a DMi8 THUNDER Imager 3D Cell Culture microscope, equipped with a 25× objective (HC FLUOTAR L 25×/0.95 W VISIR, Leica Microsystems, Germany). Then, 3D images were rendered from the Z-stacked images using the Leica application suite X (LAS X, version 3.7.4.23463). The void fraction and average median pore size of individual microgels were characterized using MATLAB (MATLAB, version R2023b), respectively. The void fraction was determined by summing the area of FITC-labeled pixels and dividing it by the area enclosed by detected edges, which were identified as the last local minimum in the FITC-occupied area before reaching the highest FITC-occupied area. Average median pore size of microgels was determined two-dimensionally (2D) by skeletonizing FITC-labeled areas and obtaining the median of distances from each point along the skeleton to the adjacent unlabeled region for each layer of the Z-stacks. The median pore size of layers was then averaged and reported for each microgel (n>16).

### Methods—GHS Pore Characterization

To assess the GHS void fraction and median pore size, 100 μL of a FITC-dextran solution (12 μM in DPBS) was added on top of the scaffolds (diameter=8 mm and height=0.4 mm), followed by incubation for 30 min at room temperature. The scaffolds were then imaged in 2D at the layer of microgel-microgel contact. GHS median pore size was characterized using MATLAB as described earlier in section 1.7. Void fraction was assessed using a MATLAB code by calculating the ratio of FITC-labeled area to the entire area within the randomly selected regions of interests (ROIs) (n=5 samples). Measuring void fractions using 3D images constructed from Z-stacks was not feasible with the DMi8 THUNDER Imager 3D Cell Culture microscope or the Leica DMi8 laser scanning confocal microscope equipped with STELLARIS 5 White Light Lasers (Leica Microsystems, Germany). The imaging was limited to a certain depth from the glass surface to approximately the center of the microgels, where the highest microgel-microgel contact was observed. Imaging beyond this depth proved impossible, likely because of the porous structure of the microgels, which caused significant light refraction. For void fraction analysis, 2D images were acquired at the depth where the highest microgel-microgel contact was observed, which provided the lowest void fraction in the scaffold. Additionally, the void fraction observed in samples made with nonporous microgels at this depth was consistent with previous observations, where the void fraction of GHS made with microgels of similar size (˜170 μm) was reported to be 20-25%. Median pore size was determined by skeletonizing the FITC-labeled areas in images, wherein maximum microgel-microgel contact was observed, and obtaining the median of distances from each point along the skeleton to the adjacent unlabeled region (n=5 samples).

### Methods—Mechanical Characterization of GHS

To measure the compressive modulus of GHS, cylindrical specimens (diameter=8 mm and height=3 mm) were fabricated and incubated in DPBS at room temperature overnight. The samples were then subjected to a compression test using an Instron mechanical tester (Instron 5943, MA, USA) at a compression rate of 1 mm min⁻¹. The slope of elastic region in the compressive stress-strain curve (within 0.05-0.15 mm mm⁻¹ strain) was measured and reported as the compressive modulus of GHS.

### Methods—Rheological Characterization of GHS

Oscillatory rheological tests were performed on disk-shaped specimens (diameter=8 mm and height=1 mm) using an AR-G2 rheometer (TA instrument, DE, USA), equipped with 8 mm diameter top and 25 mm bottom sandblasted plates. All the fabricated samples were incubated in DPBS at room temperature overnight, followed by conducting the tests at 25° C. The scaffolds remained hydrated during the test via adding a few DPBS droplets around them. The storage and loss moduli were determined through a frequency sweep test, conducted at constant strain ˜0.1% within the linear viscoelastic region (LVR) and frequency ˜0.1-100 rad s⁻¹. The LVR was identified by an amplitude sweep test at strain ˜0.01 to 1000% and a fixed frequency (1 rad s⁻¹).

### Methods—Scanning Electron Microscopy (SEM) Imaging

Ethanol solutions of varying concentrations (30, 50, 60, 70, 80, 90, and 95% v/v) were prepared using ultrapure water. Disk-shaped GHS specimens (diameter=8 mm and height=3 mm) were placed in a Petri dish, containing 10 mL of 30% v/v ethanol, and incubated at room temperature for 10 min. The samples were then sequentially incubated in Petri dishes with increasing ethanol concentrations at 10-min intervals. After the final incubation in a 95% v/v ethanol solution, the specimens were incubated in 10 mL of 100% ethanol for a 30 min at room temperature. The last step was repeated two more times. Following the final ethanol incubation, the scaffolds underwent supercritical drying using a critical point dryer (CPD300, Leica Microsystems, Germany). To prepare the samples for SEM imaging, they were coated with iridium (thickness˜5.6 nm) using a low vacuum sputter coater (Leica EM ACE200, Germany). The surface properties of the GHS specimens, after undergoing the supercritical drying process, were visualized using a scanning electron microscope (Quanta 250 ESEM, Thermo Scientific, OR, USA) at an accelerating voltage of 3-5 kV and a beam current of 0.67 nA for scaffolds made with nonporous microgels and 53 pA for scaffolds made with porous microgels.

### Methods—In Vitro Cell Culture

The NIH/3T3 murine fibroblast cells (ATCC, VA, USA) were cultured in DMEM, containing 10% v/v FBS and 1% v/v antibiotics. The culture was maintained in a cell culture incubator (Eppendorf, Hamburg, Germany) under a 5% v/v carbon dioxide (CO₂) atmosphere at 37° C. Cells were passaged once they reached ˜80% confluency by detaching them from T-75 cell culture flasks using a trypsin-EDTA solution (0.25%) and counted using a cell counter (Countess™ II automated cell counter, Thermo Fisher Scientific, MA, USA). To conduct in vitro studies involving GHS, cells were mixed with microgels that were packed at 2940×g for 15 s to obtain a cell density of 4×10³ per μL of packed microgel suspension. Subsequently, cell-microgel mixture was pipetted into cylindrical molds (diameter=6 mm and height=3 mm) using a positive displacement pipette and photocrosslinked via light exposure (395-405 nm and 15 mW cm⁻²) for 2 min.

### Methods—Assessing Cell Infiltration into Porous Microgels

To evaluate cell infiltration within individual porous or nonporous microgels, the aqueous microgel suspensions were centrifuged at 325×g for 15 s, followed by supernatant removal. Subsequently, the microgels were mixed at a 1:10 ratio with a LAP solution (0.1% w/v in DPBS) and individually photocrosslinked using light (395-405 nm and 15 mW cm⁻²) for 2 min at 4° C., while being stirred at 1000 rpm to prevent interparticle crosslinking. Photocrosslinked microgels were then centrifuged at 325×g for 15 s, followed by supernatant removal. Afterwards, 200 μL of individually crosslinked, packed microgels were added to a non-treated Petri dish, containing 5 mL of cell suspension in culture media (10⁶ NIH/3T3 murine fibroblast cells per mL). The suspension was then thoroughly mixed via pipetting and maintained under a 5% CO₂ atmosphere at 37° C. in the cell culture incubator. After 48 h, cells were fluorescently labeled through the incubation of cell-microgel suspension in 5 mL of calcein AM solution (1 μM in 10 mL of live cell imaging solution) at room temperature for 30 min. The suspension was then vortexed for 10-15 s, using a Fisherbrand™ variable speed mini vortex mixer (Thermo Fisher Scientific, MA, USA), to disintegrate microgel-cell aggregates, resulting in individual cell-adhered microgels. Imaging was conducted using the DMi8 THUNDER Imager 3D Cell Culture microscope at excitation/emission wavelengths of 470 nm/510 nm to obtain the Z-stacks of single microgels with an average Z-depth of ˜90 μm and a height increment size of 0.5 μm. All the Z-stacks were analyzed in each layer (2D) to obtain the total cell-infiltrated volume in each microgel using MATLAB. Briefly, images were blurred using a Gaussian noise deconvolution and binarized using adaptive thresholding. Afterwards, edges were detected, and the background was masked. The labeled pixels within the unmasked area were summed to find the area occupied by cells for each layer. This area was then multiplied by Z-step size, and the process was repeated over the entire Z-stack for each layer. The reported volume of infiltrated cells was calculated by summing the volume of all layers.

### Methods—Cell Viability Assessment

The live/dead cell viability assay was used to assess the viability of cells cultured in the GHS, fabricated using porous or nonporous microgels. To this end, NIH/3T3 murine fibroblast cells were mixed with microgels to obtain a cell density of 4×10³ per μL of microgels (packed via centrifugation at 2940×g for 15 s). Then, the mixture was pipetted into cylindrical molds (diameter=6 mm and height=3 mm) and photocrosslinked using the light (395-405 nm and 15 mW cm⁻²) for 2 min. A two-color fluorescence approach was implemented using calcein AM for live cell staining and BOBO-3 iodide for dead cell detection. Briefly, 1 mL of calcein AM (1 μM) was added to 1 μL of BOBO-3 iodide and mixed to prepare a stock solution. The stock solution was diluted by adding 1 mL of live cell imaging solution to prepare the staining solution. Subsequently, 500 μL of staining solution was added to each cell-laden scaffold, and samples were incubated for 30 min at room temperature. Following the incubation, samples were imaged using the DMi8 THUNDER Imager 3D Cell Culture microscope. The live cell channel was set to excitation/emission wavelengths of 470 nm/510 nm, and the dead cell channel was set to excitation/emission wavelengths of 550 nm/610 nm. The live cell heatmaps in GHS samples were generated by converting the images to 8-bit format and adjusting their size to 50 pixels×50 pixels using FIJI ImageJ software (version 1.54f, NIH, MD, USA). To stain cells with Alexa Fluor™ 488 phalloidin and DAPI, samples were first fixed in a PFA solution (4% v/v in ultrapure water) for 45 min. Scaffolds were then incubated in DPBS for 15 min at room temperature, three times, followed by DPBS removal. Next, the samples were permeabilized by incubating in a Triton X-100 solution (0.3% v/v in DPBS) for 10 min, followed by incubation in DPBS for 10 min at room temperature, repeated three times. After DPBS removal, samples were incubated in the Alexa Fluor 488 phalloidin solution (1:400 volume ratio in DPBS) for 60 min at room temperature. Afterward, they were incubated in the live cell imaging solution for 15 min three times. Following this step and for nucleus staining, samples were incubated with a DAPI solution (1:1000 volume ratio in DPBS) for 5 min at room temperature, and then incubated in the live cell imaging solution, for 10 min, two times. Finally, the samples were imaged using the DMi8 THUNDER Imager 3D Cell Culture microscope, equipped with a 25× water contact objective (HC FLUOTAR L 25×/0.95 W VISIR). Nucleus circularity for cells infiltrated into inter-or intraparticle pores in GHS made with porous or nonporous microgels was measured using the maximum intensity projection (MIP) of 3D Z-stacked images. Images were analyzed using FIJI ImageJ software, where they were converted to the 8-bit type, processed using the watershed function, and assessed for nucleus circularity. For this analysis, at least 100 cell nuclei were examined in each scaffold, and three scaffolds were analyzed for each study group.

### Methods—Metabolic Activity Assessment

The PrestoBlue HS cell viability kit was used to assess the cell metabolic activity throughout the cell culture period. Briefly, PrestoBlue was mixed with serum-free DMEM at a 1:10 volume ratio, and 1 mL of the final solution was added to each well of a 24-well plate. Each well had a scaffold (diameter=6 mm and height=3 mm) fabricated using a cell-microgel mixture, containing 4×10³ cells per μL of microgels (packed via centrifugation at 2940×g for 15 s), as mentioned in section 1.11. After incubation for 3 h at a 5% CO₂ atmosphere and 37° C., 100 μL of each supernatant was pipetted into a well of a 96-well microplate, and the fluorescence intensity was measured using a microplate reader (Tecan Infinite M Plex, Männedorf, Switzerland) at the excitation/emission wavelengths of 530 nm/590 nm. The resulting fluorescence intensity was adjusted with respect to the background signal obtained from the virgin PrestoBlue/media solution incubated in a cell-free well in the 24-well plate for 3 h under a 5% CO₂ atmosphere at 37° C.

### Methods—In Vivo Subcutaneous Implantation Mouse Model

Animal experiments were carried out following the protocol (#02132) approved by The Pennsylvania State University Institutional Animal Care and Use Committee (IACUC). For subcutaneous implantation, based on a power analysis (see Statistical Analysis section), 12 C57BL/6 mice (age=10 weeks, 6 male and 6 female, The Jackson Laboratory, CT, USA) were used. Prior to the surgery, mice were acclimated for 8 days. For the surgery, mice were anesthetized via inhalation of 2% isoflurane carried in oxygen at 5 L min⁻¹. Meloxicam (0.5 mL kg-1) was administered subcutaneously to the anesthetized mice. The dorsal skin was shaved, and surgical site was prepped with ethanol (70% v/v) and chlorhexidine® scrub (2% v/v). Two pockets were formed on each mouse, and a cuboid scaffold (length=7 mm, width=7 mm, height=3 mm) from each study group was implanted subcutaneously in each pocket. In total, 8 samples for each study group were evenly distributed between male and female mice and randomized to ensure no mice received implants from the same group. Following the surgery, each mouse was placed in an individual cage and housed in a standard day/night light cycle environment, provided with access to food and water. All the mice were checked daily, and their weights were recorded. Mice were euthanized using CO₂ (3 L min⁻¹) 2 weeks after scaffold implantation, and samples along with the surrounding tissues were collected and stored in a 4% v/v PFA solution overnight. Samples were then transferred to an ethanol solution (70% v/v in DPBS).

### Methods—Hematoxylin and Eosin (H&E) and Immunofluorescence Staining

Fixed samples were transferred into cassettes and processed using a Leica TP1020 Automatic Benchtop tissue processor (Leica Biosystems, Germany) with ethanol gradient and xylene immersions, followed by paraffin embedding. To this end, samples were first immersed in 70% v/v ethanol for 30 min, followed by immersion in 85% v/v ethanol for 45 min, and then in 95% v/v ethanol for 40 min (twice). Next, the samples were immersed in 100% v/v ethanol for 40 min (twice). Afterward, the samples were immersed in xylene three times, each for 40 min, and finally immersed in paraffin twice, each time for 45 min. Paraffin-embedded samples were then sectioned using a Shandon™ Finesse™ paraffin microtome (Thermo Fisher Scientific, MA, USA) (thickness ˜7-10 μm) and affixed onto Diamond® white glass microscope slides.

For H&E staining, sections were loaded onto a Leica Autostainer ST5010 XL (Germany) and deparaffinized by heating in an oven at 58° C. for 18 min, followed by three times immersion in xylene, each lasting for 150 s. The sections were then immersed in a descending ethanol gradient, first in 100% v/v and then in 95% v/v, each for 90 s, followed by immersion in tap water for 1 min. Samples were then immersed in staining reagents, including Hematoxylin 560™, Alcoholic Eosin Y 515™, Blue Buffer 8™, and Define®. This was followed by immersion in an ascending ethanol gradient, first in 95% v/v and then in 100% v/v, each for 90 s, and finally in xylene for 4 min.

For immunofluorescence staining analyses, the sections were loaded onto a Leica Autostainer ST5010 XL (Germany). The samples underwent deparaffinization by heating in an oven at 58° C. for 18 min, followed by three immersions in xylene, each lasting 10 min. The sections were then immersed in a descending ethanol gradient. This involved two immersions in 100% v/v ethanol for 3 min each, followed by immersions in 95% and 85% v/v ethanol for 2 min each, and a final immersion in 70% v/v ethanol for 3 min. Dewaxed sections were then washed by two times incubation in tap water for 4 min. Samples were then immersed in PBS for 5 min. Subsequently, the dewaxed samples were subjected to heat-mediated antigen retrieval overnight in a 10 mM citrate buffer (pH=6.0) at 60° C. The sections were blocked with BSA (1% w/v) and TWEEN® 20 (0.3% v/v) in PBS for 1 h at room temperature, which were then stained against α-SMA, CD31, and CD68. To this end, the sections were incubated with recombinant Alexa Fluor® 488 anti-CD31 antibody (1:100), anti-α-SMA (1:100), and Alexa Fluor® 647 anti-CD68 antibody (1:100), each prepared in a 1% w/v BSA solution in PBS, overnight at 4° C. After rinsing with PBS, samples were mounted, counterstained using Fluoroshield™ with DAPI, and imaged using the DMi8 THUNDER Imager 3D Cell Culture microscope. Image analysis involved converting images to an 8-bit type and thresholding using FIJI ImageJ software. Multiple rectangular ROIs (width=400 μm and length=800 μm) were selected for the image analysis. Cell density (%) was measured as the fraction of DAPI fluorescence area over the total area of ROI. Cell infiltration at varying depths was quantified by measuring cell density in 4adjacent rectangular ROIs (width=200 μm and length=400 μm), starting from the tissue-scaffold interface. This value was then normalized with the summation of cell density across all 4 ROIs. The CD31-, CD68-, and α-SMA-stained area were also measured as the fraction of fluorescence area over the total area of each ROI (width=400 μm and length=800 μm). All the ROIs were selected from the tissue-scaffold interface. The in vivo cell nucleus heatmaps in subcutaneously implanted GHS were generated by converting images to 8-bit type and adjusting their size to 50 pixels×50 pixels using FIJI ImageJ software.

### Methods—Statistical Analysis

G*Power software (version 3.1.9.6) was used to calculate the number of mice (n=8) based on a minimum 80% power, type I error of 0.05, and the effect size of 1.4. Some scaffolds were unable to be analyzed, leading to sample sizes of n=7 for the GHS comprising nonporous microgels or large-pore microgels, and n=6 for the GHS made up of small-pore microgels. The effect size for the n=6 condition was 1.6 (GHS-S) and 1.5 for the n=7 conditions (GHS-L and GHS-N) to maintain 80% power. Sections from all explanted scaffolds were analyzed for cell infiltration and immunofluorescence staining. Statistical significance was determined using unpaired two-tailed t-test or one-way/two-way analysis of variance (ANOVA), followed by Tukey's post-hoc multiple comparison test between the study groups. All the data reported in this study were acquired with at least three iterations (n≥3). The statistical analysis was performed using GraphPad Prism for Windows (version 10.4.0, MA, USA). The level of significance was noted with ns: non-significant p≥0.05, *p<0.05, **p<0.01, ***p<0.001, and ****p <0.0001.

### Results and Discussion

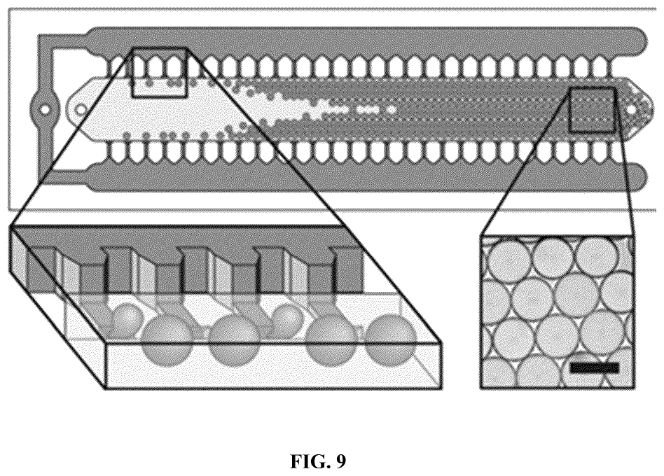

To fabricate porous microgels as the building block of GHS, a homogenous precursor solution, consisting of GelMA, PEG, and LAP is injected into a high-throughput step emulsification microfluidic device. FIG. 9 shows the formation of near-monodispersed droplets suspended in a continuous oil phase, wherein droplets are stabilized using a surfactant. GelMA, a photocrosslinkable protein-based biopolymer, has widely been used to fabricate biocompatible and biodegradable hydrogel biomaterials, containing bioactive peptide sequences that enable cell attachment and matrix metalloproteinase (MMP)-mediated degradation. Similarly, PEG has been selected as a key synthetic polymer for biomedical applications given its tunable physicochemical properties and inertness.

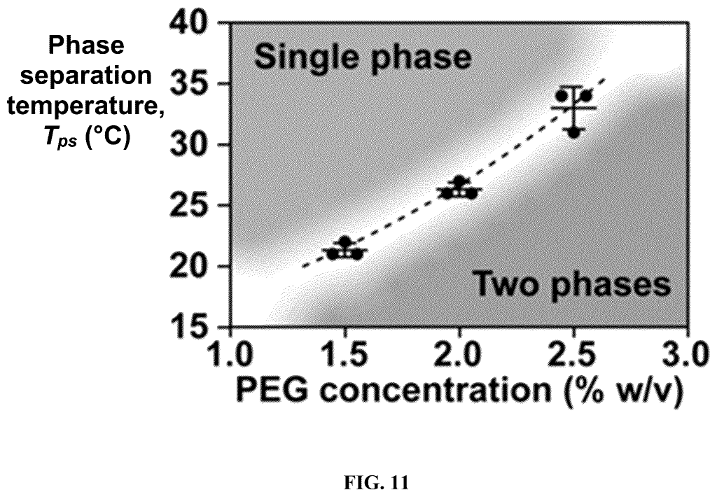

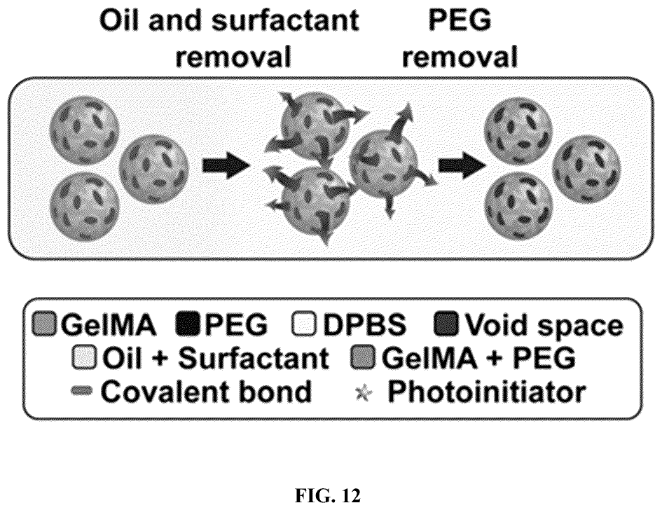

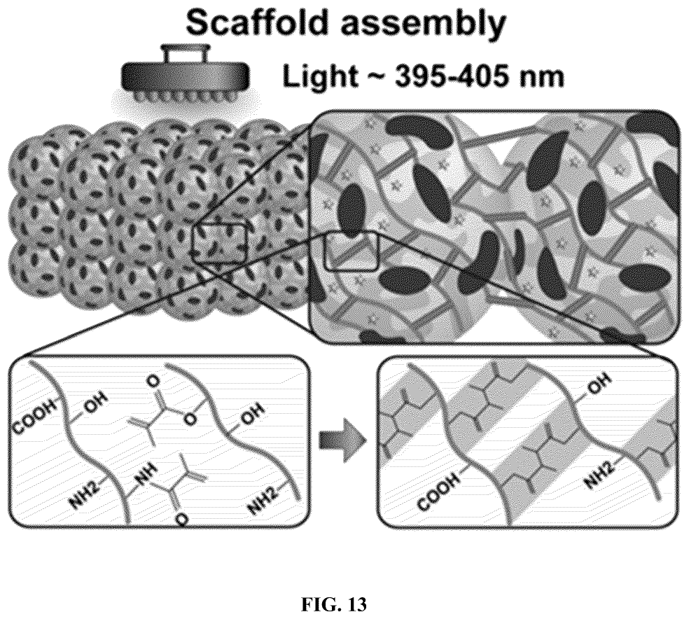

The homogeneous solution of GelMA and PEG shows partitioning behavior, which may be controlled by adjusting the composition or temperature of mixture. Here, the homogenous single-phase droplets containing GelMA-PEG polymers at Tᵢ (initial temperature) are thermally phase separated by lowering the temperature to T_{f} (final temperature). This process is attributed to the increase in the strength of intermolecular hydrogen bonding between gelatin chains upon the addition of PE. As a result, random-coil gelatin chains undergo a partial reversion to triple-helical structures, inherited from collagen. This alteration subsequently reduces the miscibility of gelatin with PEG, as illustrated in FIG. 10. FIG. 11 shows the effect of PEG concentration on the phase separation temperature (T_{PS}) of binary GelMA (7% w/v)-PEG mixture. By cooling homogenous droplets to a temperature below the phase transition temperature, the homogeneous GelMA-PEG solution in droplets phase separates into two distinct phases, which may be halted by GelMA physical gel formation. The phase-separated microgels are then stored at 4° C., and the oil is then removed from the microgel suspension after which PEG polymer chains diffuse out of the microgels, yielding micron-sized pores within individual microgels, as shown in FIG. 12. FIG. 13 presents the formation of GHS, featuring hierarchical interconnected pores at both inter-and intraparticle scales via porous microgel jamming and subsequent chemical crosslinking through light exposure. The formation of covalent bonds via the free radical polymerization of GelMA's methacryloyl groups accounts for both intra- and interparticle crosslinking, yielding stable constructs, as reported for the GHS made up of nonporous microgels.

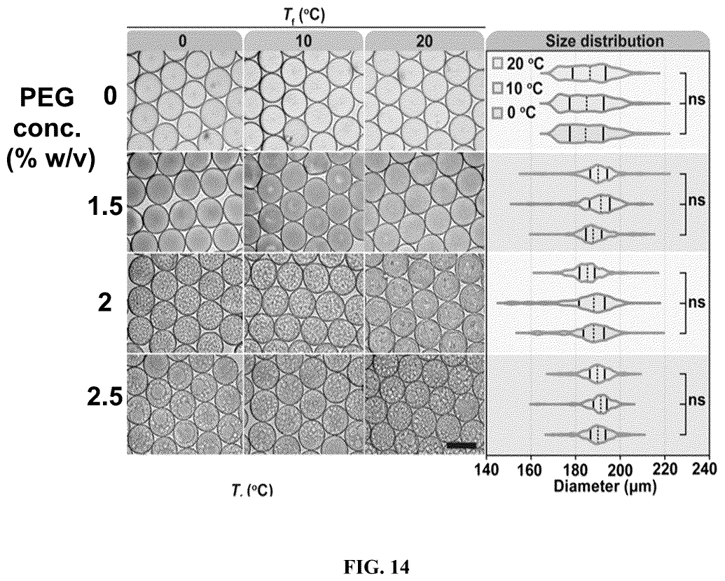

The individual porous microgels are first characterized, followed by GHS formation and characterization. FIGS. 14-17 present the size and pore characterizations of individual microgels after undergoing thermally-induced phase separation. The precursor polymer solution, prepared via mixing PEG (initial concentration=0, 3, 4, or 5% w/v) and GelMA (initial concentration=14% w/v) solutions at a 1:1 volume ratio, is introduced into a high-throughput step emulsification microfluidic device as a dispersed phase, resulting in the formation of droplets in a continuous oil phase. Droplets are then collected in a water bath at 37° C. (Tᵢ) to remain as a homogenous GelMA-PEG mixture. To induce phase separation for fabricating porous microgels, droplets are immersed in an aqueous bath at a fixed T_{f} (0, 10, or 20° C.), which is monitored using a thermometer. The applied temperature gradient reduces temperature at varying cooling rates. As the GelMA-PEG mixture is cooled below T_{PS}, the thermodynamic incompatibility between GelMA and PEG leads to liquid-liquid phase separation.

As the size of droplets and subsequent microgels produced by the step emulsification device is controlled by the channel height (˜60 μm), we anticipate a consistent and narrow size distribution for the phase-separated microgels despite varying final (water bath) temperatures. FIG. 14 shows the bright-field microscopy images of phase-separated microgels at varying T_{f} and PEG concentrations, along with their corresponding size distribution. A narrow size distribution with an average diameter of 187±10 μm across all PEG concentrations (0, 1.5, 2, 2.5% w/v) and T_{f} (0, 10, or 20° C.) (n>5000 over all conditions) is obtained. Also, the particle size analysis at each PEG concentration (n>300) shows no significant difference among microgel groups prepared at varying T_{f}.

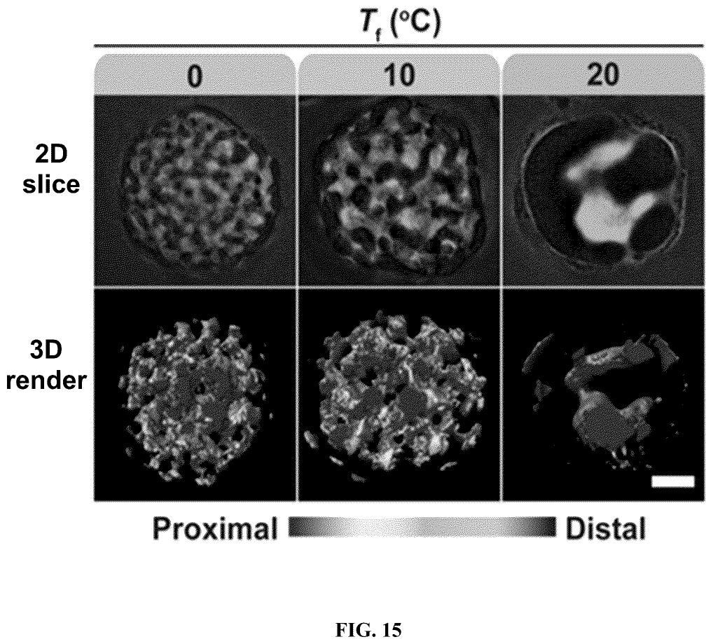

To characterize the porous structure of physically and chemically crosslinked individual microgels and to investigate the effect of cooling rate on phase separation, a high-molecular weight fluorescent dextran (average molecular weight=2 MDa) is used, occupying the intraparticle void spaces. FIG. 15 presents 2D slices near the center of fluorescently labeled microgels (initially contained 2% w/v PEG), as well as their 3D renderings wherein the 3D-constructed images show interconnected pores inside individual microgels. Importantly, the cooling rate of microgel suspension during the immersion step influences the phase separation process and, consequently, the resulting microgel pore structure. The data are averaged and fitted using a single-phase decay formula (n=3): T(t)=(Tᵢ−T_{∞})e^{λt}+T_{∞}, where T(t) is the microgel suspension temperature at any given time, Tᵢ is the initial microgel suspension temperature, λ is the decay constant, t is the time elapsed, and T_{∞} is the T value at infinite time, which is close to T_{f}. At T_{f}=0, 10, or 20° C., the non-linear fit to the data results in λ₀=0.07434 s⁻¹ with R²=0.997, λ₁₀=0.04425 s⁻¹ with R²=0.979, or λ₂₀=0.03808 s⁻¹ with R²=0.995, respectively. The subscript of 2 indicates the T_{f} for each condition. As T_{f} decreases from 20° C. to 0° C., the cooling rate or decay constant increases significantly, which causes the GelMA-PEG mixture to pass through T_{PS} more quickly and reach the GelMA physical gel formation temperature faster, thereby halting phase separation and yielding unique phase separated patterns. Previous studies have demonstrated that in the absence of gel formation, phase separation patterns evolve over time as each phase self-organizes to minimize the total interfacial energy. When the components in the aqueous phase undergo both phase separation and rapid gel formation (e.g., GelMA), they lack the time required to fully reach an energy-minimized configuration, leading to incomplete phase separation. Consequently, the lowest decay constant produces patterns closer to the final uninterrupted phase-separated state, while the highest decay constant results in patterns resembling the initial states of phase separation.

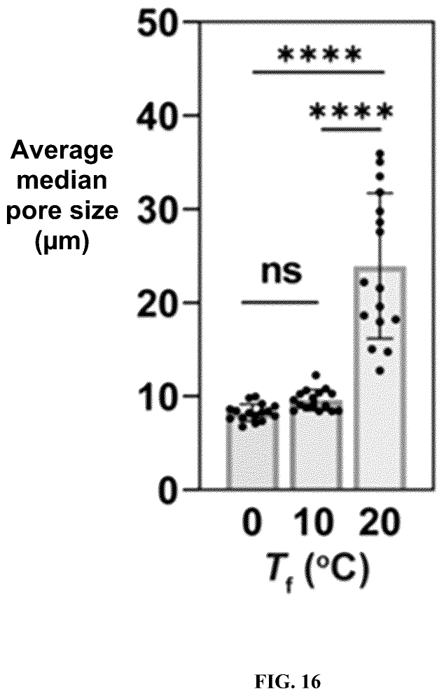

The porous architecture of individual microgels was further analyzed using a MATLAB code. FIG. 16 shows the average median pore size of porous microgels. Microgels that undergo phase separation at the lowest decay constant (i.e., 220=0.03808 s⁻¹) have a considerably higher average median pore size (˜24±8 μm) than their counterparts subjected to phase separation at 0° C. or 10° C., which had an average median pore size of ˜8±1 or ˜9±1 μm, respectively. Moreover, there is no significant difference in the average median pore size of the individual microgels phase separated at 0° C. and 10° C. When microgels are subjected to a higher cooling rate, they reach the gel formation temperature more rapidly, impeding complete phase separation, which leads to the formation of smaller pores.

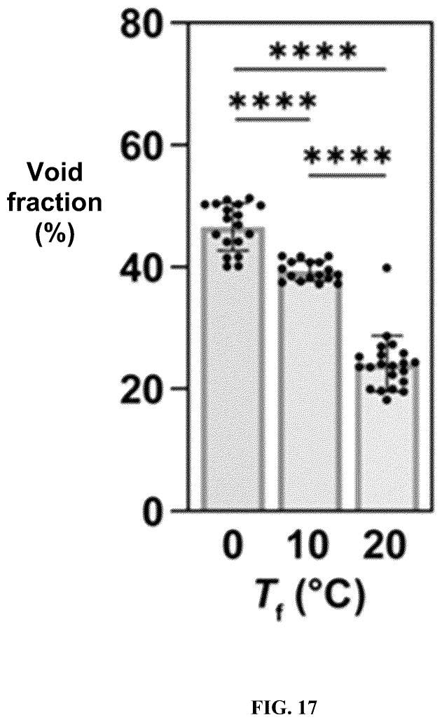

FIG. 17 presents the void fraction of porous microgels. The void fraction, defined as the ratio of fluorescently labeled void space to total microgel volume, is significantly affected by the cooling rate. The void fraction of microgels prepared at the cooling rate with the highest decay constant (i.e., λ_{0=0.07434} s⁻¹) is ˜46±4%. As the decay constant decreases by increasing T_{f}, the void fraction monotonically decreases from ˜39±2% (λ₁₀=0.04425 s⁻¹) to ˜24±5% at the lowest decay constant (λ₂₀=0.03808 s⁻¹). By reducing the final temperature, the heat transfer rate between the microgel suspension and surrounding environment increases. Consequently, when microgels are subjected to a lower cooling rate (e.g., T_{f}=20° C.), the slower heat transfer rate allows for more complete phase separation until the physical gel formation temperature is reached. The difference in the void fraction of microgels prepared at the lowest cooling rate (λ₂₀=0.03808 s⁻¹) and those prepared at the highest cooling rate (i.e., λ₀=0.07434 s⁻¹) is attributed to gel formation during phase separation. This process can lead to the formation of PEG-trapped states, which affect the interconnection of pores within the microgels, potentially leaving PEG residues inside and resulting in a lower void fraction in microgels with the lowest decay constant.

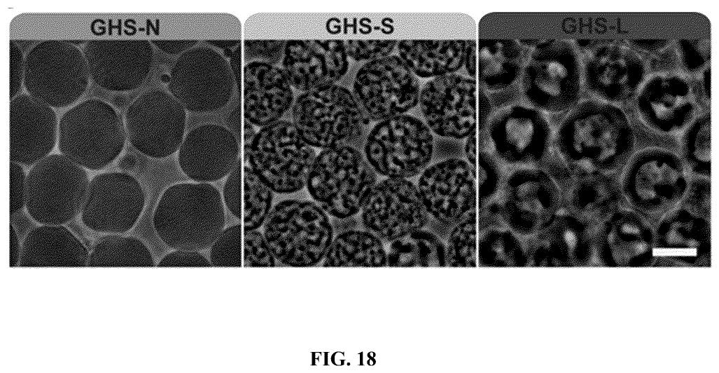

FIGS. 18-25 shows the pore and mechanical characteristics of GHS, fabricated from nonporous or porous microgels (initially contained 2% w/v PEG) that are phase-separated at 0° C. or 20° C. FIG. 18 presents intra-and inter-particle pores within the GHS made up of nonporous microgels (GHS-N), small-pore microgels (GHS-S), and large-pore microgels (GHS-L), visualized via a high-molecular weight fluorescent probe. While the fluorescent probe successfully penetrates the porous microgels, enabling the visualization of additional void spaces in GHS made up of porous microgels, nonporous microgels do not allow fluorescent molecule penetration. In conjunction with fluorescence microscopy, the structure of GHS is imaged using SEM. The results show that the non-porous microgels do not have any pores, and compared with the large-pore microgels, the small-pore counterparts have more pores on their surfaces. However, the pores on the surface of large-pore microgels are larger than those on the small-pore counterparts. These findings highlight the structural differences between the small- and large-pore microgels compared with the nonporous counterparts.

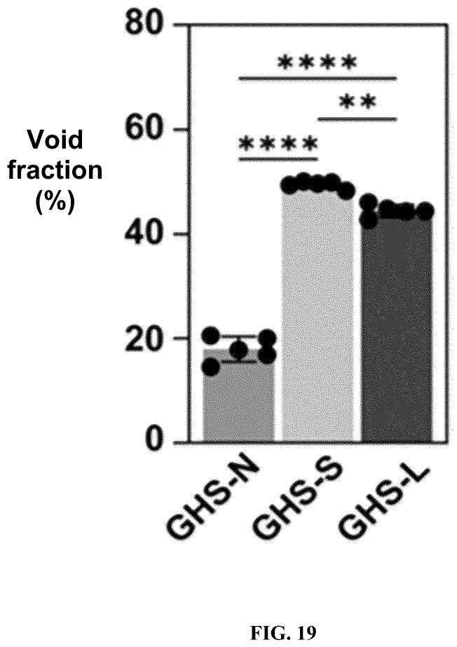

FIG. 19 shows the void fraction of GHS comprising nonporous or porous microgels. The micron-sized pores of scaffolds are quantified by analyzing the fluorescence images using a MATLAB code. The results show a significant difference between the void fraction of GHS-N, GHS-S, and GHS-L, wherein the GHS-S has the highest void fraction (49±1%), followed by the GHS-L (44±1%), and GHS-N (18±2%). The higher void fraction in GHS-S and GHS-L is attributed to the additional void spaces formed through the phase separation of GelMA-PEG mixture inside individual microgels, followed by PEG polymer removal. The theoretical porosity in scaffolds, φ_{theoretical}, can be calculated using the following formula: φ_{theoretical}(%)=φ_{GHS}(%)+φ_{μgel}(%)[100%−φ_{GHS}(%)], where φ_{GHS} is the average porosity (void fraction) of non-porous GHS (˜18%), and φ_{μgel} is the average porosity of individual microgels. The microgel porosity is multiplied by (100%-−φ_{GHS}) to account for the area expected to be occupied by microgels. When comparing the φ_{theoretical} with φ_{Observed} for each study group, a difference of 6-7% is observed, which is not surprising, given that φ_{observed} is measured experimentally. In contrast, when comparing φ_{theoretical}between GHS-S and GHS-L, an 18% difference is expected; however, the observed difference is ˜5%. This difference is attributed to underestimated porosity in GHS-S. At low-magnification images, some of the thinner pores in GHS-S do not produce a strong enough signal to be distinguished from random noise, causing them to remain unlabeled. As a result, the observed porosity is lower than the theoretical porosity calculated from the individual microgel porosity.

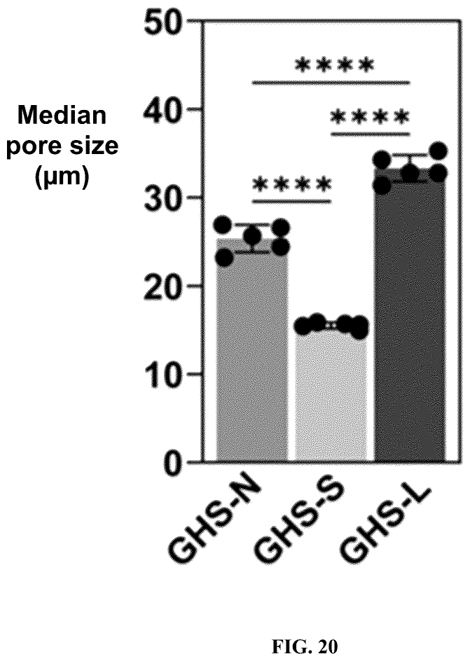

FIG. 20 presents the effect of microgel pore size on the average median pore size of GHS, where maximum microgel-microgel contact is observed. The average median pore size is 25±2 μm for GHS-N, 16±1 μm for GHS-S, and 33±2 μm for GHS-L. In comparison with the pore structure of GHS-N, GHS-S has a higher number of intraparticle pores with smaller sizes, yielding a lower average median pore size. Conversely, GHS-L has a higher number of large intraparticle pores than GHS-N, resulting in a higher average median pore size. Overall, these outcomes show that the hierarchical porous structure of GHS may be tailored via varying pore features of individual microgels, which is not trivial by using nonporous spherical microgels.

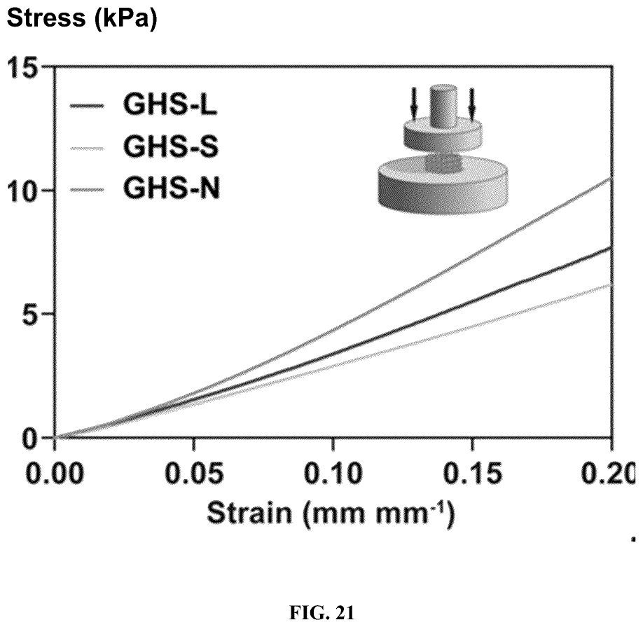

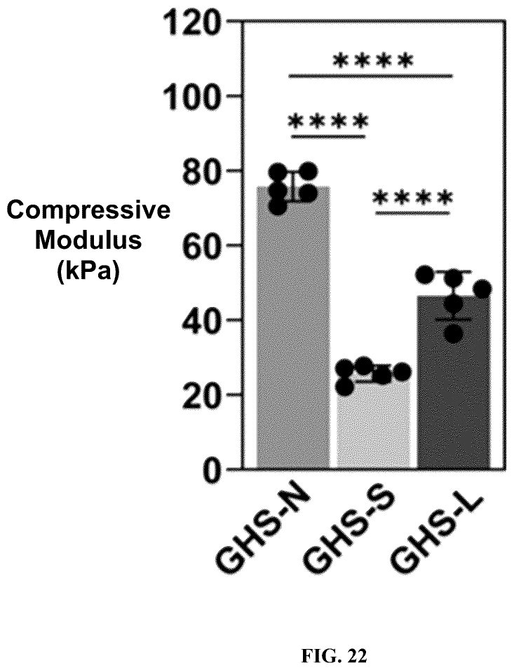

The mechanical and rheological properties of GHS, fabricated with nonporous or porous microgels, are evaluated using compression and oscillatory rheology tests, as presented in FIGS. 21-24. FIG. 21 shows the stress-strain curves, acquired via applying a compressive load to the scaffolds. In GHS-N and at any given strain, the stress is higher than that in GHS-S and GHS-L. FIG. 22 presents the compressive modulus of GHS, measured from the slope of compressive stress-strain curves in the linear elastic region, located at ˜5-15 mm mm⁻¹ compressive strain. The compressive modulus of scaffolds is inversely correlated with the void fraction: the scaffold with the highest void fraction, i.e., GHS-S, has the lowest modulus, and GHS-N has a significantly higher modulus than GHS-S and GHS-L.

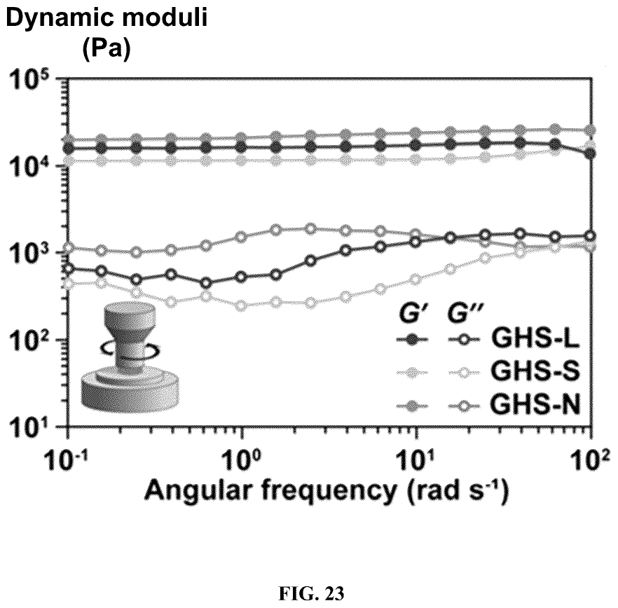

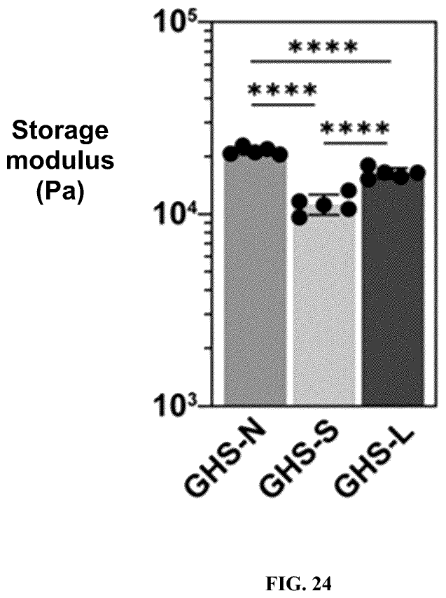

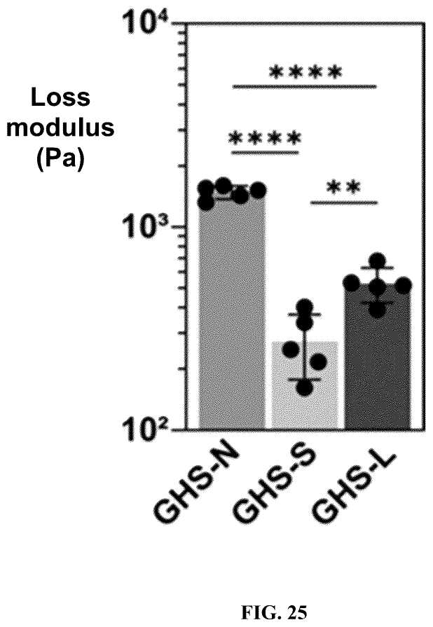

FIG. 23 shows the viscoelastic characteristics of scaffolds, evaluated via oscillatory rheology tests. The LVR is identified via an oscillatory strain sweep test at a constant frequency (1 rad s⁻¹). Then, storage (G′) and loss (G″) moduli are measured at a constant oscillatory strain of 0.1%, which is in the LVR, from 0.1 to 100 rad s⁻¹. Similar to the compression behavior reported earlier, the G′ of GHS-N is higher than that of GHS-S and GHS-L. FIG. 24 presents the average G′ of different study groups, measured at 0.1% strain and frequency of 1 rad s⁻¹. The average G′ of GHS-N is significantly higher than that of GHS-S or GHS-L, and GHS-L has a significantly higher G′ than GHS-S. FIG. 25, shows the average G″ of scaffolds at 0.1% oscillatory strain and frequency of 1 rad s⁻¹. A consistent trend akin to the G′ versus angular frequency is observed across all study groups for the G″. Additionally, in all study groups, G″ is at least an order of magnitude lower than the corresponding G′, which is a characteristic of gels.

The descending trend of compressive and storage moduli observed in the GHS, fabricated using the porous microgels compared with the counterpart made up of nonporous microgels may be attributed to the additional void spaces introduced by the porous microgels. Previous studies have shown that the GHS, fabricated with microgels of varying sizes, have significantly lower storage and compressive moduli compared with nonporous scaffolds. This difference has been attributed to the larger (macroscale) void spaces in GHS, which is absent in bulk hydrogel scaffolds. The larger void space in GHS results in lower interparticle contact area and consequently weaker interparticle contact among the microgels. A similar phenomenon may occur within individual microgels. In porous microgels, unlike their nonporous counterparts, the void spaces lead to a reduction in the mechanical strength. Such weakened mechanical strength at the microgel level affects the mechanical properties of the entire GHS.

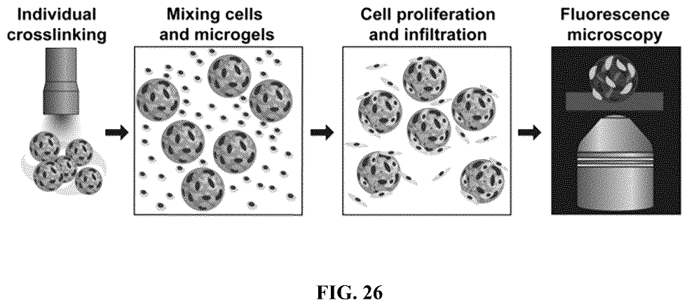

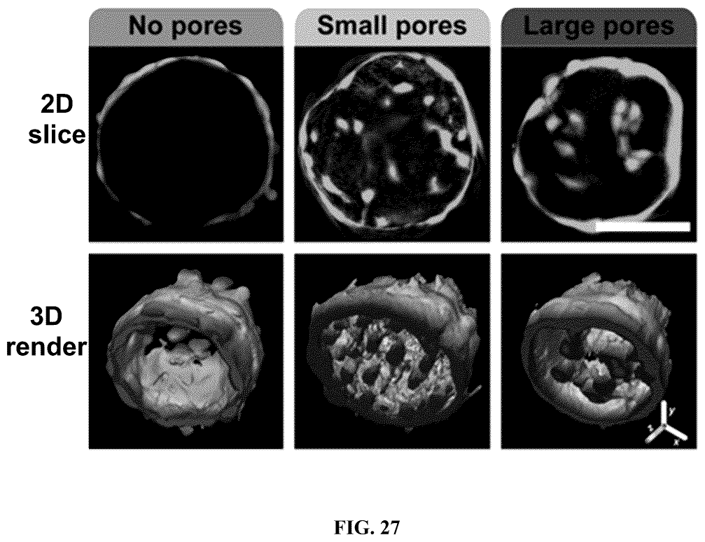

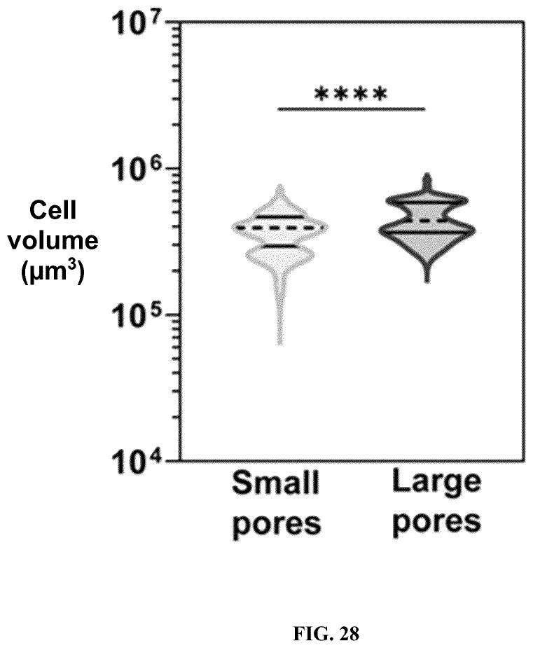

To evaluate the effect of microgel porous structure on the viability, proliferation, and migration of cells, in vitro studies are conducted using individual microgels, as well as the GHS, assembled from porous or nonporous microgels. FIG. 26 presents the schematic illustration of NIH/3T3 murine fibroblast cell interaction with individually photocrosslinked microgels. Phase-separated droplets are first physically crosslinked, followed by individual photocrosslinking. NIH/3T3 murine fibroblast cells are mixed with the microgels and cultured for 2 days. Then, cells are labeled with the calcein AM cell-permanent dye, and fluorescence images are acquired to study cell-microgel interactions, including adhesion and migration around/within microgels. FIG. 27 shows 2D cross-sectional and 3D-constructed images of microgels. Cells only adhere to the outer surface of nonporous microgels; however, they not only adhere to the porous microgel exterior, but also migrate into the intraparticle void spaces through the open pores. FIG. 28 presents the total volume of infiltrated cells into individual porous microgels. Cell volume, defined as the volume occupied by cells within the porous microgels, is calculated for small-and large-pore microgels. Accordingly, the cell volume in the large-pore microgels is significantly higher than that in the small-pore counterpart. These results are consistent with microgel pore characterization provided earlier: the median pore size of microgels that undergo phase separation with the lowest temperature decay constant (λ₂₀=0.03808 s⁻¹) are significantly higher than those prepared at the highest decay constant (λ₀=0.07434 s⁻¹), facilitating cell migration in the large-pore microgels compared with the small-pore counterparts.

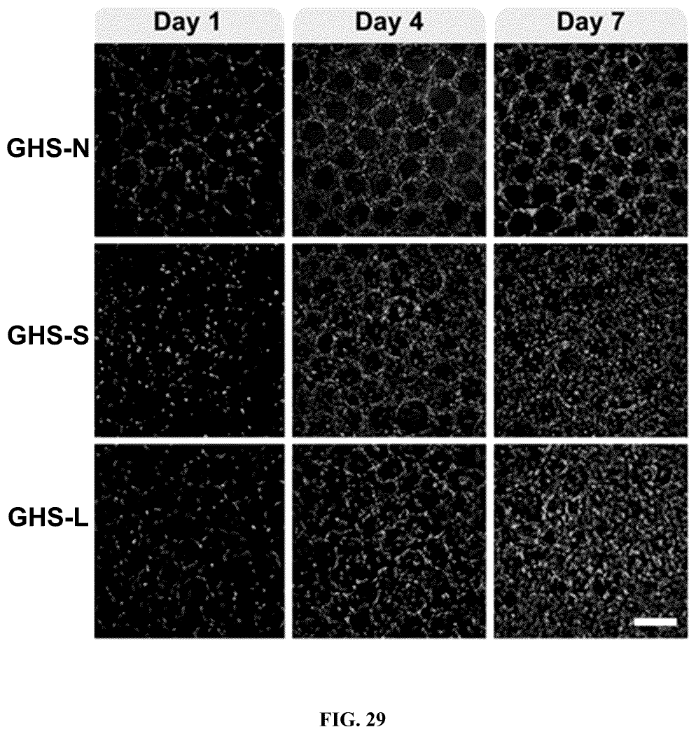

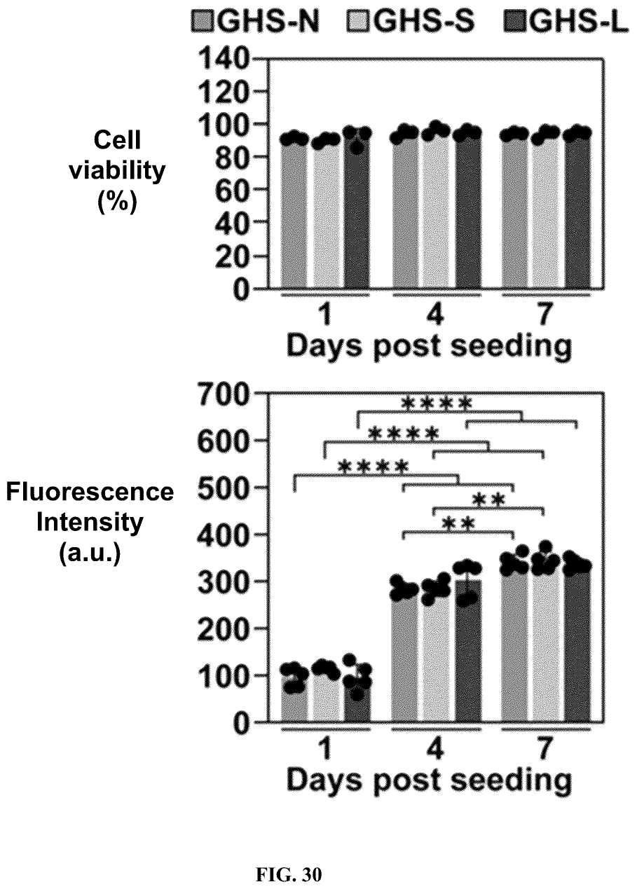

Cell viability, defined as the ratio of area occupied by live cells to the total area of live and dead cells, is characterized in GHS-N, GHS-S, and GHS-L. FIG. 29 shows the live/dead images of cell-laden scaffolds, fabricated using cell-microgel suspensions. Over the culture period, the number of live cells increases, as shown by the growing area of live cells adhering to the microgels and proliferating. Additionally, a low number of dead cells are observed in the scaffolds. Furthermore, in the GHS made up of porous microgels, a more uniform cell distribution is observed compared with the GHS made with nonporous microgels. This shows successful cell infiltration into the void spaces of porous microgels, as expected based on the successful cell infiltration into individual microgels. FIG. 30 shows the high viability of cells (>95%) in GHS made up of porous or nonporous GelMA microgels with no significant difference among the study groups, indicating the biocompatibility of porous GHS in vitro.

To further investigate the in vitro cell behavior in the GHS, the metabolic activity of cells is assessed using the PrestoBlue assay during cell culture. FIG. 30 presents the metabolic activity of cells on days 1, 4, and 7 after forming GHS using cell-microgel suspensions. On day 7, the assay shows the cell metabolic activity is at least tripled across all groups. While the overall growth in metabolic activity is expected because of the interparticle void spaces in GHS, increasing the transport of oxygen, nutrients, and cellular waste, the incorporation of porous microgels in GHS does not significantly alter the cell metabolic activity compared with the nonporous microgels. A reason for this observation may be that GHS enable sufficient metabolite diffusion despite the differences in void fraction and pore size among the study groups.

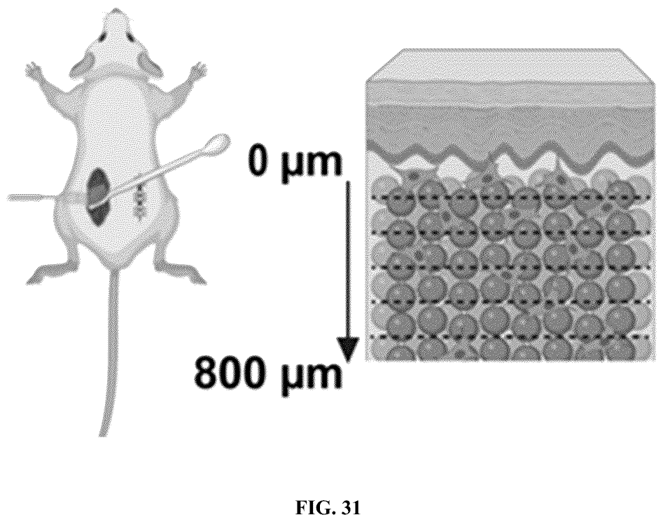

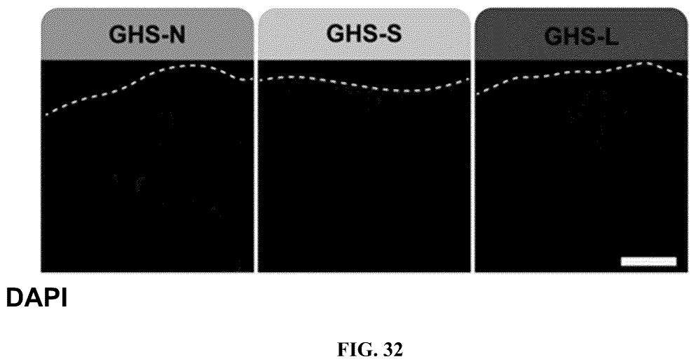

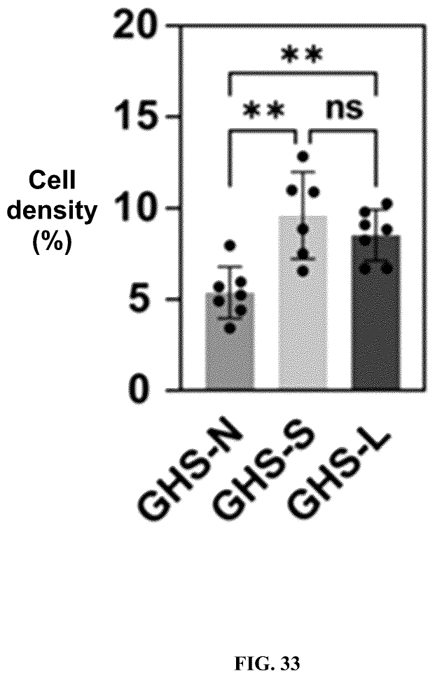

Cell infiltration is a critical stage in facilitating the integration of scaffolds with a host tissue and promoting tissue regeneration. We hypothesize that surpassing the void fraction limit of GHS-N using porous microgels may provide additional space and surface area, enhancing the recruitment of endogenous cells and thus facilitating the scaffold integration within the host tissue. To assess cell infiltration in GHS made up of porous or nonporous microgels, we subcutaneously implant the scaffolds into pockets, formed on the dorsal skin of mice, as schematically shown in FIG. 31. Following a two-week period, all scaffolds are explanted, and subsequent immunofluorescence and H&E staining analyses are conducted. FIG. 32 shows the extent of cell infiltration into scaffolds, as indicated by DAPI-stained cell nuclei in 7-10 μm-thick sections. Compared with GHS-N, GHS-S and GHS-L have higher cell density. The uniformity in cell distribution observed in GHS composed of porous microgels may be attributed to the pores at both inter-and intra-microgel levels, which is consistent with the in vitro results. The hierarchical pores enable the cells to occupy not only the void spaces among the microgels but also within the porous microgels. For the quantitative analysis of cell infiltration in the scaffolds, cell density is measured across different ROIs (length˜800 μm and height˜400 μm), starting from the tissue-scaffold interface, marked with a dashed line in FIG. 32. FIG. 33 presents cell density in the explanted samples, where the fraction of DAPI fluorescence area over the total area of ROI in GHS-S (9.6±2.4%) and GHS-L (8.5±1.4%) is significantly higher than that in GHS-N (5.4±1.4%). This shows an approximately 78% and 57% increase in cell infiltration in GHS-S and GHS-L, respectively, compared with GHS-N. We attribute the observed trend in cell density across the study groups to the differences in void fraction. Specifically, GHS-S and GHS-L have a ˜170% and ˜140% increase in void fraction compared with GHS-N, respectively. Such a significant increase in void fraction, provides more surface area for cell adhesion, thereby promoting higher cell infiltration into the scaffolds.

FIG. 34, show the quantitative analysis of cell infiltration in varying depths of scaffolds, measured using ROIs with length ˜400 μm and height ˜200 μm. Cell infiltration in all study groups within the first layer of scaffolds (˜200 μm) from the interface is significantly higher than the other layers. No statistically significant difference is observed between the remaining layers in each group. In the top layer, cell infiltration in GHS-N, GHS-S, and GHS-L is 46±13%, 61±19%, and 66±17% of the total cell area in each study group, respectively. The higher cell density observed in the first layer of GHS-S and GHS-L compared with the other layers within the same sample may be attributed to the higher void fractions in the porous microgels. This creates more void spaces in the first layer for cells to infiltrate before moving deeper into the scaffold. Although a gradual decrease in the cell infiltration is noted within the scaffolds (depth=200-800 μm) across all samples, no significant difference is observed across the remaining layers.

To compare cell infiltration at each depth among the study groups, the cell nucleus area at each interval (depth=200 μm) of GHS-S and GHS-L is normalized with the average cell area in the corresponding layer in GHS-N. It is noteworthy that the first two layers in GHS-S undergo ˜240% and ˜170% increases in cell infiltration compared with the same layers in GHS-N, respectively. Similarly, in GHS-L, the first two layers undergo a ˜230% and ˜135% increase in cell infiltration, respectively, compared with the corresponding layers in GHS-N. This increase is attributed to the additional void spaces within individual microgels, which facilitate endogenous cell infiltration in GHS. Moreover, to compare cell infiltration in GHS-S and GHS-L, the cell nucleus area in each depth of GHS-L is normalized with the average cell area in the corresponding layer in GHS-S. Although the normalized cell area does not significantly change across varying depths of the scaffolds, at least a ˜3% increase in cell infiltration is observed in the first three layers of GHS-S compared with GHS-L. This increase is possibly because of the larger median pore size of GHS-L, which facilitates cell infiltration into the void spaces of individual microgels.

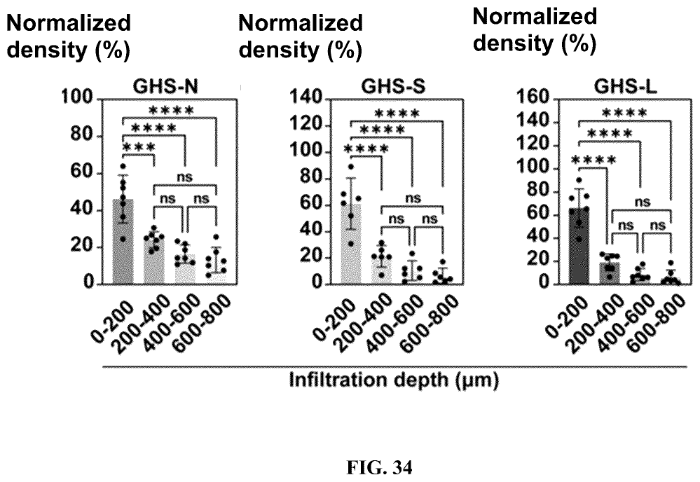

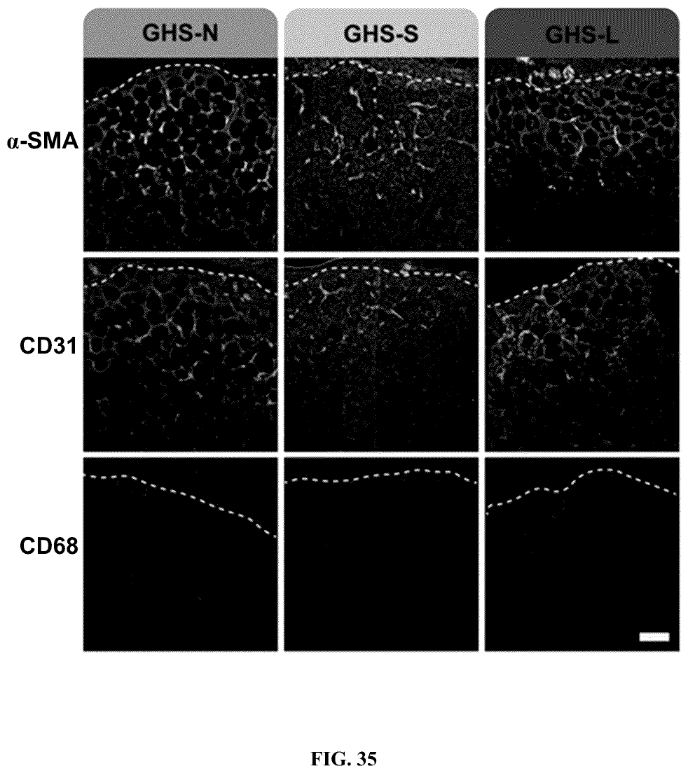

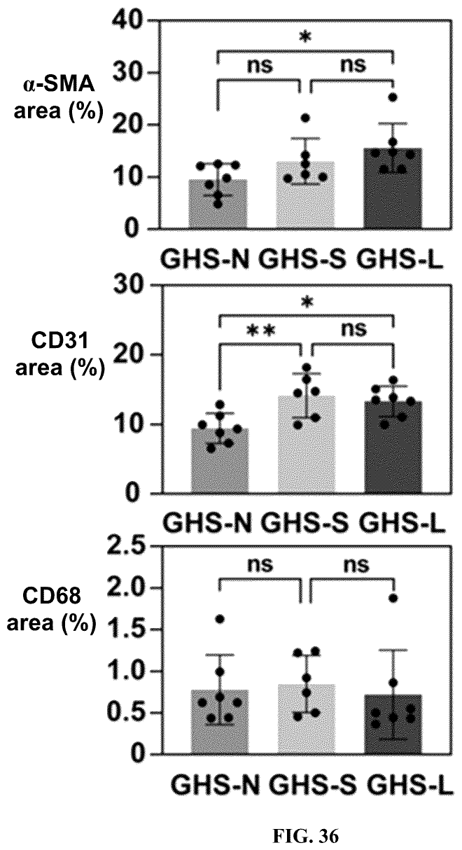

Subsequently, immunofluorescence staining is conducted to identify the type of cells that infiltrated the scaffolds, made up of porous or nonporous microgels. FIG. 35 presents the immunofluorescence images of varying cell populations, including endothelial cells, myofibroblasts, and macrophages, infiltrated into the scaffolds, which are stained by CD31, α-SMA, and CD68 markers, respectively. FIG. 36 presents the average of α-SMA-stained area normalized with the total ROI area. In GHS-L, myofibroblast infiltration is 15.6±4.7%, showing a ˜64% increase compared with GHS-N (9.5±3.0%), while no significant difference is observed between GHS-L and GHS-S (13.0±4.4%). FIG. 36 shows CD31-stained area in the GHS, made up of porous or nonporous microgels. Endothelial cell population is significantly higher in GHS-S (14.1±3.2%) and GHS-L (13.3±2.2%) compared with GHS-N (9.4±2.2%), showing 50% and ˜41% increase in infiltration, respectively. FIG. 36 presents the macrophage infiltrated area in varying study groups. While a higher stained area for CD31 and α-SMA is observed in GHS-S and GHS-L compared with GHS-N, no significant difference is observed for the macrophage infiltration across the study groups. Overall, the results imply that the increased void fraction in the GHS composed of porous microgels leads to higher cell infiltration compared with the GHS made up of nonporous microgels. Furthermore, the increased endothelial and myofibroblast recruitment underscores the potential of these scaffolds for tissue regeneration applications, where vascularization is critical.

### Example 3

### Cell Adhesion and Self-Assembly of Cell-Microgel Biohybrid Spheroids

### Materials

Novec™ 7500 Engineered Fluid was purchased from 3M (MN, USA). HUVEC and NIH/3T3 cells were purchased from ATCC (VA, USA). BRAND® microplates (BRANDplates®, inertGrade, low-binding, 96 wells, 330 μL, round bottom, transparent) were purchased from BRAND GMBH+CO KG (Germany). Corning® Matrigel® Matrix was purchased from Corning (NY, USA). HyClone characterized fetal bovine serum (FBS) and HyClone penicillin-streptomycin 100× solution (P/S) were purchased from Cytiva (UT, USA). Polydimethylsiloxane (PDMS, SYLGARD 184 silicone elastomer kit) was purchased from Dow Corning (MI, USA). Biopsy punches (1.5 mm with plunger system) were purchased from Integra Miltex (NY, USA). KMPR 1000 series photoresists were purchased from Kayaku Advanced Materials (MA, USA). EGM™-2 Endothelial Cell Growth Medium-2 BulletKit™ was purchased from Lonza (Switzerland). Ultra-pure Milli-Q water (electrical resistivity≈18 M (2 cm at 25° C.) provided from Direct-Q 5 UV remote water purification system, Millipore Corporation (MA, USA). Mesenchymal stem cell growth medium 2 was purchased from PromoCell (Germany). Disposable cell scrapers (Biologix, disposable, polyethylene, sterile, handle length: 180 mm, blade length: 18 mm), micro centrifuge tubes (Celltreat, 1.5 mL, clear, polypropylene), centrifuge tube (Celltreat, 15 mL, sterile), and cell strainer (Celltreat, 40 μm, polypropylene, sterile) were purchased from Neta Scientific Inc. (NJ, USA). Deuterium oxide (D₂O, deuteration degree 99.95%), gelatin (type A from porcine skin, gel strength ˜300 g Bloom), Methacrylic anhydride (MAA, contains 2,000 ppm topanol A as inhibitor, 94%), lithium phenyl-2,4,6-trimethylbenzoylphosphite (LAP, >95%), fluorescein isothiocyanate (FITC)-dextran (average molecular weight=2 MDa), trichloro (1H,1H,2H,2H-perfluorooctyl) silane (F-silane, 97%), 4-(4,6-Dimethoxy-1,3,5-triazin-2-yl)-4-methylmorpholinium chloride (DMTMM, ≥97.0%), and Human MSCs (Bone Marrow) were purchased from Sigma. Pico-Surf® (1% in Novec™ 7500) was purchased from Sphere Fluidics (UK). 1H, 1H, 2H, 2H-perfluoro-1-octanol (PFO) and a variety of cell culture and staining reagents including Gibco™ Dulbecco's phosphate buffered saline (DPBS, 1×, no calcium, no magnesium), Dulbecco's modified Eagle medium (DMEM), Trypsin-EDTA (0.25%, with phenol red), Pierce™ 16% formaldehyde (w/v, methanol-free), PrestoBlue™ cell viability reagent, LIVE/DEAD™ cell imaging kit (contains Calcein AM, cell permeant dye as live cell indicator and BOBO-3 Iodide as dead cell indicator), Alexa Fluor™ 647 hydrazide, Hoechst 34580, CellTracker™ Fluorescent Probes (Green CMFDA and Red CMTPX Dyes), and 4′,6-Diamidino-2-Phenylindole (DAPI) were purchased from Thermo Fisher Scientific (MA, USA). Dialysis membrane (12-14 kDa molecular weight cutoff) was purchased from Spectrum Laboratories (NJ, USA). Silicon wafers (100 mm diameter, 500 μm thickness, University Wafer, Inc., MA, USA). Sterile disposable filter units with (filter capacity 500 mL, pore size 0.2 μm), disposable pipetting reservoirs, plain microscope slides (thickness 1.0 mm, length×width 75 mm×25 mm), petri dishes (sterile, 60 mm×15 mm, polystyrene), and ethanol (200 proof/100%) were purchased from VWR (PA, USA).

### Methods—Gelatin Methacryloyl (GelMA) Synthesis

Gelatin methacryloyl (GelMA) was synthesized according to our previously established protocols. DPBS (400 mL) was heated to 50° C., and 20 mg of gelatin was dissolved via stirring at 200 rpm. MAA (16 mL) was then added dropwise to the mixture being stirred at 50° C. The reaction was stopped after 3 h by adding 400 mL of DPBS. The solution was then dialyzed against ultra-pure Milli-Q water for 10 days at 40° C. to remove unreacted MAA. A clear solution was obtained, sterile filtered using filtration units, and frozen at −80° C. Finally, the frozen GelMA was freeze-dried using Labconco FreeZone 4.5L-84 C Benchtop Freeze Dryer (Labconco Corporation, MO, USA) at a collector temperature of −82.4° C. under 0.009 mbar of pressure to yield a white solid.

### Methods—Proton Nuclear Magnetic Resonance (1H NMR) Spectroscopy

GelMA synthesis was confirmed by 1H NMR spectroscopy using a 500 MHz Bruker NEO instrument (MA, USA) at the Pennsylvania State University NMR facility. Gelatin and GelMA samples (40 mg each) were dissolved in 2 mL of D₂O, then heated at 37° C. for 2 h to ensure complete dissolution. Vinyl group peaks, indicative of methacryloyl modification and absent in gelatin, were identified between 5.5 to 6.5 ppm using TopSpin software (version 4.0.7, Bruker, MA, USA).

### Methods—GelMA Fluorescent Conjugation

To conjugate GelMA with a fluorophore, 1 g of GelMA was dissolved in 50 mL of DPBS under continuous stirring at 200 rpm at 40° C. until fully dissolved. Then, 22 mg of DMTMM was introduced into the mixture, followed by the addition of Alexa Fluor 647 hydrazide solution (48 μL). The conjugation reaction was allowed to proceed at 40° C. for 2 h. Then, the solution was dialyzed against Milli-Q water for 3 days at 40° C. to remove unreacted components. After dialysis, the solution was frozen at −80° C., followed by lyophilization to obtain the fluorescently conjugated GelMA in a solid form. The lyophilized product was stored in the dark.

### Methods—Microfluidic Device Fabrication

High-throughput step emulsification microfluidic devices were fabricated at the nanofabrication facilities at the Pennsylvania State University. Two-or three-layer master molds were fabricated on silicon wafers using the KMPR 1000 series as the negative photoresists. The first layer was spin-coated with KMPR 1005, KMPR 1025, or KMPR 1035 for the mold fabrication of small, medium, or large droplets, respectively, according to the manufacturer guidelines. This resulted in layer heights of 8, 27, or 60 μm, respectively. Following this, subsequent layers were deposited using KMPR 1035, designed to be 2-3 times the size of the anticipated droplet, to provide ample space for droplet formation and mobility. The devices were then fabricated using the PDMS by mixing the base and crosslinker at a ratio of 10:1, vacuum degassing, pouring onto the nanofabricated molds, degassing again, and curing at 80° C. for 2 h. The devices were then punched with a 1.5 mm biopsy punch for the inlets and outlets, bonded to a glass microscope slide after air plasma treatment at 400 mTorr for 45 s, followed by an F-silane (2 vol % in Novec engineered fluid) treatment. The treated devices were rinsed with Novec engineered fluid and then heated in an oven at 80° C. for 30 minutes to evaporate residual oil.

### Methods—Microgel Fabrication

Freeze-dried GelMA was dissolved in DPBS, containing a photoinitiator (LAP at a final concentration of 0.1% w/v), at 40° C. to prepare a 5% w/v aqueous GelMA solution. A mixture of Novec engineering fluid, containing 2% v/v Pico-Surf™ surfactant, was used as the oil phase for the small and medium droplets, and a 0.5% v/v surfactant in the same oil was used for the large droplets. The droplet fabrication system was kept at around 35-40° C. using a space heater. Then, droplets were maintained at 4° C. overnight to form physical crosslinked microgels. Microgels were then photocrosslinked via light exposure (wavelength=395-400 nm, intensity=15 mW cm⁻²) for 5 min, followed by adding an equal volume of PFO (20% v/v in Novec engineered fluid), vortexing for 5 s, and centrifuging at 300×g for 15 s to remove the oil and surfactant. To ensure no residual oil or surfactant remained, the microgel suspension was rinsed with an equal volume of DPBS, vortexed, and centrifuged at 300×g for 15 s.

### Methods—Cell Culture

NIH/3T3 mouse fibroblast cells, primary HUVEC, or MSC were cultured in DMEM (supplemented with 10% v/v FBS and 1% v/v antibiotics), EGM-2, or complete mesenchymal stem cell growth media, respectively. The medium was refreshed every other day, and the cells were passaged when they reached 80% confluency, typically twice a week. A standard cell culture incubator (Eppendorf, Hamburg, Germany) was used to culture cells under a 5% v/v carbon dioxide (CO₂) atmosphere at 37° C. The cells were trypsinized (detached from the culture dish) using a 0.25% trypsin-EDTA solution, followed by counting using an automated cytometer (Countess 2, ThermoFisher Scientific, MA, USA).

### Methods—BHS and Cell Spheroid Formation

To form BHS without geometric constraints, crosslinked GelMA microgels were packed at 3000×g and then pipetted into a petri dish (60 mm) using positive displacement pipette (MICROMAN E M100E, Gilson Company, Inc., OH, USA). To ensure similar total surface area, 10, 27, or 50 μL of small, medium, or large packed microgel suspension was used. Cell suspension (3 million cells per petri dish) was also added in 5 mL of media, resulting in a cell concentration of 600,000 cells mL⁻¹. The microgels and cells were mixed by gentle pipetting. To form BHS under geometric constraints, GelMA microgels packed at the similar condition mentioned above. Then, microgels and cells were mixed, while maintaining a constant cell density of 600,000 cells mL⁻¹ and varying the microgel amount according to their sizes, 2, 5.4, or 10 μL mL⁻¹ for small, medium, or large, respectively. The microgel-cell suspensions were gently pipetted and transferred into disposable pipetting reservoirs. Then, 200 μL of the mixture was added to each well of a U-bottom 96-well plate. The culture was maintained for up to one week, with the media being refreshed daily. Cell spheroid is formed similar to BHS, but microgel-free.

### Methods—Analysis of BHS and Cell Spheroid Formation

A CytoSMART Lux2™ cell imaging microscope (CytoSMART Technologies, Netherlands), equipped with a 10× objective, was used to image BHS formation (unconstraint) over time. Images were acquired at 5-min intervals for 72 h and used to determine morphological and kinetic parameters. The area of each BHS was calculated using the ImageJ software (Fiji, version 1.54f, NIH, MD, USA). The attachment of the cells to both microgels and other cells during BHS and cell spheroid formation were assessed by tracking them using a custom-written Mathematica code (Wolfram Mathematica, version 13.3, Wolfram Research, IL, USA). BHS formation in the U-bottom plates (i.e., constraint geometry) were imaged using Incucyte® S3 Live-cell Analysis system (version 2022A, Sartorius, Germany) at Sartorius cell culture facilities, the Pennsylvania State University, with brightfield, and green-fluorescence protein (GFP) channels. Images acquired every 30 min for 5 days using a 4× objective and analyzed using the Incucyte® spheroid analysis software module (version 2022A, Sartorius, Germany).

### Methods—Microgels Tracking

Microgel particles were tracked using a custom Python script using the trackpy library. The stacked images were loaded, and particles were initially detected in the first frame. The parameters for particle detection, including diameter and minimum mass threshold, were optimized based on the microgel sizes to ensure accurate identification. Tracking of particles was performed frame by frame. The trajectories were visualized, and short-lived tracks persisting for fewer than 10 frames were filtered out to ensure reliability in the subsequent analysis.

### Methods—Quantification and Analysis of Single Cells in Aggregate Formation Kinetics

The attachment of the cells to both microgels and other cells during BHS and cell spheroid formation was assessed by tracking them using a custom-written Mathematica code (Wolfram Mathematica, version 13.3, Wolfram Research, IL, USA). For each microgel size, an exponential decay function was fitted to the number of individual cells over the initial 10 h. This fitting used the exponential decay model as

$$N ( t ) = {N}_{0} {e}^{- \frac{t}{τ}} ,$$

where N(t) is the number of remaining individual cells at time t, N₀ is the initial number of cells, and τ is the characteristic decay time.

### Methods—Porosity Characterization

BHS were formed in U-bottom wells (constraint) for 3 days, followed by incubation in a FITC-dextran solution (Mw≈2 MDa, 30 μM in DPBS) for 10 minutes to fill voids. Porosity was determined from 3D Z-stacked images (volume of interest: 149.66×149.66×68.21 μm³ in X, Y, and Z, respectively) acquired using a Leica STELLARIS 5 confocal microscope (Leica Microsystems, Germany). The LAS X (version 5.0.3, Leica Microsystems, Germany) software calculated void volume fractions by comparing stained interstitial space against total volume.

### Methods—Cell Staining Procedures

Cells were visualized using fluorescent staining. BHS and cell spheroid fixed with 4% paraformaldehyde for 2 h at room temperature, followed by permeabilization with 0.2% Triton X-100 in DPBS for 2 h. Then, aggregates were washed with DPBS at least five times, each for 10 min. For actin filament staining, Phalloidin Alexa Fluor 488 was applied (1:40 volume ratio in DPBS) and incubated overnight in darkness at 4° C. Nuclear staining was achieved using DAPI (1:1000 volume ratio in DPBS) for 2 h. Live cell labeling was performed using CellTracker Green CMFDA and Red CMTPX, according to the manufacturer's protocol on 2D cultured cells. Additionally, live cells nuclei stained with Hoechst (1:2000 volume ratio in PBS) for 10 min.

### Methods—Scanning Electron Miscroscopy (SEM)

The surface morphology of aggregates was investigated using a scanning electron microscope (Quanta 250 ESEM, Thermo-Scientific, OR, USA) at the materials characterization lab at the Pennsylvania State University. Cell spheroid or BHS were formed in 3 days, then fixed in 4% v/v of paraformaldehyde for 3 h. The fixed samples were rinsed at least five times with DPBS and subsequently immersed in a gradient of ethanol solutions with concentrations ranging from 15% to 100% (v/v in milli-Q water). The samples were then dried using a critical point dryer (CPD300, Leica EM, Germany) to ensure complete removal of any fluid. Finally, the samples were sputter-coated with iridium (thickness ˜2-5 nm, Emitech K575 Turbo sputter coater, E.M. Technologies, UK) and imaged with a beam current of 91 pA under an accelerating voltage of 5 keV, using an Everhart-Thornley detector (ETD) in Secondary Electron (SE) mode.

### Methods—Cell Metabolic Activity Assessment

The cell metabolic activity solution was prepared by adding Presto Blue™ cell viability solution to DMEM (serum-free) in a 1:9 volume ratio. The BHS cultured on a planar rigid substrate (unconstraint) were removed using a cell scraper, then moved into a 15 mL centrifuge tube. The tube was centrifuged for 5 minutes at 300×g. After discarding the supernatant, 3 mL of metabolic activity solution was added to the centrifuged BHS. The tubes were wrapped in aluminum foil and incubated for 4 h in a cell culture incubator at 37° C., with 5% CO₂. Then, the tubes were re-centrifuged at 300×g for 5 minutes and the supernatant was collected for analysis. Fluorescence intensity was recorded using a microplate reader (Tecan Infinite M Plex, Switzerland) at 560 nm excitation and 590 nm emission. The metabolic activity of BHS formed in U-bottom well-plates (constraint) was measured using a metabolic activity solution of Presto Blue™ cell viability solution mixed with equal volume of serum-free DMEM. Then, 50 μL of this stock solution was added to each well. The plate incubated at 37° C., with 5% CO₂, for 4 h, followed by analysis using the aforementioned device/condition.

### Methods—Cell Viability Assessment

Using a cell scraper, all cells, microgels, and/or formed aggregates were detached from the petri dish and transferred to a centrifuge tube. The tubes were centrifuged at 300×g for 5 min, followed by discarding the supernatant. Cell viability within the BHS or cell spheroid was assessed using a two-color fluorescence LIVE/DEAD™ cell imaging kit. The imaging solution made from Calcein AM as live cell indicator and BOBO-3 Iodide for staining dead cells was prepared according to the manufacturer protocol, followed by adding 1 mL into each centrifuge tube and resuspending via gentle pipetting. The centrifuge tubes were then wrapped in an aluminum foil and placed under the biosafety cabinet at room temperature for 30 min and imaged using the Leica DMi8 fluorescence microscope (THUNDER imaging systems, Leica Microsystems, Germany). The live cells channel was set to 470 nm excitation and 510 nm emission wavelengths. The dead cell channel was set to 550 nm excitation and 610 nm emission wavelengths. Images analyzed using ImageJ software (Fiji, version 1.54f, NIH, MD, USA), and cell viability was reported as the number of live cells, over total number of cells.

### Methods—Angiogenic Sprouting Assay

Sprouting assays were performed using HUVEC cell spheroids or BHS. Matrigel was thawed overnight at 4° C., coated onto a 48-well plate, and cured at 37° C. for 4 h. Assemblies were cultured on top of the Matrigel for 3 days, stained with the two-color fluorescence LIVE/DEAD™ cell imaging kit, and imaged using the Leica DMi8 fluorescence microscope (THUNDER imaging systems, Leica Microsystems, Germany).

### Methods—Fusion Test

BHS or cell spheroid aggregates were prepared in U-bottom wells (constraint) for 3 days. Four aggregates of the same type (either BHS-S, BHS-M, BHS-L, or cell spheroid) were transferred and placed in proximity within a U-bottom well. Media was refreshed daily. Brightfield images were captured every day using an EVOS™ XL Core microscope (Thermo Fisher Scientific, MA, USA). Area of fused aggregates was measured using ImageJ software (Fiji, version 1.54f, NIH, MD, USA), and results were reported accordingly.

### Methods—Formation of mm-Scale Tissue-Like Structures

BHS were cultured on a non-constraining, rigid planar substrate for 3 days. Subsequently, these aggregates were detached using a cell scraper and passed through a cell strainer (40 μm pores) to remove unbound cells and debris, ensuring retention of BHS on the strainer due to their size. The collected aggregates were then placed into a custom-designed PDMS cylindrical mold (12 mm diameter, 3 mm height), ensuring close contact among them. Following an additional 3 days of culturing with daily media refreshment, a large mm-scale scaffold was formed, which could be readily removed using a spatula.

### Methods—GelMA Bulk Hydrogel Scaffold Fabrication

Bulk GelMA hydrogel scaffolds were fabricated using a two-step crosslinking process, mirroring the method for microgel fabrication, to ensure that they share similar physicochemical properties. Scaffolds were prepared by dissolving GelMA in a 0.1% w/v LAP solution in DPBS at a concentration of 5% w/v at 40° C. until GelMA dissolved completely. This homogeneous GelMA solution was poured into cylindrical acrylic molds (diameter=10 mm, height=1 mm). To prevent dehydration, molds were placed in a dark, custom-built humidity chamber. After cooling at 4° C. overnight for physical crosslinking, hydrogels underwent photocrosslinking under light (wavelength=395-400 nm, intensity=15 mW cm⁻²) for 5 min, resulting in stable bulk hydrogel scaffolds.

### Methods—Compression Test

The mechanical properties of GelMA bulk hydrogel scaffolds were assessed using an Instron mechanical tester (Model 5542, Instron Corporation, MA, USA). Samples were pre-incubated at 37° C. for 2 h, punched into dimensions of 8 mm in diameter and 1 mm in height, then subjected to uniaxial compression at a controlled displacement rate of 1 mm min⁻¹. Testing continued until 70% strain or sample failure. Stress-strain curves were recorded, and the compressive modulus was calculated from the initial linear region, between 0 and 10% strain.

### Methods—Viscoelastic Properties Assessment

Viscoelastic properties of GelMA hydrogel scaffolds and tissue-like structures were examined using a rotational rheometer (AR-G2, TA Instruments, DE, USA) with parallel plates (8 mm top plate and 20 mm bottom plate, both sandblasted stainless steel). Samples were punched to match the upper plate diameter for full coverage, then were incubated at 37° C. for 2 h. Rheological tests (performed at 37° C.) included an oscillatory amplitude sweep from 0.1% to 100% strain at a constant frequency of 1 rad s⁻¹ to identify the linear viscoelastic region (LVR), and a subsequent frequency sweep at 0.1% strain, from 0.1 to 100 rad s⁻¹. The storage modulus (G′) and loss modulus (G″) were evaluated to assess the sample viscoelasticity.

### Methods—Statistical Analyses

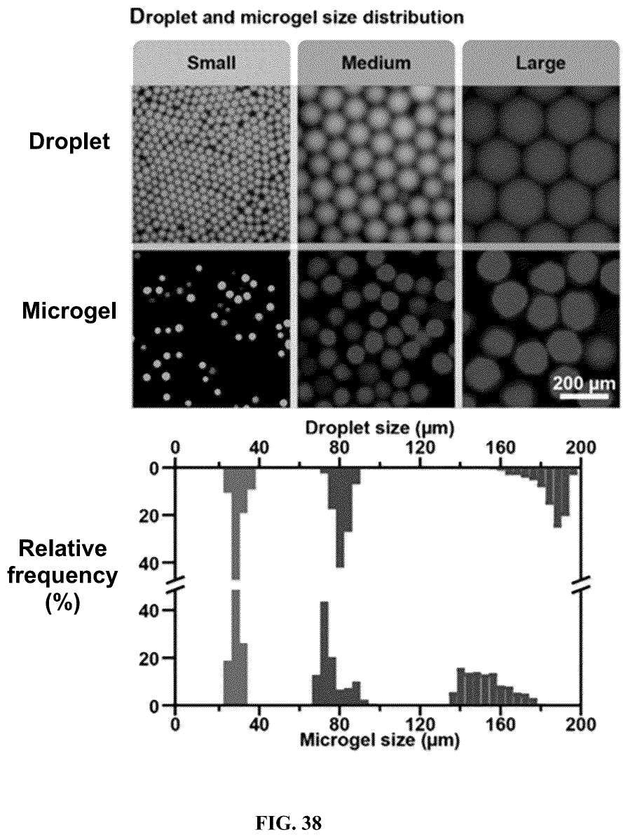

Experiments were conducted with at least three repeats. Data points in all figures represent independent repeats, except for FIG. 38, where all technical replicates within the ≥3 independent repeats are shown to represent the data distribution. Normally distributed data were analyzed using t-tests, or either ordinary or repeated measures (RM) one-way/two-way analysis of variance (ANOVA), with significance determined by Tukey's post-hoc multiple comparison test. Non-normally distributed data were assessed using the Kruskal-Wallis test followed by Dunn's post-hoc multiple comparison test. All statistical analyses were performed using GraphPad Prism (version 9.5.0). Groups with p-value below 0.05 were considered significantly different, indicated by *p<0.05, **p<0.01, ***p<0.001, ****p<0.0001.

### Results and Discussion

### Cell Adhesion and Migration Device Drive Snowballing-Like Assembly of BHS

Cells are provided with microscale ECM-mimetic substrates, gelatin methacryloyl (GelMA), a protein-based biopolymer, which is synthesized via reacting gelatin and methacrylic anhydride. GelMA is a photocrosslinkable, biodegradable biopolymer with tunable physicochemical properties. Upon light exposure, a photoinitiator (PI)-containing GelMA biopolymer solution forms a chemically crosslinked hydrogel, which is stable at physiological conditions (e.g., 37° C.). GelMA contains amino acid sequences, such as RGD peptides, that promote cell adhesion by interacting with integrins on the cell surface.

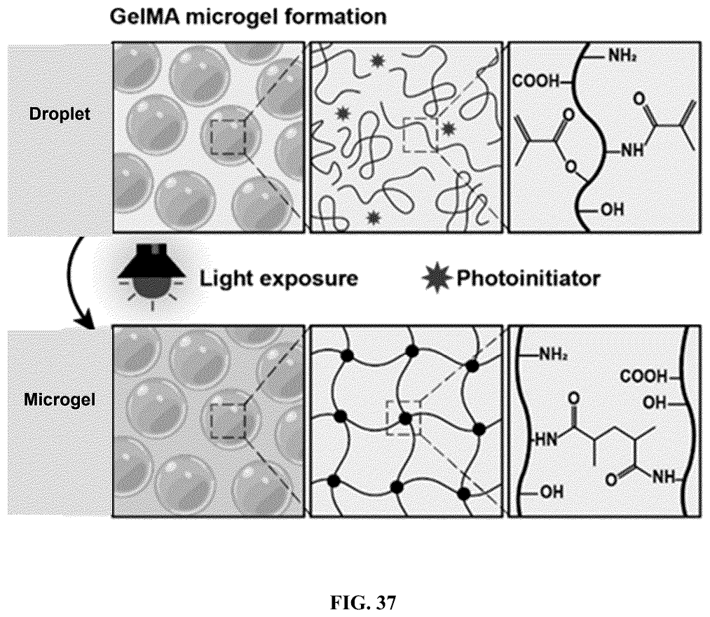

GelMA microgels with a controlled size are fabricated using step-emulsification microfluidic devices, which provide ECM-mimetic substrates for cell adhesion. By adjusting the step sizes of the microfluidic devices, droplets of varying diameters are produced according to an established method. These droplets are then converted to stable microgels through the free-radical photopolymerization of vinyl groups (FIG. 37). FIG. 38, shows the GelMA droplets, corresponding crosslinked microgels, and their size distribution. The average droplet diameter was 29±3 (small), 81±4 (medium), or 183±11 μm (large). Microgels undergo size reduction during photocrosslinking to 28±2, 76±6, and 152±10 μm for small, medium, and large, respectively. This reduction in microgel size post-photocrosslinking originated from the GelMA network densification, which leads to water expulsion and volume decrease. The sizes of these microgels are selected to approximate or exceed the sizes of NIH/3T3 fibroblast cells (measured diameter˜15±2 μm in a cell suspension).

Mixing the microgels with the NIH/3T3 murine fibroblast cells on a planar non-adhesive substrate prompts cell-cell and cell-microgel interactions. As schematically shown in FIG. 39, cell-cell interactions are facilitated by homophilic cadherin-cadherin binding and integrin-mediated binding to ECM components, such as fibronectin recycled by the cells. Integrin-mediated binding between cells and GelMA microgels, specifically RGD peptides and fibronectin, drives cell-microgel interactions. FIG. 40 shows the co-culture of microgels and fibroblast cells. The top row schematically represents the three-step assembly process: (i) initial mixing of cells and microgels, (ii) early-stage attachment of cells to microgels because of the adhesive moieties on GelMA, and (iii) the formation of two distinct aggregate types. The bottom row shows the pseudo-colored microscopy images of actual microgel-free cell spheroids, arising from cell-cell interactions, and BHS, which form through cell-microgel assembly.

### Microgel Size Regulates the Snowballing Kinetics and Terminal BHS Size

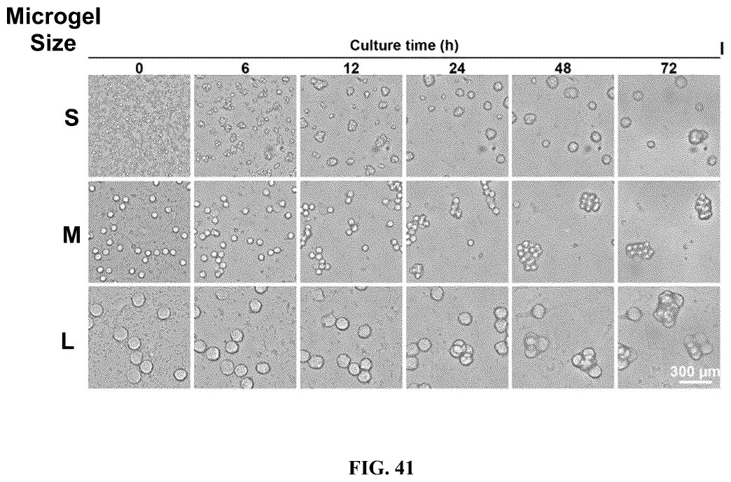

FIG. 41 shows the culture of fibroblast cells with microgels of three different sizes on a planar surface for 72 h, resulting in BHS formation. As cell-microgel interactions drive the aggregation and BHS formation, which depend on the accessible microgel surfaces, the total microgel surface area was maintained nearly constant in all the experiments. The total projected areas for small, medium, and large microgel suspensions are 13.7±0.9%, 14.8±3.0%, and 13.5±5.0%, respectively, showing no statistically significant differences. BHS formation occurs primarily within 24 h, but the aggregates continue to move, connect, and merge upon contact with much slower rates. Interestingly, we observe that cell spheroids are simultaneously formed in microgel-free regions, which may subsequently adhere to BHS upon contact. Control groups consisting of only microgels or cells are also monitored over time. Cell-free microgels do not undergo any significant movement or aggregate formation, showing cell mobility as the exclusive driving force for the aggregation. In contrast, the control comprising only cells undergo cell spheroid formation over time.

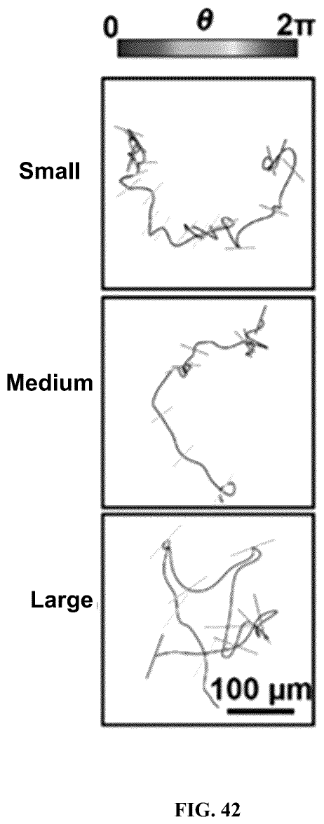

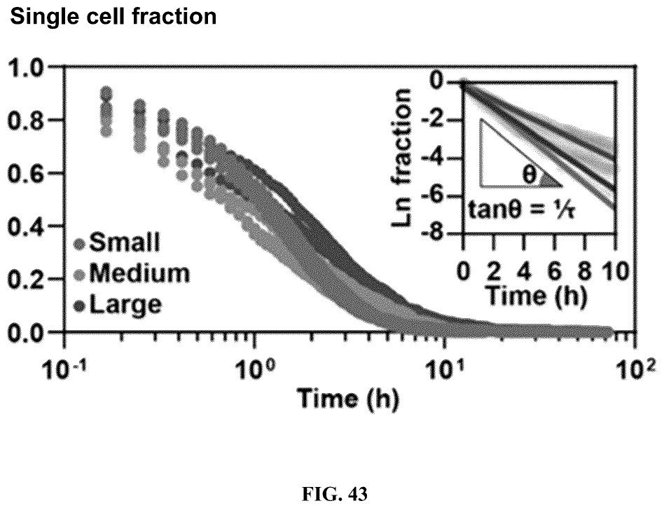

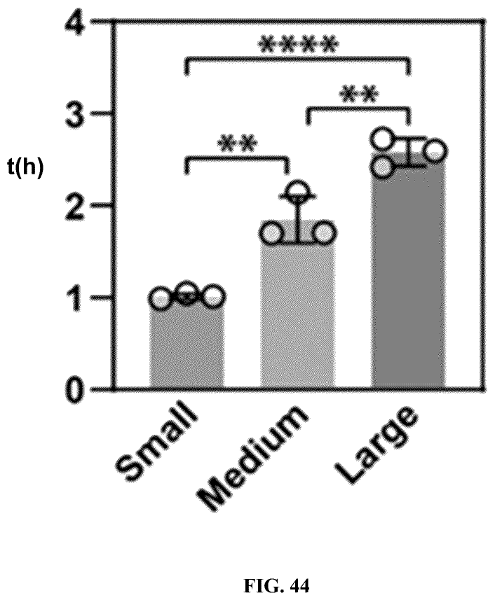

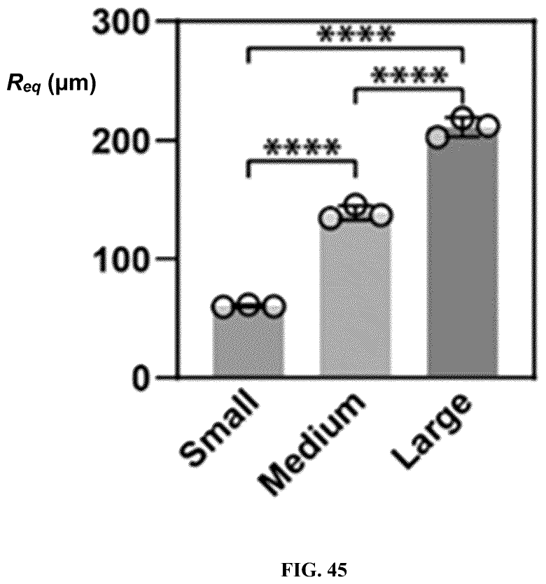

Our experimental observations indicate that the differential affinities between cells, microgels, and substrate are the primary drivers of BHS formation. The cells exhibit a higher integrin-mediated affinity for microgel surfaces than the untreated substrate. Since microgels are not self-adherent, the cells act as essential binding agents in the cell-microgel aggregation process (bio-glue). As a cell migrates from the substrate to microgels, its adhesion and contractility drive both translational and rotational motions of the growing aggregate, resembling to a snowballing process, facilitating the 2D to 3D transition of cell-microgel assemblies. Such rotational movement is evidenced by the dynamically changing angle of the line vector connecting two constituent microgels within the same spheroid, relative to the fixed coordinates of the culture plane, as shown in FIG. 42. To track the kinetics of BHS formation, we quantify the ratio of remaining individual cells due to aggregation depletion over time, as shown in FIG. 43. We observed a rapid exponential decline in the number of individual cells, dropping to <1% within approximately 10 h in all cases, indicating a nearly complete integration of all cells into aggregates. Under the same total surface area (Sₜₒₜ) of microgels, the characteristic decay time (τ) increases with microgel radius (FIG. 44), where τ is the time needed to reduce the number of individual cells to 1/e of its initial value. Furthermore, the terminal size of BHS (after 72 h) is quantified, as presented in FIG. 45, by measuring their equivalent radius R_{eq}=\sqrt{A/π}, where A denotes the projected area of an aggregate. FIG. 45 also shows that R_{eq} increases with microgel size and that the number density of stable BHS is higher for smaller microgel size. Overall, the microgel size determines the final aggregate size, which is kinetically arrested at long culture times (>10 h).

### BHS Architecture is Tailored by Geometric Constraints

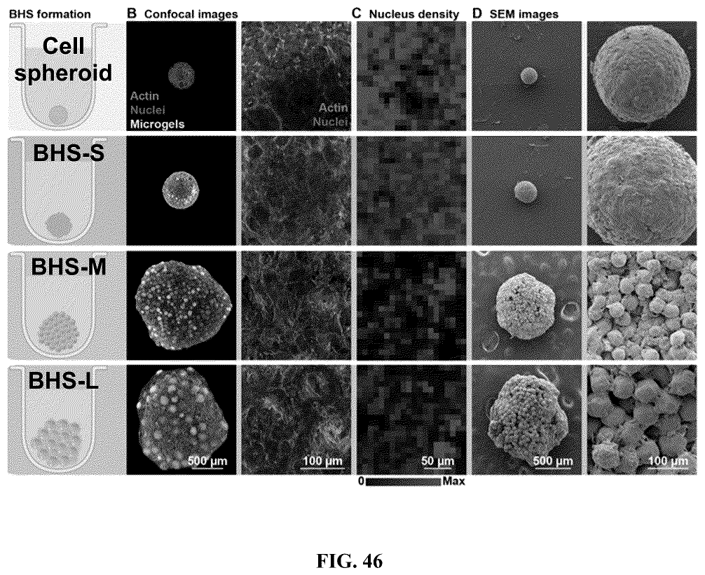

Cell-microgel culture on a flat, untreated substrate results in irregularly shaped BHS and cell spheroids with a diverse size distribution. Thus, an experimental system to enable single, large BHS formation with pre-determined cell and microgel number density is necessary to further assess microgels impact on cell behavior and aggregate morphology/porosity. In contrast to unconstraint, freely formed cell-microgel assemblies on a flat surface, BHS with a more controlled and regular shape are formed by mixing the cells and microgels in a low-attachment U-bottom well-plate. BHS-S, BHS-M, and BHS-L, or cell spheroid are formed using small, medium, and large microgels, or microgel-free cells, respectively, as schematically shown in FIG. 46. Confocal images of BHS and cell spheroids show that the cells are elongated and spread among the microgels in BHS-M and BHS-L, while in BHS-S and cell spheroids they remain more compact (FIG. 46). Moreover, FIG. 46 shows that the distance among cell nuclei is smaller in the cell spheroid and BHS-S compared with BHS-M and BHS-L, highlighting the increased cell compactness of the former aggregates.

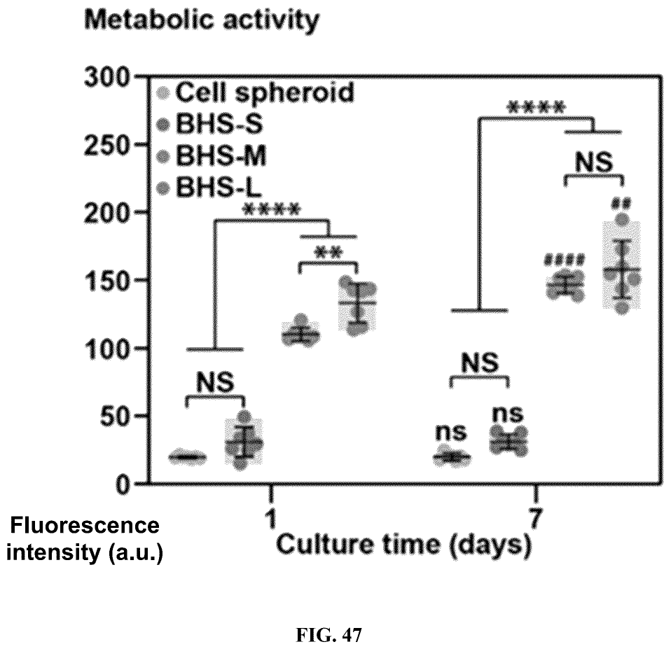

Scanning electron microscopy (SEM) images of aggregates in FIG. 46 show that cell spheroid and BHS-S are smaller and more spherical in shape compared with BHS-M and BHS-L. Compactness in cell spheroids correlates with sphericity, as the formation of cell spheroids depends on collective forces exerted on cells. In FIG. 47, we measure the metabolic activity of cells within aggregates on days 1 and 7, keeping a constant initial cell density.

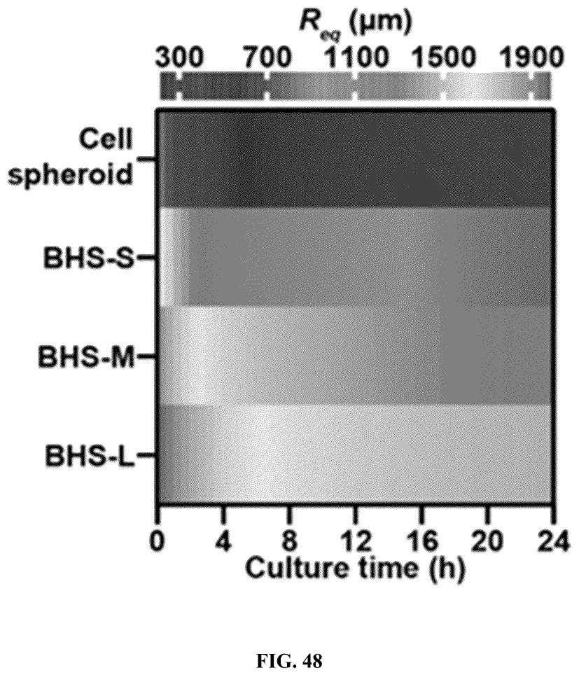

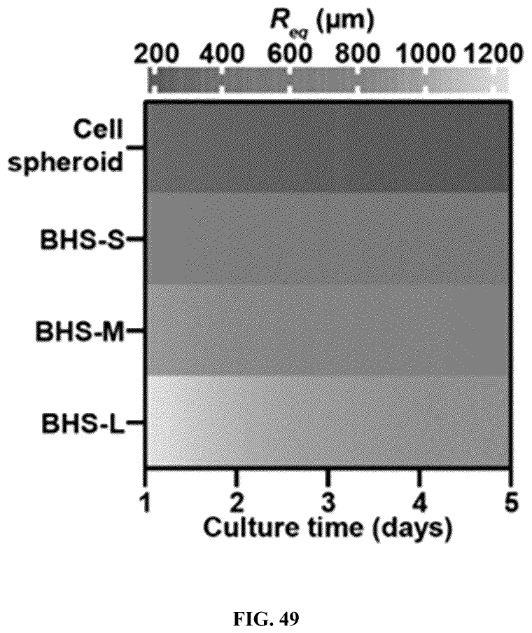

To analyze the dynamic formation of geometrically constraint cell spheroids and BHS over time, we acquired microscopic images of BHS and cell spheroid formation throughout a 5-day period. The control experiment with cell-free medium microgels did not result in any aggregate formation. Analyzing aggregate sizes over time highlights a trend: larger microgels need more time to reach a plateau in size, because at a similar cell attachment rate (FIGS. 42-44), larger microgels experience higher static friction and viscous forces due to an increased size and surface area. The most substantial BHS size change occurs within the initial 24 h (FIG. 48). The R_{eq} heatmap in FIG. 49 presents long-term size changes over five days of microgel-cell culture, showing that the change in size is microgel size dependent and eventually becomes kinetically arrested. BHS-S reached a stable size within 2 days, BHS-M required 3 days, and BHS-L took 4 days to attain size stability. Additionally, cell spheroid size was stabilized after 4 days, and the control group containing solely microgels had no size change throughout the experimental period.

Cells serve as both motors and adhesives agents in the BHS formation process; thus, we investigate their effects on BHS formation by manipulating the initial cell seeding density and monitoring BHS-M formation. Evaluations through both brightfield and fluorescence imaging show a notable deceleration in BHS formation when the cell density decreases. As the cell count decreases, there is a shift towards an extreme case resembling cell-free microgels. In these cases, the necessary driving force for assembly, namely cell-hydrogel interfacial interactions, may be compromised. Assemblies formed with fewer than 30,000 cells after a 5-day period lack sufficient cohesion, resulting in disintegration during handling, e.g., pipetting.

### BHS Formation Extends to Endothelial and Mesenchymal Stem Cells and Promote Angiogenic Sprouting

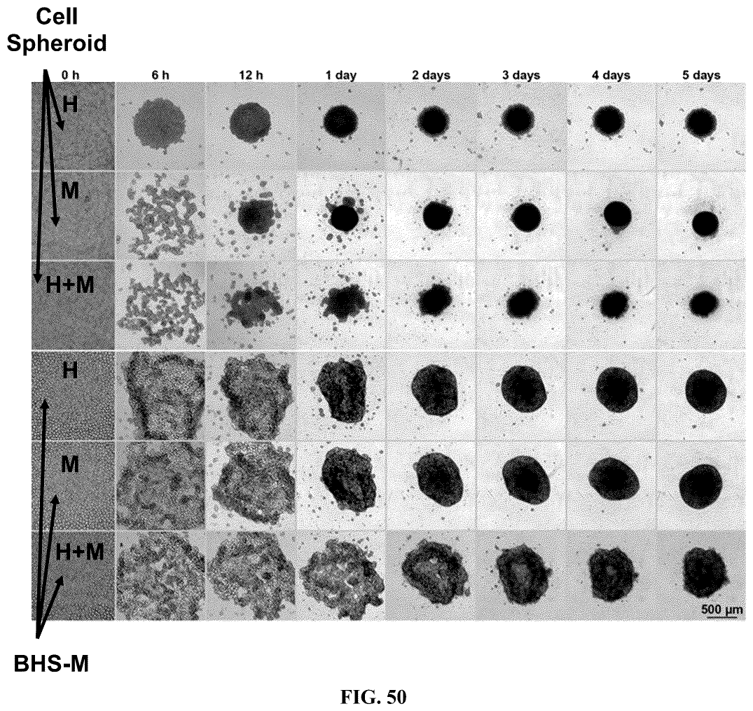

To extend our “snowballing-like” BHS formation beyond contractile NIH/3T3 fibroblasts, we investigate whether primary human umbilical vein endothelial cells (HUVECs), mesenchymal stem cells (MSCs), and their mixtures can form similarly robust 3D assemblies with GelMA microgels (FIG. 50). We formed six groups of cells-only or cell-microgel assemblies in geometrically constrained wells: (i) cell spheroids of HUVECs, MSCs, or HUVEC+MSC, and (ii) their corresponding BHS, formed by mixing each of these cell populations with GelMA microgels.

Initially (0-12 h), HUVEC-only spheroids rapidly adopt a round shape, similar to our earlier observations with fibroblasts, whereas MSC-only and HUVEC+MSC spheroids show incomplete compaction. This difference arises from robust HUVEC homophilic cell-cell adhesion (e.g., via VE-cadherin), which drives early spherical assembly. In contrast, MSC relies more on de novo ECM secretion beside homophilic contacts (e.g., via N-cadherin or Cadherin-11), resulting in slower aggregation.

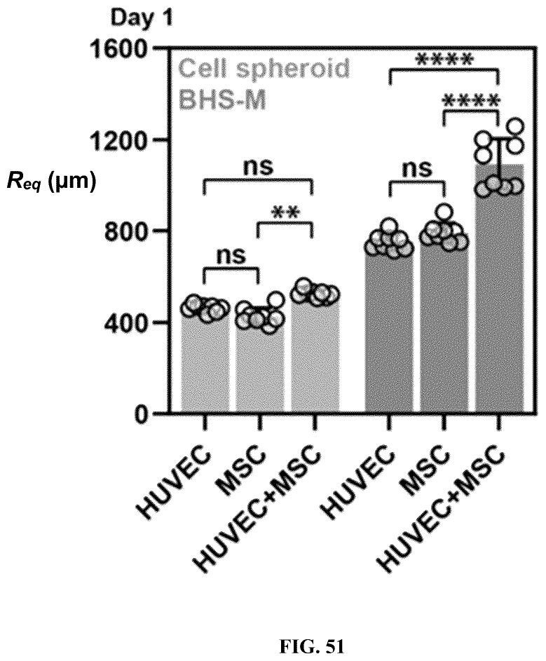

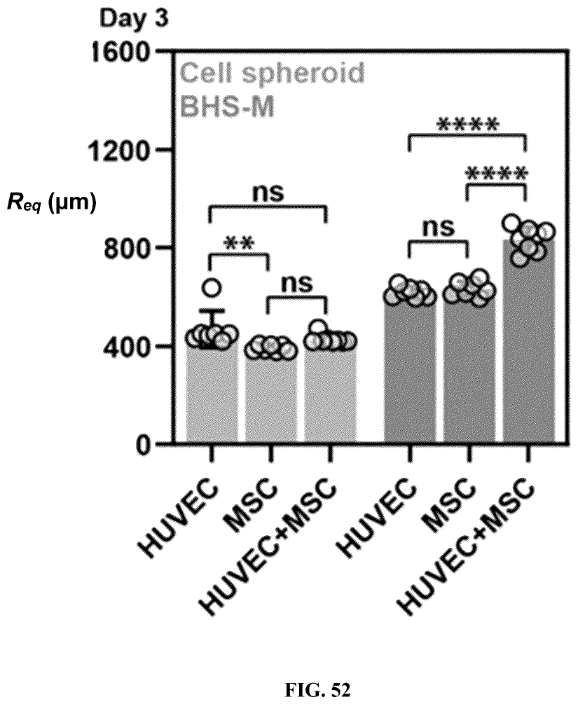

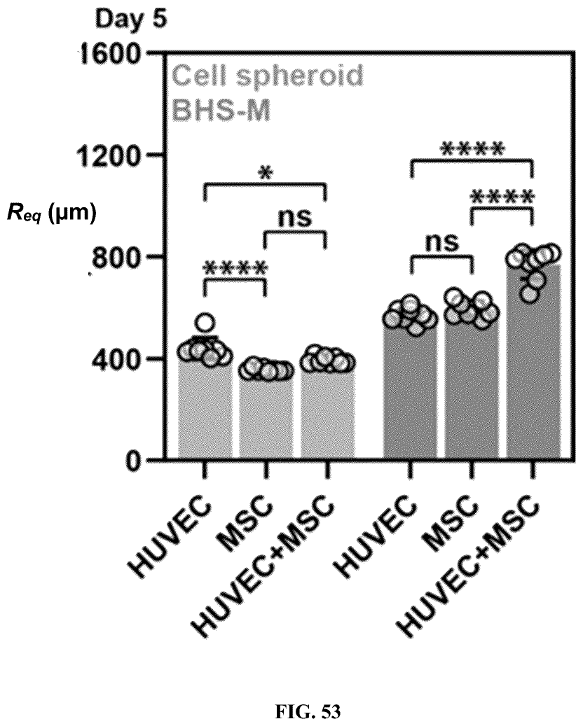

FIG. 51 captures the differences in equivalent radius (R_{eq}) on day 1: HUVEC spheroids appear as the largest among the cell-only aggregates, whereas the HUVEC+MSC BHS form the largest assembly among the BHS groups. We attribute this behavior to the heterotypic interactions between two cell types and the microgels, as well as the potential formation of early vessel-like structures—previously reported in HUVEC-MSC co-culture with GelMA—that collectively drive cell spatial reorganization which may be more voluminous. By days 3 and 5 (FIGS. 52-53), HUVEC spheroids maintain a relatively larger equivalent radius than MSC or HUVEC+MSC spheroids, likely because HUVECs, especially in pro-angiogenic media, favor hollow or less-dense spheroid architectures and secrete less ECMs than MSCs and fibroblasts. In contrast, MSC-only and HUVEC+MSC spheroids progressively densify, influenced by continued ECM deposition. Meanwhile, the HUVEC+MSC BHS persistently feature the largest size among the BHS conditions. Overall, these results indicate that BHS assembly extends beyond highly contractile fibroblast models to other cell types, such as HUVECs and MSCs, although the final size and aggregation kinetics vary.

We further evaluate the functional outcomes of these HUVEC-based assemblies by examining angiogenic sprouting. Compared with conventional HUVEC spheroids, which yield minimal outgrowth because of dense cell packing and inhibited nutrient diffusion, HUVEC BHS undergo substantially more pronounced vascular-like branching. This likely results from the microgel-mediated porosity that supports nutrient transport and cellular metabolism, thereby promoting angiogenic sprouting in vitro.

### BHS as a Versatile Building Block for Large Tissue-Like Structures In Vitro

Current hydrogel-based in vitro tissue models larger than the molecular diffusion limit (e.g., hundreds of microns) require perfusable blood vessels or channels to overcome the diffusion limit of oxygen and metabolite and maintain cell viability. Here, we assess BHS as building blocks for developing mm-scale tissue-like structures without requiring blood vessels. A critical criterion in developing large tissues in vitro is cell viability throughout the construct. We have shown that BHS made up of medium microgels have significantly higher cell viability and metabolic activity compared with the microgel-free cell spheroid.

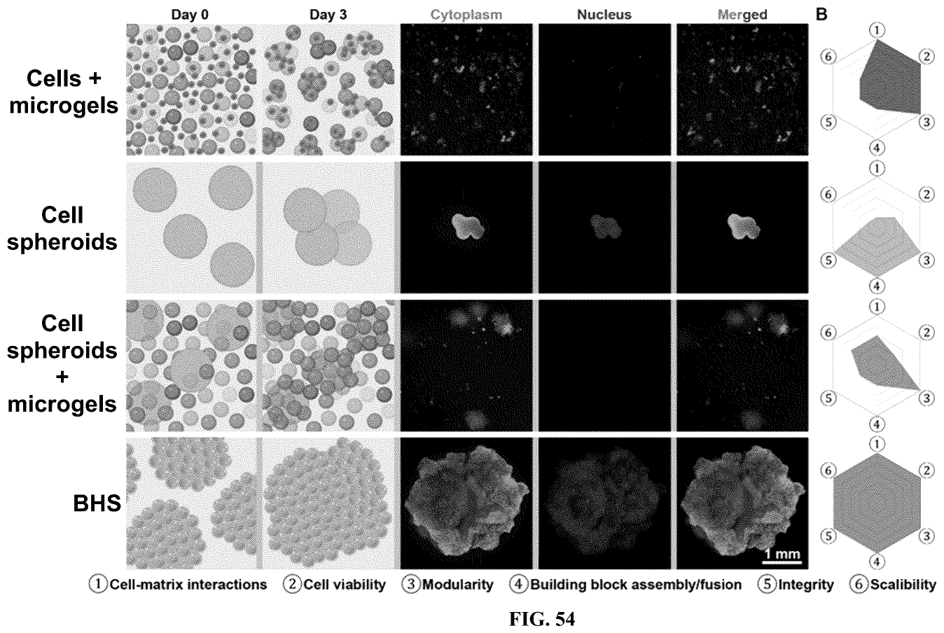

In FIG. 54, different building blocks, cells, microgels, cell spheroid, and BHS are used to generate tissue-like structures within 72 h. For a better comparison, four aggregates (cell spheroid/BHS) or the equivalent quantities of their components (cells/microgels) were compared. The initial configuration entails a mixture of cells and microgels, allowed to form BHS on a planar substrate without any geometric constraints. The design variables were cell density (480,000 cells, equivalent to 4 cell spheroids/BHS), microgel concentration (108 μL, equivalent to 4 BHS), and culture time (72 h), using which formed aggregates had R_{eq} of 139±82 μm. Changing the variables may results in formation of different sizes of aggregates, which have been reported. The second configuration involved fusing cell spheroid on a flat untreated surface, which yielded constructs with R_{eq} of 330±18 μm. While cell spheroids have been used as building blocks for tissue engineering scaffolds and biofabrication, their inherent limitations, such as hypoxic core formation and compactness/density, render the fabrication of viable large tissue-like structures non-trivial, unless they are perfusable or vascularized.

The third configuration involves cell spheroid and microgels, which have been reported for tissue engineering applications. For formation of a tissue-like structure, the connectivity among microgels and cell spheroid is crucial, which is regulated by the ratio of cell spheroid to microgel size/density. In our experiments, using four cell spheroids leads to the microgels accumulation on the surface of the cell spheroid. This microgel coating impeded cell spheroid fusion blocking the formation of constructs at the mm scale. Finally, BHS were allowed to contact, resulting in mm scale (R_{eq}=1339±119 μm) fused tissue-like structures that could be pipetted, indicative of the unique capacity of BHS for large-scale tissue fabrication with sizes reaching up to 4 mm in diameter in this work. All together, radar plots in FIG. 54 compare BHS as building blocks for fabricating large tissue-like constructs in vitro with cell/microgels, cell spheroid, and cell spheroid/microgels based on cell-matrix interactions, scalability, structural integrity, cell viability/metabolic activity, modularity and building block fusion for tissue formation.

It should be understood that the disclosure of a range of values is a disclosure of every numerical value within that range, including the end points. It should also be appreciated that some components, features, and/or configurations may be described in connection with only one particular embodiment, but these same components, features, and/or configurations can be applied or used with many other embodiments and should be considered applicable to the other embodiments, unless stated otherwise or unless such a component, feature, and/or configuration is technically impossible to use with the other embodiment. Thus, the components, features, and/or configurations of the various embodiments can be combined together in any manner and such combinations are expressly contemplated and disclosed by this statement.

It will be apparent to those skilled in the art that numerous modifications and variations of the described examples and embodiments are possible considering the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof.

It should be understood that modifications to the embodiments disclosed herein can be made to meet a particular set of design criteria. Therefore, while certain exemplary embodiments of the apparatus and methods of using and making the same disclosed herein have been discussed and illustrated, it is to be distinctly understood that the invention is not limited thereto but may be otherwise variously embodied and practiced within the scope of the following claims.

What is claimed is:

1. A method of forming porous microgels, the method comprising:
crosslinking first polymers and second polymers to form composite microgels;
adding the composite microgels to a liquid solution at a first temperature to form a composite microgel suspension;
reducing the temperature of the composite microgel suspension to a second temperature below a phase separation temperature such that the second polymers fully or partially separate from the first polymers; and
filtering the composite microgel suspension from the liquid solution such that the second polymers diffuse out of the composite microgel suspension, resulting in the porous microgels.

2. The method of claim 1, wherein the first polymers and the second polymers are two different polymers selected from the group consisting of hyaluronic acid, polyethylene glycol, and gelatin methacryloyl.

3. The method of claim 1, wherein the first polymers are gelatin methacryloyl and the second polymers are polyethylene glycol.

4. A porous microgel formed from the method of claim 1.

5. The porous microgel of claim 4, wherein voids of the porous microgel are between 5 and 40 μm.

6. A method of forming a porous granular hydrogel scaffold, the method comprising:
providing porous microgels according to the method of claim 1; and
crosslinking the porous microgels to form the porous granular hydrogel scaffold.

7. The method of claim 6, wherein crosslinking the porous microgels comprises physical crosslinking.

8. The method of claim 6, wherein crosslinking the porous microgels comprises chemical crosslinking.

9. The method of claim 6, wherein crosslinking the porous microgels comprises non-light-mediated crosslinking.

10. A porous granular hydrogel scaffold formed from the method of claim 6.

11. The porous granular hydrogel scaffold of claim 10, wherein the porous granular hydrogel scaffold has a void fraction between 15% and 60%.

12. A method of forming a porous granular hydrogel scaffold, the method comprising:
providing porous microgels according to the method of claim 1;
combining the porous microgels with adherent cells to form hybrid microgel aggregates; and
crosslinking the hybrid microgel aggregates to form the porous granular hydrogel scaffold.

13. The method of claim 12, wherein crosslinking the porous microgels comprise physical crosslinking.

14. The method of claim 12, wherein crosslinking the porous microgels comprise chemical crosslinking.

15. The method of claim 12, wherein crosslinking the porous microgels comprises non-light-mediated crosslinking.

16. The method of claim 12, wherein the hybrid microgel aggregates have void fractions between 3 and 30%.

17. A method for regenerating tissue, the method comprising:
providing porous microgels according to the method of claim 1;
injecting the porous microgels at an injection site within the tissue; and
crosslinking the porous microgels to form the porous granular hydrogel scaffold.

18. The method of claim 17, wherein crosslinking the porous microgels comprise physical crosslinking and/or chemical crosslinking.

19. The method of claim 17, wherein crosslinking the porous microgels comprises a non-light-mediated crosslinking, and wherein the injection site does not have access to light.

20. The method of claim 17, wherein the tissue is selected from the group consisting of nervous tissue, endothelial tissue, epithelial tissue, muscle tissue, and connective tissue.
